(19) United States
(12) Patent Application Publication
(10) Pub. No.: US 2017/0129956 A1
(43) Pub. Date: May 11, 2017

(54) ANTI-SIGLEC-15 ANTIBODIES FOR USE IN TREATMENT OF OSTEOGENESIS IMPERFECTA

(71) Applicant: Daiichi Sankyo Company, Limited, Tokyo (JP)

(72) Inventors: Mario FILION, Quebec (CA); Gilles Bernard TREMBLAY, Quebec (CA); Anna N. MORAITIS, Quebec (CA)

(73) Assignee: Daiichi Sankyo Company, Limited, Tokyo (JP)

(21) Appl. No.: 15/318,771

(22) PCT Filed: Jun. 17, 2015
(86) PCT No.: PCT/CA2015/000396

§ 371 Date: Dec. 14, 2016

Related U.S. Application Data
(60) Provisional application No. 62/013,582, filed on Jun. 18, 2014.

(51) Int. Cl. C07K 16/28

(52) U.S. Cl. CPC C07K 16/2851; A61K 2039/505

(57) ABSTRACT

The present invention relates to the treatment of osteogenesis imperfecta with anti-Siglec-15 antibodies or antigen binding fragment thereof. Treatment of osteogenesis imperfecta type VI is particularly contemplated.

### FIELD OF THE INVENTION

The present invention relates to the treatment of osteogenesis imperfecta with anti-Siglec-15 antibodies or antigen-binding fragment thereof. Treatment of osteogenesis imperfecta type VI is particularly contemplated.

### BACKGROUND OF THE INVENTION

Bone is a dynamic connective tissue comprised of functionally distinct cell populations required to support the structural, mechanical and biochemical integrity of bone and the human body's mineral homeostasis. The principal cell types involved include, osteoblasts responsible for bone formation and maintaining bone mass, osteoclasts responsible for bone resorption and osteocytes which are thought to be mechanosensor cells that control the activity of osteoblasts and osteoclasts. Osteoblasts and osteoclasts function in a dynamic process termed bone remodeling. The development and proliferation of these cells from their progenitors is governed by networks of growth factors and cytokines produced in the bone microenvironment as well as by systemic hormones. Bone remodeling is ongoing throughout the lifetime of the individual and is necessary for the maintenance of healthy bone tissue and mineral homeostasis. The process remains largely in equilibrium and is governed by a complex interplay of systemic hormones, peptides and downstream signaling pathway proteins, local transcription factors, cytokines, growth factors and matrix remodeling genes.

An interference or imbalance arising in the bone remodeling process can produce skeletal disease, with the most common skeletal disorders characterized by a net decrease in bone mass. A primary cause of this reduction in bone mass is an increase in osteoclast number and/or activity.

Since bone remodelling takes place in discrete packets throughout the skeleton, locally produced hormones and enzymes may be more important than systemic hormones for the initiation of bone resorption and the normal remodelling process. Such local control is mediated by osteoblasts and osteoclasts in the microenvironment in which they operate. For example, osteoclasts attach to the bone matrix and form a separate compartment between themselves and the bone surface delimited by a sealing zone formed by a ring of actin surrounding the ruffled border. Multiple small vesicles transport enzymes toward the bone matrix and internalize partially digested bone matrix. The microenvironment within the sealing zone is rich with the presence of lysosomal enzymes and is highly acidic compared to the normal physiological pH of the body. The ruffled border membrane also expresses RANK, the receptor for RANKL, and macrophage-colony stimulating factor (M-CSF) receptor, both of which are responsible for osteoclast differentiation, as well as the calcitonin receptor capable of rapidly inactivating the osteoclast (Baron, R. 2003).

Thus, it stands to reason that the unique local environments created by these specialized cells is due to the expression of either unique genetic sequences not expressed in other tissues and/or splice variants of polynucleotides and polypeptides expressed in other tissues.

Many diseases linked to bone remodelling are poorly understood, generally untreatable or treatable only to a limited extent. For example, there are very limited options for the treatment of osteogenesis imperfecta (OI).

Osteogenesis imperfecta is characterized by bone fragility exhibiting high susceptibility to fractures and encompasses a group of connective tissue disorders caused by mutations in genes encoding type I collagen. Recessive OI type VI is unique among OI types in that it is characterized by an increased amount of unmineralized osteoid, thereby suggesting a distinct disease mechanism. It was recently shown that a mutation in the SERPINF1 gene, which encodes pigment epithelium-derived factor (PEDF), leads to this distinctive form of OI (Homan et al., 2011). Although OI type VI is quite rare, it is considered moderate in severity and causes bone malformation and fractures during infancy, resulting in severely affected mobility by adolescence. This brittle bones disease is due to a delay in the mineralization of the bone characterized by thickening of the osteoid and elevated serum alkaline phosphatase levels. Current treatment for OI includes growth hormones and bisphosphonates, but patients with OI type VI do not respond as well to bisphosphonates. Novel treatments for OI type VI are therefore needed (Homan et al. 2011).

The Applicant has demonstrated that Sialic-acid-binding immunoglobulin-like lectin-15 (Siglec-15) is required for osteoclast differentiation and has described antibodies targeting Siglec-15 for treatment of bone diseases and for inhibiting bone resorption.

Treatment of osteogenesis imperfecta type VI with an anti-Siglec-15 antibody is particularly disclosed herewith.

### SUMMARY OF THE INVENTION

The invention relates to the use of anti-Siglec-15 antibodies or antigen-binding fragment thereof in the treatment of bone diseases associated with an imbalance or dis-regulation of PEDF or of the PEDF pathway.

More particularly, the invention relates to the treatment of bone diseases associated with an inactivation, loss of function, loss of expression, loss of secretion or mutation of PEDF.

The invention also relates to the treatment of bone diseases associated with an inactivation, loss of function, loss of expression, loss of secretion or mutation of a downstream effector of PEDF (e.g., a downstream effector involved in the PEDF-mediated biological effects on bone cells or tissue, such as, for example, vascular endothelial growth factor (VEGF)).

In addition, the invention relates to the treatment of bone diseases associated with loss of function, loss of expression, loss of secretion or mutation of a regulator that positively affects the expression or secretion of PEDF (e.g., angiostatin, kringle 5 domain).

The invention further relates to the treatment of bone diseases associated with a gain of function, expression, secretion of a regulator that negatively affects the expression or secretion of PEDF.

Individuals that may benefit from the treatment include those having a mutation in the SERPINF1 gene. Such individuals particularly include those in which the SERPINF1 gene mutation results in an inactivation, loss of function or loss of expression of the PEDF protein. Such individuals may be identified by testing for mutations in the SERPINF1 gene or in the PEDF protein and/or by testing the activity of the PEDF protein obtained from such individuals (e.g., in vitro or in vivo testing). For exemplary embodiments of SERPINF1 mutations see Homan et al., 2011. Individuals in need especially encompass children or infants.

The invention especially relates to the treatment of osteogenesis imperfecta type VI with an anti-Siglec-15 antibody or an antigen-binding fragment thereof.

Treatment disclosed herein includes administration of an antibody or antigen-binding fragment thereof that is capable of binding to human Siglec-15 (SEQ ID NO.:2) and/or to a variant or a natural variant thereof.

In accordance with the present invention, suitable anti-Siglec-15 antibody or antigen-binding fragment thereof may include those that may be capable of inhibiting an osteoclast differentiation, formation and/or the activity of osteoclasts in vitro and/or in vivo.

### BRIEF DESCRIPTION OF THE DRAWINGS

FIG. 1 shows femoral (A), tibial (B) and vertebral (C) bone mineral density (BMD) analysis using dual-energy x-ray absorption (DEXA) of male SERPINF1 null mice treated with vehicle (PBS), 3 mg/kg (3) and 10 mg/kg (10) anti-Siglec-15 antibody. *, p<0.05. Treatment with 10 mg/kg anti-Siglec-15 resulted in increased BMD in femur (panel A) and tibia (panel B). In the vertebrae (panel C), the same tendencies were observed but the effects were milder.

FIG. 2 shows microCT analysis of bone volume (% BV/TV) and connective density (Conn.Dn/mm³) in the distal femur of male SERPINF1 null mice treated with vehicle (PBS), 3 mg/kg (3) and 10 mg/kg (10) anti-Siglec-15 antibody. *, p<0.05. Representative microCT images of distal femur (panel A). Bone volume (panel B) was increased in animals that were treated with 10 mg/kg anti-Siglec-15. Connective density (panel C) was augmented with anti-Siglec-15 antibody treatment at both 3 mg/kg and 10 mg/kg.

FIG. 3 shows microCT analysis of trabecular number (TbN 1/mm; panel A), trabecular separation (TbSp. mm; panel B) and trabecular thickness (TbTh mm; panel C) in the distal femur of male SERPINF1 null mice treated with vehicle (PBS), 3 mg/kg (3) and 10 mg/kg (10) anti-Siglec-15 antibody. *, p<0.05. Trabecular number was increased in animals treated with 10 mg/kg of anti-Siglec-15 and trabecular separation was correspondingly decreased in this group compared to vehicle control. Trabecular thickness was unaffected by treatment.

FIG. 4 shows microCT analysis of cortical thickness (CtTh mm; panel A) and cortical bone volume (% BV/TV; panel B) in the distal femur of male SERPINF1 null mice treated with vehicle (PBS), 3 mg/kg (3) and 10 mg/kg (10) anti-Siglec-15 antibody. Cortical thickness and bone volume were unaffected by treatment with anti-Siglec-15.

Further scope, applicability and advantages of the present invention will become apparent from the non-restrictive detailed description given hereinafter. It should be understood, however, that this detailed description, while indicating exemplary embodiments of the invention, is given by way of example only, with reference to the accompanying drawings.

### DETAILED DESCRIPTION OF THE INVENTION

The present invention relates to the treatment of osteogenesis imperfecta with anti-Siglec-15 antibodies or antigen-binding fragment thereof. Treatment of osteogenesis imperfecta type VI is particularly contemplated.

In accordance with the present invention, the anti-Siglec-15 antibody or antigen-binding fragment thereof may be capable of inhibiting an osteoclast differentiation, formation and/or the activity of osteoclasts in vitro and/or in vivo.

Such antibodies or antigen-binding fragments thereof may include for example, those that are capable of binding to human Siglec-15 (SEQ ID NO.:2) or to a variant or natural variant thereof.

Such antibodies or antigen-binding fragments thereof also includes those that may be capable of binding to amino acids 20 to 259 of Siglec-15 (SEQ ID NO.:2) and/or to a corresponding region of a Siglec-15 variant (e.g., a variant having at least 80% sequence identity with SEQ ID NO.:2 including, for example, SEQ ID NO.:4). More particularly the antibodies or antigen-binding fragment of the present invention may bind to amino acids 49 to 165 of Siglec-15 (SEQ ID NO.:2) and/or to a corresponding region of a Siglec-15 variant (e.g., a variant having at least 80% sequence identity with SEQ ID NO.:12 including, for example, SEQ ID NO.:4). The antibodies or antigen-binding fragment of the present invention may also include those which are capable of binding to an epitope unique to human Siglec-15 including, for example, an epitope comprising the arginine located at position 99 (R99) of SEQ ID NO.:2.

It is to be understood herein that antibodies that preferably bind human Siglec-15 (SEQ ID NO.:2) over mouse Siglec-15 (SEQ ID NO.:4) may be more effective at inhibiting differentiation or activity of human osteoclasts than mouse osteoclasts. An antibody that binds an epitope found in human Siglec-15 and not in mouse Siglec-15, may inhibit differentiation or activity of human osteoclasts and not that of mouse osteoclasts. Potency of anti-Siglec-15 antibodies may thus be tested in monkeys or using cells isolated from monkeys. In fact, Siglec-15 protein of cynomolgus and rhesus monkeys is very similar to that of the human Siglec-15 amino acid sequence and anti-Siglec-15 antibodies may already be tested in these animal species. Therefore, potency assays may be adapted depending on the specificity of the antibody (e.g., towards human, monkey and/or mouse Siglec-15).

The potency may be measured in vitro as indicated below. A suitable antibody may be selected on that basis or based on their potency in a suitable animal model (e.g., see Example 15 of U.S. Pat. No. 8,575,316 or Stuible et al, J. Biol. Chem 289 (10), 6498-6512, 2014.

In an exemplary embodiment, the antibodies or fragments may have therapeutic uses in bone loss associated with bone diseases such as conditions where there is an increase in the bone degradative activity of osteoclasts. In certain instances, the antibodies or antigen-binding fragments may interact with cells that express SEQ ID NO:2 and induce an immunological reaction by mediating ADCC. In other instances, the antibodies and fragments may block the interaction of SEQ ID NO:2 with its natural ligands. In yet other instances, the antibodies and fragments may induce internalization of the protein and/or its degradation. In yet further instances, the antibodies and fragments may deliver a drug (e.g., toxin) to a bone cell by targeting SEQ ID NO.:2.

The antibody or antigen-binding fragment of the invention may be administered (e.g., concurrently, sequentially) with another drug useful for the treatment of bone loss, bone resorption (an anti-resorptive) or useful for the treatment of a disease associated with bone loss or bone resorption.

Antibodies and antigen-binding fragment capable of inhibiting bone loss are described herein and/or have been described in international application Nos. PCT/CA2013/000646 published on Jan. 23, 2014 under No. WO2014/012165, PCT/CA2010/001586 published under No. WO2011/041894 on Apr. 14, 2011, and PCT/CA2007/000210 published under No. WO2007/093042 on Feb. 13, 2007 the entire content of which is incorporated herein by reference. Such antibodies are particularly contemplated for use in the present invention. Particularly suitable antibody species include those having CDRs of the 25E9 antibody and especially those having humanized frameworks. Other particular suitable antibody species includes those having CDRs of the 2568 antibody or those having the CDRs of the #32A1 antibody.

Therefore, the use of antibodies having CDRs identical to the #32A1 antibody (e.g., rat, chimeric or humanized) disclosed in U.S. Pat. No. 8,575,316 (corresponding to SEQ ID NO.;44, SEQ ID NO.:45 (or SEQ ID NO.:97), SEQ ID NO.:46, SEQ ID NO.:47, SEQ ID NO.:48 and SEQ ID NO.:49 in U.S. Pat. No. 8,575,316) or having CDRs identical to the antibody having deposit accession number FERM BP-10999 or FERM BP-11000 in U.S. Pat. No. 8,546,540 (including humanized versions of the above) is also particularly contemplated. The sequence of the CDRs of the #32A1 antibody is provided in SEQ ID NOs.: 103 to 108 and a shorter form of the CDRH2 is provided in SEQ ID NO.:109. In accordance with Hiruma et al., (U.S. Pat. No. 8,546,540), the #32A1 antibody binds to both human and mouse Siglec-15 and inhibits differentiation of mouse and human osteoclasts.

The authors also demonstrated that the #32A1 antibody is active and suitable for in vivo testing in mouse (U.S. Pat. No. 8,575,316). Other specific anti-Siglec-15 antibody species have been disclosed in US2010/0209428A1 published on Aug. 19, 2010, US2011/0268733A1 published on Nov. 3, 2011, PCT/JP2013/059653 published on Oct. 13, 2013 under No. WO/2013/147212, PCT/JP2013/059654 published on Oct. 13, 2013 under No. WO/2013/147213, and PCT/EP2011/005219 published on Apr. 12, 2012 under No. WO2012/045481A2 the entire content of which is incorporated herein by reference.

Those antibodies and antigen-binding fragments thereof that are capable of inhibiting osteoclast differentiation, formation and/or activity in vitro and/or in vivo may be suitable for therapeutic treatment. As such, antibodies and antibody variants having desirable activity towards osteoclasts or osteoclast precursors are encompassed by the present invention and are particularly contemplated.

For example, the antibodies or antigen-binding fragment of the present invention may be capable of interfering with (inhibiting) differentiation of an osteoclast precursor cell into a differentiated osteoclast.

In accordance with the present invention, the antibody or antigen-binding fragment may be, for example, a polyclonal antibody, a monoclonal antibody, a chimeric antibody, a humanized antibody, a human antibody, a hybrid antibody or a fragment thereof.

Hybrid antibodies encompassed by the present invention include antibodies having at least one immunoglobulin chain (light chain or heavy chain) comprising a humanized variable domain while the other variable domains may be non-humanized (e.g., mouse variable domain).

The constant region or fragment thereof may be from an IgG1, IgG2, IgG3, or IgG4 and especially from a human IgG1, IgG2, IgG3, or IgG4. In a more specific embodiment, the constant region may be from an IgG2 (e.g., human IgG2). In a particular embodiment the constant region may be from an IgG1 (e.g., human IgG1).

The constant region of the light chain may be a lambda constant region or a kappa constant region.

Antigen-binding fragments which may be particularly be useful include, for example, a FV (scFv), a Fab, a Fab′ or a (Fab′)₂.

The antibodies or antigen-binding fragments may be produced in or from an isolated mammalian cell (other than an hybridoma cell) or in an hybridoma cell. An exemplary embodiment of an isolated mammalian cell is an animal cell (e.g., CHO, NSO, etc.) a human cell (e.g., PER.C6™, etc.) (see for example, Li, F. et al., Mabs 2(5):466-477, 2010).

In an aspect of the invention, the antibody or antigen-binding fragment of the present invention may interfere (inhibit) with the differentiation of a human osteoclast precursor cell into a differentiated human osteoclast.

The term “antibody” refers to intact antibody, monoclonal or polyclonal antibodies. The term “antibody” also encompasses, multispecific antibodies such as bispecific antibodies. Human antibodies are usually made of two light chains and two heavy chains each comprising variable regions and constant regions. The light chain variable region comprises 3 CDRs, identified herein as CDRL1, CDRL2 and CDRL3 flanked by framework regions. The heavy chain variable region comprises 3 CDRs, identified herein as CDRH1, CDRH2 and CDRH3 flanked by framework regions.

The term “antigen-binding fragment”, as used herein, refers to one or more fragments of an antibody that retain the ability to bind to an antigen (e.g., SEQ ID NO:2 or variants thereof). It has been shown that the antigen-binding function of an antibody can be performed by fragments of an intact antibody. Examples of binding fragments encompassed within the term “antigen-binding fragment” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR), e.g., V_{H} CDR3. Furthermore, although the two domains of the Fv fragment, V_{L} and V_{H}, are coded for by separate genes, they can be joined, using recombinant methods, by a synthetic linker that enables them to be made as a single polypeptide chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. Furthermore, the antigen-binding fragments include binding-domain immunoglobulin fusion proteins comprising (i) a binding domain polypeptide (such as a heavy chain variable region, a light chain variable region, or a heavy chain variable region fused to a light chain variable region via a linker peptide) that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The hinge region may be modified by replacing one or more cysteine residues with serine residues so as to prevent dimerization. Such binding domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

A typical antigen-binding site is comprised of the variable regions formed by the pairing of a light chain immunoglobulin and a heavy chain immunoglobulin. The structure of the antibody variable regions is very consistent and exhibits very similar structures. These variable regions are typically comprised of relatively homologous framework regions (FR) interspaced with three hypervariable regions termed Complementarity Determining Regions (CDRs). The overall binding activity of the antigen-binding fragment is often dictated by the sequence of the CDRs. The FRs often play a role in the proper positioning and alignment in three dimensions of the CDRs for optimal antigen-binding.

Antibodies and/or antigen-binding fragments of the present invention may originate, for example, from a mouse, a rat or any other mammal or from other sources and may be produced by different means including through recombinant DNA technologies.

The antibodies or antigen-binding fragments may have therapeutic uses in the treatment of bone loss.

### Exemplary In Vitro Potency Assays

### Cell Culture Used for Potency Assays

To induce osteoclast differentiation, mouse RAW264.7 cells (ATCC, Manassas, Va.), grown in DMEM containing 10% fetal calf serum (Gibco) and 1 mM sodium pyruvate, are scraped and resuspended in PBS. Cells are plated at 2×10⁴ cells/cm² in media containing 100 ng/ml mouse RANKL (R&D Systems, Minneapolis, Minn.). Cells are allowed to differentiate for 3 days (for immunofluorescence microscopy) or 4 days (for all other experiments). Human osteoclast precursors (CD14+ peripheral blood mononuclear cells (PBMCs)) are isolated from normal human PBMCs (AllCells, Emeryville, Calif.) using CD14 microbeads and MS columns (Miltenyi Biotec, Cologne, Germany) following the manufacturer's instructions. Cells are plated at 3.1×10⁵ cells/cm² in Alpha-MEM (Gibco) containing 10% fetal calf serum (HyClone), 1 mM sodium pyruvate (HyClone), 25 ng/ml human MCSF and 30 ng/ml human RANKL (R&D Systems). Cells are allowed to differentiate for 7 days, with half of the media replaced on Day 4.

### Osteoclast TRAP Staining and In Vitro Functional Assays

To test the effect of antibodies on osteoclast differentiation and function, cells are induced to differentiate, as described above, in media containing desired concentrations of antibodies (e.g., 0.01 to 10 μg/ml). Osteoclasts are visualized after four days in culture by TRAP staining: briefly, cells are fixed in 3.7% formaldehyde, permeabilized with 0.2% Triton X-100/PBS, and incubated in TRAP staining buffer (100 mM sodium acetate, pH 5.2, 50 mM sodium tartrate, 0.01% Naphthol ASMX and 0.06% Fast Red Violet) for approximately 30 min at 37 C. The TRAP enzyme generates a red reaction product in osteoclasts. To test osteoclast resorption activity, cells are seeded in wells coated with a calcium phosphate substrate (Osteologic, BD BioSciences or OsteoAssay, Corning) and induced to differentiate as above. After 7 days, wells are treated with bleach to remove cells, and areas of substrate resportion are observed by light microscopy. Antibodies that are able to block the activity of Siglec-15 (in osteoclast or in osteoclast precursor cells) may show, for example, fewer TRAP-positive multinucleated cells or may result in an altered morphology of the TRAP-positive multinucleated cells in comparison with a control antibody. As osteoclasts actively digest mineralized substrate, antibodies that are able to block the activity of Siglec-15 (in osteoclast or in osteoclast precursor cells) may show, for example, fewer areas where the calcium substrate has been digested (denuded area) in comparison with a control (e.g., antibodies that do not bind to Siglec-15, absence of antibodies etc.). When the human osteoclasts are differentiated on a calcium phosphate substrate, which acts as a bone-like surface, cells treated with the control antibody usually generate large areas of denuded calcium phosphate indicating that the osteoclasts exhibited resorptive activity. By contrast, cells treated with suitable anti-Siglec-15 antibodies do not resorb the substrate and may sometimes be comparable to the undifferentiated precursor cells.

Another technique involves CD14+ PBMCs that are differentiated into osteoclasts and plated on bovine cortical bone slices (differentiation may be done before plating, upon plating or after plating). The anti-Siglec-15 is added and resorption pits generated on the bone slice surface are observed by reflected light microscopy. Antibodies that are able to block the activity of Siglec-15 (in osteoclast or in osteoclast precursor cells) may result, for example, in fewer or smaller resorption pits.

### Antibodies and Antibody Variants

Anti-Siglec-15 antibodies or antigen-binding fragment thereof that may be suitable for the treatment of bone diseases associated with an imbalance or dis-regulation of PEDF or of the PEDF pathway may include those that are capable of inhibiting osteoclast differentiation, formation and/or activity. Particular embodiments of inhibitory antibodies include, for example, those disclosed herein. Other particular embodiments of inhibitory antibodies include those disclosed in international application Nos. PCT/CA2013/000646, PCT/CA2010/001586, PCT/CA2007/000210, PCT/JP2013/059653, PCT/JP2013/059654, PCT/EP2011/005219, US2010/0209428A1 or US2011/0268733 or antibody variants derived from such antibodies.

Particularly suitable antibody species include those having the CDRs of the 25E9 antibody and especially those having humanized framework regions. Other suitable antibody species may include those having the CDRs of the #32A1 antibody and especially those having humanized framework regions.

In accordance with the present invention, suitable antibodies or antigen-binding fragment thereof may particularly be able to inhibit osteoclast differentiation.

Further in accordance with the present invention, suitable antibodies or antigen-binding fragment thereof may be able to inhibit osteoclast formation.

Also in accordance with the present invention, suitable antibodies or antigen-binding fragment thereof may be able to inhibit osteoclasts activity.

Assays for characterizing the inhibitory activity of a compound (e.g., an anti-Siglec-15 antibody or an antigen-binding fragment thereof) have been described in WO2007/093042 and/or are known to a person of skill in the art (e.g., Buckley K. A. et al. 2005; Collin-Osdoby P. et al, 2003). Such assay includes for example, testing the effect of an anti-Siglec-15 antibody on the osteoclast differentiation process (e.g., in the presence of RANKL). The assay may include determining the number of differentiated osteoclasts, their level of multi-nucleation, testing for specific markers of differentiated osteoclasts (e.g., TRAP) and/or testing the resorbing activity of osteoclasts on bone matrix in the presence or absence of the compound. Osteoclast precursors cells suitable for carrying out such testing are available and include, for example, RAW 264.7 cells or human osteoclasts isolated from peripheral blood monocytes (e.g., Buckley K. A. et al. 2005; Collin-Osdoby P. et al, 2003).

Further in accordance with the present invention, the antibodies or antigen-binding fragment thereof may be able to inhibit bone resorption (e.g., bone resorption activity of osteoclasts).

Accordingly, the present invention provides in one aspect, an antibody or antigen-binding fragment thereof capable of specific binding to Siglec-15 which may have a light chain variable region at least 80% identical to SEQ ID NO.:6 and/or a heavy chain variable region at least 80% identical to SEQ ID NO.:12. The antibody or antigen-binding fragment thereof may also comprise at least one amino acid substitution in comparison with SEQ ID NO.:6 or SEQ ID NO.:12.

The present invention also provides in another aspect, an antibody or antigen-binding fragment thereof which may have a light chain variable region at least 80% identical to SEQ ID NO.:22 and/or a heavy chain variable region at least 80% identical to SEQ ID NO.:26. The antibody or antigen-binding fragment thereof may also comprise at least one amino acid substitution in comparison with SEQ ID NO.:22 or SEQ ID NO.:26.

In accordance with the present invention, the amino acid substitution may be an amino acid appearing at a corresponding position in a natural human antibody.

In accordance with an embodiment of the invention, the amino acid substitution may be outside of a complementarity determining region (CDR).

In accordance with an embodiment of the invention, the antibody the amino acid substitution may be located, for example, in the light chain variable region.

In accordance with an additional embodiment of the invention, the antibody or antigen-binding fragment thereof may comprise at least two or at least three amino acid substitutions. Such amino acid substitutions may be located in the same variable region or may be located in distinct variable regions.

Further in accordance with the present invention, the antibody or antigen-binding fragment thereof may comprise for example, from one to twenty-five amino acid substitutions in the light chain variable region and/or heavy chain variable region. More particularly, the antibody or antigen-binding fragment thereof may have, for example, from one to twenty-two amino acid substitution in its light chain variable region and from one to twenty-five amino acid substitutions in its heavy chain variable region.

Antibodies or antigen-binding fragments comprising the complementarity determining regions of SEQ ID NO.:6 and the complementarity determining regions of SEQ ID NO.:12 are particularly contemplated. Complementarity determining regions of SEQ ID NO.:6 and SEQ ID NO.:12 may be as set forth in SEQ ID NOs.: 47 to 52.

Other exemplary embodiments of antibodies or antigen-binding fragments include those having the complementarity determining regions of SEQ ID NO.:95 and the complementarity determining regions of SEQ ID NO.:96. Complementarity determining regions of SEQ ID NO.: 95 and SEQ ID NO.:96 may be as set forth in SEQ ID NOs.:97 to 102.

It is to be particularly noted that the only difference between the mouse 25E9 antibody and the mouse 2568 antibody variable domains reside in CDRH3. Both antibodies are functional at inhibiting osteoclast differentiation in vitro. Therefore, it seems that the 25E9 CDRH3 may tolerate some level of amino acid substitution (1, 2, 3 or 4 substitutions without losing its in vitro activity. The substitutions may be, for example, conservative amino acid substitutions.

Antibodies or antigen-binding fragments comprising the complementarity determining regions of SEQ ID NO.:22 and the complementarity determining regions of SEQ ID NO.:26 are also contemplated. Complementarity determining regions of SEQ ID NO.:22 and SEQ ID NO.:26 may be as set forth in SEQ ID Nos. 53 to 58.

Antibodies or antigen-binding fragments comprising the complementarity determining regions of the #32A1 antibody are also contemplated (e.g., SEQ ID NOs.: 103-109).

Such antibodies or antigen-binding fragments may comprise framework amino acids of a human antibody and thus particularly include humanized form of the above-antibodies.

Therefore, antibodies that may be suitable for the present invention includes those having:
a) a light chain comprising the complementarity determining region amino acid sequences set forth in SEQ ID NO.:47, SEQ ID NO.:48 and SEQ ID NO.:49 and a heavy chain comprising the complementarity determining region amino acid sequences set forth in SEQ ID NO.:50, SEQ ID NO.:51 and SEQ ID NO.:52;
b) a light chain comprising the complementarity determining region amino acid sequences set forth in SEQ ID NO.:53, SEQ ID NO.:54 and SEQ ID NO.:55 and a heavy chain comprising the complementarity determining region amino acid sequences set forth in SEQ ID NO.: 56, SEQ ID NO.:57 and SEQ ID NO.:58;
c) a light chain comprising the complementarity determining region amino acid sequences set forth in SEQ ID NO.:97, SEQ ID NO.:98 and SEQ ID NO.:99 and a heavy chain comprising the complementarity determining region amino acid sequences set forth in SEQ ID NO.:100, SEQ ID NO.:101 and SEQ ID NO.:102, or;
d) a light chain comprising the complementarity determining region amino acid sequences set forth in SEQ ID NO.:103, SEQ ID NO.: 104 (or SEQ ID NO.:109) and SEQ ID NO.:105 and a heavy chain comprising the complementarity determining region amino acid sequences set forth in SEQ No.:106, SEQ ID NO.:107 and SEQ ID NO.:108.

Other exemplary embodiments of the invention includes for example an antibody or an antigen-binding fragment thereof having a light chain variable domain as set forth in SEQ ID NO.:33 (Generic 25E9 light chain variable domain (consensus 1)).

(SEQ ID NO.: 33)
DIVMTQXXXSXPVTPGEXXSISCRSTKSLLHSNGNTYLYWXLQXPGQSP
QLLIYRMSNLASGVPDRFSGSGSGTXFTLXISRVEAEDVGVYYCMQHLE
YPFTFGGGTKXEIK;

wherein at least one of the amino acid identified by X may be an amino acid substitution in comparison with a corresponding amino acid in the polypeptide set forth in SEQ ID NO.:6 (the mouse VL). The amino acid substitution may be, for example conservative or non-conservative. In accordance with the invention, the amino acid substitution may be conservative.

Another exemplary embodiment of the invention includes for example an antibody or an antigen-binding fragment thereof having a light chain variable domain as set forth in SEQ ID NO.:34 (Generic 25E9 light chain variable domain (consensus 2)).

(SEQ ID NO.: 34)
DIVMTQXₐ₁Xₐ₂Xₐ₃SXₐ₄PVTPGEXₐ₅Xₐ₆SISCRSTKSLLHSNGNTYLYW
Xₐ₇LQXₐ₈PGQSPQLLIYRMSNLASGVPDRFSGSGSGTXₐ₉FTLXₐ₁₀ISR
VEAEDVGVYYCMQHLEYPFTFGGGTKXₐ₁₁EIK;

wherein at least one of the amino acid identified by X may be an amino acid substitution in comparison with a corresponding amino acid in the polypeptide set forth in SEQ ID NO.:6 (the mouse VL) and;

wherein Xa1, Xa4, Xa7, Xa8, Xa10 and Xa11 may each independently be a conservative amino acid substitution in comparison with SEQIDNO.6;

wherein Xa2, Xa5, Xa6 may each independently be a semi-conservative amino acid substitution in comparison with SEQIDNO.6;

wherein Xa3 may be P or L; and

wherein Xa9 may be A or D.

Yet another exemplary embodiment of the invention includes for example, an antibody or an antigen-binding fragment thereof having a light chain variable domain as set forth in SEQ ID NO.:35 (Generic 25E9 light chain variable domain (consensus 3)).

(SEQ ID NO.: 35)
DIVMTQXₐ₁Xₐ₂Xₐ₃SXₐ₄PVTPGEXₐ₅Xₐ₆SISCRSTKSLLHSNGNTYLYW
Xₐ₇LQXₐ₈PGQSPQLLIYRMSNLASGVPDRFSGSGSGTXₐ₉FTLXₐ₁₀ISR
VEAEDVGVYYCMQHLEYPFTFGGGTKXₐ₁₁EIK;

wherein at least one of the amino acid identified by X (including Xa1 to Xa11) may be an amino acid substitution in comparison with a corresponding amino acid in the polypeptide set forth in SEQ ID NO.:6 (the mouse VL) and

wherein Xa1 may be A or S;

wherein Xa2 may be A or P;

wherein Xa3 may be P or L;

wherein Xa4 may be a hydrophobic amino acid (e.g., V or L);

wherein Xa5 may be S or P;

wherein Xa6 may be a hydrophobic amino acid (e.g., V or A);

wherein Xa7 may be an aromatic amino acid (e.g. F or Y);

wherein Xa8 may be a basic amino acid (e.g., R or K);

wherein Xa9 may be A or D;

wherein Xa10 may be a basic amino acid (e.g., R or K); and

wherein Xa11 may be a hydrophobic amino acid (e.g., L or V).

In a further embodiment, the present invention includes for example, an antibody or an antigen-binding fragment thereof, having a heavy chain variable domain as set forth in SEQ ID NO.:36 (Generic 25E9 heavy chain variable domain (consensus 1)).

(SEQ ID NO.: 36)
EIQLQQSGXEXXXPGXSVXXSCKASGYTFTDYDMHWVXQXPXXGLEWXGT
IDPETGGTAYNQKFKGXXTXTADXSXXTAYMELSSLXSEDXAVYYCTSFY
YTYSNYDVGFAYWGQGTLVTVSX;

wherein at least one of the amino acid identified by X may be an amino acid substitution in comparison with a corresponding amino acid in the polypeptide set forth in SEQ ID NO.:12 (the mouse VH). The amino acid substitution may be, for example conservative or non-conservative. In accordance with the invention, the amino acid substitution may be conservative.

Yet a further embodiment of the present invention includes for example, an antibody or an antigen-binding fragment thereof having a heavy chain variable domain as set forth in SEQ ID NO.:37 (Generic 25E9 heavy chain variable domain (consensus 2)).

(SEQ ID NO.: 37)
EIQLQQSGX_{b1}EX_{b2}X_{b3}X_{b4}PGX_{b5}SVX_{b6}X_{b7}SCKASGYTFTDYDMHWVX_{b8}
QX_{b9}P_{Xb10}X_{b11}GLEWX_{b22}GTIDPETGGTAYNQKFKGX_{b13}X_{b14}TX_{b15}TAD
X_{b16}SX_{b17}X_{b18}TAYMELSSLX_{b19}SEDX_{b20}AVYYCTSFYYTYSNYDVGFA
YWGQGTLVIVSX_{b21};

wherein at least one of the amino acid identified by X (including Xb1 to Xb21) may be an amino acid substitution in comparison with a corresponding amino acid in the polypeptide set forth in SEQ ID NO.:12 (the mouse VH) and

wherein Xb2, Xb4, Xb5, Xb7, Xb8, Xb9, Xb11, Xb12, Xb13, Xb15, Xb16, Xb17, Xb18, Xb20 and Xb21 may each independently be a conservative amino acid substitution in comparison with SEQIDNO.12;

wherein Xb1, Xb6, Xb14 may each independently be a semi-conserved amino acid substitution in comparison with SEQIDNO.:12 (the mouse VH);

wherein Xb3 may be V or K;

wherein Xb10 may be V or G; and

wherein Xb19 may be T or R.

Another embodiment of the invention includes, for example, an antibody or an antigen-binding fragment having an heavy chain variable domain as set forth in SEQ ID NO.:38 (Generic 25E9 heavy chain variable domain (consensus 3)).

(SEQ ID NO.: 38)
EIQLQQSGX_{b1}EX_{b2}X_{b3}X_{b4}PGX_{b5}SVX_{b6}X_{b7}SCKASGYTFTDYDMHWV
X_{b8}QX_{b9}P_{Xb10}X_{b11}GLEWX_{b12}GTIDPETGGTAYNQKFKGX_{b13}X_{b14}TX_{b15}
TADX_{b16}SX_{b17}X_{b18}TAYMELSSLX_{b19}SEDX_{b20}AVYYCTSFYYTYSNYD
VGFAYWGQGTLVTVSX_{b21};

wherein at least one of the amino acid identified by X (including Xb1 to Xb21) may be an amino acid substitution in comparison with a corresponding amino acid in the polypeptide set forth in SEQ ID NO.:12 (the mouse VH) and;

wherein Xb1 may be a hydrophobic amino acid (e.g., V or A);

wherein Xb2 may be a hydrophobic amino acid (e.g., L or V);

wherein Xb3 may be V or K;

wherein Xb4 may be a basic amino acid (e.g., R or K);

wherein Xb5 may be A or S;

wherein Xb6 may be T or K;

wherein Xb7 may be a hydrophobic amino acid (e.g., L or V);

wherein Xb8 may be a basic amino acid (e.g., K or R);

wherein Xb9 may be T or A;

wherein Xb10 may be V or G;

wherein Xb11 may be a basic amino acid (e.g., H or Q);

wherein Xb12 may be a hydrophobic amino acid (e.g., I or M);

wherein Xb13 may be a basic amino acid (e.g., K or R);

wherein Xb14 may be a hydrophobic amino acid (e.g., A or V);

wherein Xb15 may be a hydrophobic amino acid (e.g., L or I);

wherein Xb16 may be a basic amino acid (e.g., R or K);

wherein Xb17 may be a neutral hydrophilic amino acid (e.g., S or T);

wherein Xb18 may be a neutral hydrophilic amino acid (e.g., T or S);

wherein Xb19 may be T or R;

wherein Xb20 may be a neutral hydrophilic amino acid (e.g., S or T); and

wherein Xb21 may be A or S.

Other exemplary embodiments of the invention include, for example, an antibody or an antigen-binding fragment thereof, having a light chain variable domain set forth in SEQ ID NO.: 39 (Generic 25D8 light chain variable domain (consensus 1)).

(SEQ ID NO.: 39)
DIVMTQXXXSXPVTXGXXASISCRSSKSLLHSNGITYLYWYLQKPGQSPQ
LLIYQMSNLASGVPDRFSXSGSGTDFTLXISRVEAEDVGVYYCAQNLELP
YTFGGGTKXEIK;

wherein at least one of the amino acid identified by X may be an amino acid substitution in comparison with a corresponding amino acid in the polypeptide set forth in SEQ ID NO.:22 (the mouse VL). The amino acid substitution may be, for example conservative or non-conservative. In accordance with the invention, the amino acid substitution may be conservative.

Yet another exemplary embodiment of the invention includes, for example, an antibody or antigen-binding fragment thereof, having a light chain variable domain set forth in SEQ ID NO.: 40 (Generic 25D8 light chain variable domain (consensus 2)).

(SEQ ID NO.: 40)
DIVMTQX_{c1}X_{c2}X_{c3}SX_{c4}PVTX_{c5}GX_{c6}X_{c7}ASISCRSSKSLLHSNGITYLYW
YLQKPGQSPQLLIYQMSNLASGVPDRFSX_{c8}SGSGTDFTLX_{c9}ISRVEAE
DVGVYYCAQNLELPYTFGGGTKX_{c10}EIK;

wherein at least one of the amino acid identified by X may be an amino acid substitution in comparison with a corresponding amino acid in the polypeptide set forth in SEQ ID NO.:22 (the mouse VL) and

wherein Xc1, Xc3, Xc9 and Xc10 may each independently be a conservative amino acid substitution in comparison with SEQ ID NO.:22;

wherein Xc2, Xc7, Xc8 may each independently be a semi-conservative amino acid substitution in comparison with SEQ ID NO.: 22;

Wherein Xc4 may be N or L;

Wherein Xc5 may be L or P; and

Wherein Xc6 may be T or E.

An additional embodiment of the present invention includes for example, an antibody or antigen-binding fragment thereof, having a light chain variable domain set forth in SEQ ID NO.: 41 (Generic 25D8 light chain variable domain (consensus 3)).

(SEQ ID NO.: 41)
DIVMTQX_{c1}X_{c2}X_{c3}SX_{c4}PVTX_{c5}GX_{c6}X_{c7}ASISCRSSKSLLHSNGITYLY
WYLQKPGQSPQLLIYQMSNLASGVPDRFSX_{c8}SGSGTDFTLX_{c9}ISRVE
AEDVGVYYCAQNLELPYTFGGGTKX_{c10}EIK;

wherein at least one of the amino acid identified by X may be an amino acid substitution in comparison with a corresponding amino acid in the polypeptide set forth in SEQ ID NO.:22 (the mouse VL) and

Wherein Xc1 may be A or T;

Wherein Xc2 may be A or P;

Wherein Xc3 may be F or L;

Wherein Xc4 may be N or L;

Wherein Xc5 may be L or P;

Wherein Xc6 may be T or E;

Wherein Xc7 may be S or P;

Wherein Xc8 may be S or G;

Wherein Xc9 may be a basic amino acid (e.g., R or K); and

Wherein Xc10 may be a hydrophobic amino acid (e.g., L or V).

Yet an additional embodiment of the present invention includes for example, an antibody or antigen-binding fragment thereof, having a heavy chain variable domain set forth in SEQ ID NO.: 42 (Generic 25D8 heavy chain variable domain (consensus 1)).

(SEQ ID NO.: 42)
QVQXQQXGAEXXKPGXSVKXSCKASGYTFTSYWMHWVXQXPGQGLEWXG
LINPSNARTNYNEKFNTXXTXTXDKSXSTAYMXLSSLXSEDXAVYYCAR
GGDGDYFDYWGQGTTXTVSS;

wherein at least one of the amino acid identified by X may be an amino acid substitution in comparison with a corresponding amino acid in the polypeptide set forth in SEQ ID NO.:26 (the mouse VH). The amino acid substitution may be, for example conservative or non-conservative. In accordance with the invention, the amino acid substitution may be conservative.

In a further embodiment, the present invention includes for example, an antibody or antigen-binding fragment thereof, having a heavy chain variable domain set forth in SEQ ID NO.: 43 (Generic 25D8 heavy chain variable domain (consensus 2)).

(SEQ ID NO.: 43)
QVQX_{d1}QQX_{d2}GAEX_{d3}X_{d4}KPGX_{d5}SVKX_{d6}SCKASGYTFTSYWMHWVX_{d7}Q
X_{d8}PGQGLEWX_{d9}GLINPSNARTNYNEKFNTX_{d10}X_{d11}TX_{d12}TX_{d13}DKS
X_{d14}STAYMX_{d15}LSSLX_{d16}SEDX_{d17}AVYYCARGGDGDYFDYWGQGTT
X_{d18}TVSS;

wherein at least one of the amino acid identified by X may be an amino acid substitution in comparison with a corresponding amino acid in the polypeptide set forth in SEQ ID NO.:26 (the mouse VH) and;

wherein Xd1, Xd3, Xd5, Xd6, Xd7, Xd9, Xd10, Xd12, Xd14, Xd15, Xd17, Xd18 may each independently be a conservative amino acid substitution in comparison with SEQ ID NO.:26;

wherein Xd2, Xd11, Xd13, may each independently be a semi-conservative amino acid substitution in comparison with SEQ ID NO.:26;

wherein Xd4 may be V or K;

wherein Xd8 may be R or A; and;

wherein Xd16 may be T or R.

In yet a further embodiment, the present invention includes, for example, an antibody or antigen-binding fragment thereof, having a heavy chain variable domain set forth in SEQ ID NO.: 44 (Generic 25D8 heavy chain variable domain (consensus 3)).

(SEQ ID NO.: 44)
QVQX_{d1}QOX_{d2}GAEX_{d3}X_{d4}KPGX_{d5}SVKX_{d6}SCKASGYTFTSYWMHWVX_{d7}Q
X_{d8}PGQGLEWX_{d9}GLINPSNARTNYNEKFNTX_{d10}X_{d11}TX_{d12}TX_{d13}DKS
X_{d14}STAYMX_{d15}LSSLX_{d16}SEDX_{d17}AVYYCARGGDGDYFDYWGQGTT
X_{d18}TVSS;

wherein at least one of the amino acid identified by X may be an amino acid substitution in comparison with a corresponding amino acid in the polypeptide set forth in SEQ ID NO.:26 (the mouse VH) and;

wherein Xd1 may be a hydrophobic amino acid (e.g., V or L);

wherein Xd2 may be P or S;

wherein Xd3 may be a hydrophobic amino acid (e.g., L or V);

wherein Xd4 may be V or K;

wherein Xd5 may be A or S;

wherein Xd6 may be a hydrophobic amino acid (e.g., L or V);

wherein Xd7 may be a basic amino acid (e.g., K or R);

wherein Xd8 may be R or A;

wherein Xd9 may be a hydrophobic amino acid (e.g., I or M);

wherein Xd10 may be a basic amino acid (e.g., K or R);

wherein Xd11 may be a hydrophobic amino acid (e.g., A or V);

wherein Xd12 may be a hydrophobic amino acid (e.g., L or I);

wherein Xd13 may be a hydrophobic amino acid (V or A);

wherein Xd14 may be a neutral hydrophilic amino acid (e.g., S or T);

wherein Xd15 may be Q or E;

wherein Xd16 may be T or R.

wherein Xd17 may be a neutral hydrophilic amino acid (e.g., S or T); and

wherein Xd18 may be a hydrophobic amino acid (L or V).

The term “humanized antibody” encompasses fully humanized antibody (i.e., frameworks are 100% humanized) and partially humanized antibody (e.g., at least one variable domain contains one or more amino acids from a human antibody, while other amino acids are amino acids of a non-human parent antibody). Typically a “humanized antibody” contains CDRs of a non-human parent antibody (e.g., mouse, rat, rabbit, non-human primate, etc.) and frameworks that are identical to those of a natural human antibody or of a human antibody consensus. In such instance, those “humanized antibodies” are characterized as fully humanized. A “humanized antibody” may also contain one or more amino acid substitutions that have no correspondence to those of the human antibody or human antibody consensus. Such substitutions include, for example, back mutations (e.g., re-introduction of non-human amino acids) that may preserve the antibody characteristics (e.g., affinity, specificity etc.). Such substitutions are usually in the framework region. A “humanized antibody” optionally also comprise at least a portion of a constant region (Fc) which is typically that of a human antibody. Typically, the constant region of a “humanized antibody” is identical to that of a human antibody.

Of course, any antibody, antigen-binding fragment thereof or antibody portion (light chain or heavy chain variable regions), having an amino acid sequence identical to that described herein is encompassed by the present invention, irrelevant of whether it is obtained via humanization technology, hybridoma technology, transgenic mice technologies (e.g., having human immunoglobulin repertoires), or else.

It is to be understood herein that the framework amino acids of the antibodies of the present invention may be from 80% to 100% (e.g., 85 to 100%; 90 to 100%, 95 to 100%) identical to those of a natural human antibodies. Usually, when a framework amino acid is not identical to a corresponding amino acid of a natural antibody, such amino acid may remain identical to the original amino acid (e.g., a mouse amino acid).

As used herein the term “from one to twenty-five (1 to 25)” includes every individual values and ranges such as for example, 1, 2, 3, and up to 25; 1 to 25; 1 to 24, 1 to 23, 1 to 22, 1 to 21, 1 to 20, 1 to 19; 1 to 18; 1 to 17; 1 to 16; 1 to 15 and so on; 2 to 25, 2 to 24, 2 to 23, 2 to 22, 2 to 21, 2 to 20; 2 to 19; 2 to 18; 2 to 17 and so on; 3 to 25, 3 to 24, 3 to 23, 3 to 22, 3 to 21, 3 to 20; 3 to 19; 3 to 18 and so on; 4 to 25, 4 to 24, 4 to 23, 4 to 22, 4 to 21, 4 to 20; 4 to 19; 4 to 18; 4 to 17; 4 to 16 and so on; 5 to 25, 5 to 24, 5 to 23, 5 to 22, 5 to 21, 5 to 20; 5 to 19; 5 to 18; 5 to 17 and so on, etc.

Likewise, other ranges such as for example, “from one to twenty-two (1 to 22)” includes every individual values and ranges such as for example, 1, 2, 3, and up to 22; 1 to 22, 1 to 21, 1 to 20, 1 to 19, 1 to 18, 1 to 17, 1 to 16, 1 to 15; 1 to 14; 1 to 13; 1 to 12; 1 to 11; 1 to 10 and so on; 2 to 22, 2 to 21, 2 to 20, 2 to 19, 2 to 18, 2 to 17, 2 to 16, 2 to 15; 2 to 14; 2 to 13; 2 to 12 and so on; 3 to 22, 3 to 21, 3 to 20, 3 to 19, 3 to 18, 3 to 17, 3 to 16, 3 to 15; 3 to 14; 3 to 13 and so on; 4 to 22, 4 to 21, 4 to 20, 4 to 19, 4 to 18, 4 to 17, 4 to 16, 4 to 15; 4 to 14; 4 to 13; 4 to 12; 4 to 11 and so on; 5 to 22, 5 to 21, 5 to 20, 5 to 19, 5 to 18, 5 to 17, 5 to 16, 5 to 15; 5 to 14; 5 to 13; 5 to 12 and so on, etc.

In a more specific embodiment of the invention, the number of amino acid substitutions that may be made in a light chain variable region derived from SEQ ID NO.:6 may be for example, from 1 to 11 amino acid substitutions.

In yet a more specific embodiment of the invention, the number of amino acid substitutions that may be made in a heavy chain variable region derived from SEQ ID NO.:12 may be for example, from 1 to 21 amino acid substitutions. In some instances, when considering SEQ ID NO.:12, it may be useful to have at least three amino acid substitutions.

In a further more specific embodiment of the invention, the number of amino acid substitutions that may be made in a light chain variable region derived from SEQ ID NO.:22 may be for example, from 1 to 10 amino acid substitutions.

In yet a further more specific embodiment of the invention, the number of amino acid substitutions that may be made in a heavy chain variable region of SEQ ID NO.:26 may be for example, from 1 to 18 amino acid substitutions.

In accordance with an embodiment of the invention, the acid substitutions may be for example, in the light chain variable region.

In accordance with an embodiment of the invention, the amino acid substitutions may be for example, in the heavy chain variable region.

It is particularly understood that the sequence of the signal peptides disclosed in the present application are provided by means of example only. Such signal peptide may have a different amino acid or nucleic acid sequence then those illustrated in the sequence listing. Suitable signal peptides, including those directing proteins to the secretory pathway, are known to those of skill in the art.

An antibody or antigen-binding fragment may therefore have a light chain variable region having up to twenty-two amino acid substitutions in comparison with SEQ ID NO.:6 or SEQ ID NO.:22 and may have a heavy chain variable region having up to twenty-five amino acid substitutions in comparison with SEQ ID NO.:12 or SEQ ID NO.:26. It is to be understood herein that when the antibody or antigen-binding fragment has two light chain variable regions and two heavy chain variable regions, each one of the light chain variable regions may independently have up to twenty amino acid substitutions and each one of the heavy chain variable regions may have up to twenty amino acid substitutions.

As discussed herein the amino acid substitutions may be conservative or non-conservative. In an exemplary embodiment the amino acid substitutions may be conservative.

It is to be understood herein that the antibody or antigen-binding fragment of the invention may if desired have a portion of the light chain variable region and/or heavy chain variable region set forth in SEQ ID NO.:6, SEQ ID NO.:12, SEQ ID NO.:22 and/or SEQ ID NO.:26. Such antibody or antigen-binding fragment may thus have a portion at an amino- or carboxy-terminus of the light chain variable region and/or heavy chain variable region that originates from a distinct entity (e.g., from a distinct antibody).

Another exemplary embodiment of the antibody or antigen-binding fragment of the present invention includes for example, an antibody or antigen-binding fragment having a light chain variable region which may comprise at least 90 consecutive amino acids of any of SEQ ID NO.:33, SEQ ID NO.:34, SEQ ID NO.:35, SEQ ID NO.:8 or SEQ ID NO.:10.

As used herein the term “at least 90 consecutive amino acids of SEQ ID NO.:33” also includes the terms “at least 91, 92, 93, 94, 95, 96, 97, 98, 99, 100, 101, 102, 103, 104, 105, 106, 107, 108, 109, 110, 111, or at least 112 consecutive amino acids”. The term “at least 90 consecutive amino acids of SEQ ID NO.:33” encompasses any possible sequence of at least 90 consecutive amino acids found in SEQ ID NO.:33 and especially those sequences which include the 3 CDRs of SEQ ID NO.:33, such as, for example a sequence comprising amino acids 6 to 108, 5 to 109, 13 to 103, 14 to 111 of SEQ ID NO.:33 and so on.

As used herein the term “at least 90 consecutive amino acids of SEQ ID NO.:34” also includes the terms “at least 91, 92, 93, 94, 95, 96, 97, 98, 99, 100, 101, 102, 103, 104, 105, 106, 107, 108, 109, 110, 111, or at least 112 consecutive amino acids”. The term “at least 90 consecutive amino acids of SEQ ID NO.:34” encompasses any possible sequence of at least 90 consecutive amino acids found in SEQ ID NO.:34 and especially those sequences which include the 3 CDRs of SEQ ID NO.:34, such as, for example a sequence comprising amino acids 7 to 109, 12 to 104, 22 to 112, 18 to 112 of SEQ ID NO.:34 and so on.

The terms “at least 90 consecutive amino acids of SEQ ID NO.:35”, “at least 90 consecutive amino acids of SEQ ID NO.:8” or “at least 90 consecutive amino acids of SEQ ID NO.:10” have similar meanings.

In accordance with the present invention, the antibody or antigen-binding fragment of the present invention may have, for example, a light chain variable region as set forth in SEQ ID NO.:8 or in SEQ ID NO.:10.

The antibody or antigen-binding fragment of the invention includes (or further includes) for example, a heavy chain variable region which may comprise at least 90 consecutive amino acids of any of SEQ ID NOs.:36, 37, 38, 14, 16, 18 or 20.

As used herein the term “at least 90 consecutive amino acids of SEQ ID NO.:36” also includes the terms “at least 91, 92, 93, 94, 95, 96, 97, 98, 99, 100, 101, 102, 103, 104, 105, 106, 107, 108, 109, 110, 111, or at least 123 consecutive amino acids”. The term “at least 90 consecutive amino acids of SEQ ID NO.:36” encompasses any possible sequence of at least 90 consecutive amino acids found in SEQ ID NO.:36 and especially those sequences which include the 3 CDRs of SEQ ID NO.:36, such as, for example a sequence comprising amino acids 1 to 106, 2 to 112, 11 to 113, 7 to 102 of SEQ ID NO.:36 and so on.

As used herein the term “at least 90 consecutive amino acids of SEQ ID NO.:37” also includes the terms “at least 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122 or at least 123 consecutive amino acids”. The term “at least 90 consecutive amino acids of SEQ ID NO.:37” encompasses any possible sequence of at least 90 consecutive amino acids found in SEQ ID NO.:37 and especially those sequences which include the 3 CDRs of SEQ ID NO.:37, for example a sequence comprising amino acids 6 to 109, 8 to 113, 1 to 102, 2 to 105 of SEQ ID NO.:37 and so on.

The terms “at least 90 consecutive amino acids of SEQ ID NO.:38”, “at least 90 consecutive amino acids of SEQ ID NO.:14, “at least 90 consecutive amino acids of SEQ ID NO.:16”, “at least 90 consecutive amino acids of SEQ ID NO.:18” or “at least 90 consecutive amino acids of SEQ ID NO.:20” have similar meanings.

In accordance with the present invention, the antibody or antigen-binding fragment of the present invention may have, for example, a heavy chain variable region as set forth in SEQ ID NO.:14, 16, 18 or 20.

In accordance with the present invention the antibody or antigen-binding fragment may comprise, for example,
a) a light chain variable region which may comprise at least 90 consecutive amino acids of SEQ ID NO.:33 and a heavy chain variable region which may comprise at least 90 consecutive amino acids of any of SEQ ID NO.:36, SEQ ID NO.:37, SEQ ID NO.:38, SEQ ID NO.:14, SEQ ID NO.:16, SEQ ID NO.:18 or SEQ ID NO.:20;
b) a light chain variable region which may comprise at least 90 consecutive amino acids of SEQ ID NO.:34 and a heavy chain variable region which may comprise at least 90 consecutive amino acids of any of SEQ ID NO.:36, SEQ ID NO.:37, SEQ ID NO.:38, SEQ ID NO.:14, SEQ ID NO.:16, SEQ ID NO.:18 or SEQ ID NO.:20;
c) a light chain variable region which may comprise amino acids at least 90 consecutive amino acids of SEQ ID NO.:35 and a heavy chain variable region which may comprise at least 90 consecutive amino acids of any of SEQ ID NO.:36, SEQ ID NO.:37, SEQ ID NO.:38, SEQ ID NO.:14, SEQ ID NO.:16, SEQ ID NO.:18 or SEQ ID NO.:20;
d) a light chain variable region which may comprise at least 90 consecutive amino acids of SEQ ID NO.:8 and a heavy chain variable region which may comprise at least 90 consecutive amino acids of any of SEQ ID NO.:36, SEQ ID NO.:37, SEQ ID NO.:38, SEQ ID NO.:14, SEQ ID NO.:16, SEQ ID NO.:18 or SEQ ID NO.:20; or
e) a light chain variable region which may comprise at least 90 consecutive amino acids of SEQ ID NO.:10 and a heavy chain variable region which may comprise at least 90 consecutive amino acids of any of SEQ ID NO.:36, SEQ ID NO.:37, SEQ ID NO.:38, SEQ ID NO.:14, SEQ ID NO.:16, SEQ ID NO.:18 or SEQ ID NO.:20.

In accordance with a more specific embodiment of the invention, the light chain variable region may comprise at least 90 consecutive amino acids of SEQ ID NO.:8 or 10 and the heavy chain variable region may comprise at least 90 consecutive amino acids of SEQ ID NO.:14, 16, 18 or 20.

In accordance with an even more specific embodiment of the invention, the light chain variable region may be as set forth in SEQ ID NO.:8 or 10 and the heavy chain variable region may be as set forth in SEQ ID NO.:14, 16, 18 or 20.

More particularly, antibodies comprising the light chain variable region set fort in SEQ ID NO.: 8 and the heavy chain variable region set forth in SEQ ID NO.:14 are contemplated.

Other exemplary embodiments of the antibodies or antigen-binding fragments of the invention are those which may comprise a light chain variable region which may comprise at least 90 consecutive amino acids of any of SEQ ID Nos. 39, 40, 41, or 24.

As used herein the term “at least 90 consecutive amino acids of SEQ ID NO.:39” also includes the terms “at least 91, 92, 93, 94, 95, 96, 97, 98, 99, 100, 101, 102, 103, 104, 105, 106, 107, 108, 109, 110 111 or at least 112, consecutive amino acids”. The term “at least 90 consecutive amino acids of SEQ ID NO.:39” encompasses any possible sequence of at least 90 consecutive amino acids found in SEQ ID NO.:39 and especially those sequences which include the 3 CDRs of SEQ ID NO.:39, for example a sequence comprising amino acids 6 to 102, 11 to 106, 1 to 106, 3 to 95, 5 to 95 of SEQ ID NO.:39 and so on.

As used herein the term “at least 90 consecutive amino acids of SEQ ID NO.:40” also includes the terms “at least 91, 92, 93, 94, 95, 96, 97, 98, 99, 100, 101, 102, 103, 104, 105, 106, 107, 108, 109, 110, 111 or at least 112, consecutive amino acids”. The term “at least 90 consecutive amino acids of SEQ ID NO.:40” encompasses any possible sequence of at least 90 consecutive amino acids found in SEQ ID NO.:40 and especially those sequences which include the 3 CDRs of SEQ ID NO.:40, for example a sequence comprising amino acids 9 to 106, 10 to 101, 1 to 98, 3 to 99, 7 to 107 of SEQ ID NO.:40 and so on.

The terms “at least 90 consecutive amino acids of SEQ ID NO.:41” or “at least 90 consecutive amino acids of SEQ ID NO.:24” have similar meanings.

In accordance with the present invention, the antibody or antigen-binding fragment of the present invention may have, for example, a light chain variable region as set forth in SEQ ID NO.:24.

The antibody or antigen-binding fragment of the invention includes (or further includes) for example, a heavy chain variable region which may comprise at least 90 consecutive amino acids of any of SEQ ID NOs.:42, 43, 44 or 26.

As used herein the term “at least 90 consecutive amino acids of SEQ ID NO.:42” also includes the terms “at least 91, 92, 93, 94, 95, 96, 97, 98, 99, 100, 101, 102, 103, 104, 105, 106, 107, 108, 109, 110, 111, 112, 113, 114, 115, 116, 117 or at least 118 consecutive amino acids”. The term “at least 90 consecutive amino acids of SEQ ID NO.:42” encompasses any possible sequence of at least 90 consecutive amino acids found in SEQ ID NO.:42 and especially those sequences which include the 3 CDRs of SEQ ID NO.:42, such as, for example a sequence comprising amino acids 6 to 111, 1 to 106, 2 to 104, 5 to 106, 10 to 107 of SEQ ID NO.:42 and so on.

As used herein the term “at least 90 consecutive amino acids of SEQ ID NO.:43” also includes the terms “at least 91, 92, 93, 94, 95, 96, 97, 98, 99, 100, 101, 102, 103, 104, 105, 106, 107, 108, 109, 110, 111, 112, 113, 114, 115, 116, 117 or at least 118 consecutive amino acids”. The term “at least 90 consecutive amino acids of SEQ ID NO.:43” encompasses any possible sequence of at least 90 consecutive amino acids found in SEQ ID NO.:43 and especially those sequences which include the 3 CDRs of SEQ ID NO.:43, such as, for example a sequence comprising amino acids 3 to 107, 1 to 115, 1 to 110, 22 to 116, 20 to 115 of SEQ ID NO.:43 and so on.

The terms “at least 90 consecutive amino acids of SEQ ID NO.:44” or “at least 90 consecutive amino acids of SEQ ID NO.:26” has a similar meaning.

In accordance with the present invention, the antibody or antigen-binding fragment of the present invention may have, for example, a heavy chain variable region as set forth in SEQ ID NO.:28.

In accordance with the present invention the antibody or antigen-binding fragment may comprise, for example,
a) a light chain variable region which may comprise at least 90 consecutive amino acids of SEQ ID NO.:39 and a heavy chain variable region which may comprise at least 90 consecutive amino acids of any of SEQ ID NO.:42, SEQ ID NO.:43, SEQ ID NO.:44 or SEQ ID NO.:28;
b) a light chain variable region which may comprise at least 90 consecutive amino acids of SEQ ID NO.:40 and a heavy chain variable region which may comprise at least 90 consecutive amino acids of any of SEQ ID NO.:42, SEQ ID NO.:43, SEQ ID NO.:44 or SEQ ID NO.:28;
c) a light chain variable region which may comprise amino acids at least 90 consecutive amino acids of SEQ ID NO.:41 and a heavy chain variable region which may comprise at least 90 consecutive amino acids of any of SEQ ID NO.:42, SEQ ID NO.:43, SEQ ID NO.:44 or SEQ ID NO.:28 or;
d) a light chain variable region which may comprise at least 90 consecutive amino acids of SEQ ID NO.:24 and a heavy chain variable region which may comprise at least 90 consecutive amino acids of any of SEQ ID NO.:42, SEQ ID NO.:43, SEQ ID NO.:44 or SEQ ID NO.:28.

In accordance with a more specific embodiment of the invention, the light chain variable region may have at least 90 consecutive amino acids of SEQ ID NO.:24 and the heavy chain variable region may have at least 90 consecutive amino acids of SEQ ID NO.:28.

In accordance with an even more specific embodiment of the invention, the light chain variable region may be as set forth in SEQ ID NO.:24 and the heavy chain variable region may be as set forth in SEQ ID NO.:28.

Embodiments of the invention more particularly comprises an antibody or antigen-binding fragment selected from the group consisting of:
a. an antibody comprising a light chain having an amino acid sequence as set forth in SEQ ID NO.:7 and a heavy chain having an amino acid sequence as set forth in SEQ ID NO.:13 or an antigen-binding fragment thereof;
b. an antibody comprising a light chain having an amino acid sequence as set forth in SEQ ID NO.:7 and a heavy chain having an amino acid sequence as set forth in SEQ ID NO.:15 or an antigen-binding fragment thereof;
c. an antibody comprising a light chain having an amino acid sequence as set forth in SEQ ID NO.:7 and a heavy chain having an amino acid sequence as set forth in SEQ ID NO.:17 or an antigen-binding fragment thereof;
d. an antibody comprising a light chain having an amino acid sequence as set forth in SEQ ID NO.:7 and a heavy chain having an amino acid sequence as set forth in SEQ ID NO.:19 or an antigen-binding fragment thereof;
e. an antibody comprising a light chain having an amino acid sequence as set forth in SEQ ID NO.:7 and a heavy chain having an amino acid sequence as set forth in SEQ ID NO.:29 or an antigen-binding fragment thereof;
f. an antibody comprising a light chain having an amino acid sequence as set forth in SEQ ID NO.:7 and a heavy chain having an amino acid sequence as set forth in SEQ ID NO.:59 or an antigen-binding fragment thereof;
g. an antibody comprising a light chain having an amino acid sequence as set forth in SEQ ID NO.:7 and a heavy chain having an amino acid sequence as set forth in SEQ ID NO.:60 or an antigen-binding fragment thereof;
h. an antibody comprising a light chain having an amino acid sequence as set forth in SEQ ID NO.:7 and a heavy chain having an amino acid sequence as set forth in SEQ ID NO.:61 or an antigen-binding fragment thereof;
i. an antibody comprising a light chain having an amino acid sequence as set forth in SEQ ID NO.:9 and a heavy chain having an amino acid sequence as set forth in SEQ ID NO.:13 or an antigen-binding fragment thereof;
j. an antibody comprising a light chain having an amino acid sequence as set forth in SEQ ID NO.:9 and a heavy chain having an amino acid sequence as set forth in SEQ ID NO.:15 or an antigen-binding fragment thereof;
k. an antibody comprising a light chain having an amino acid sequence as set forth in SEQ ID NO.:9 and a heavy chain having an amino acid sequence as set forth in SEQ ID NO.:17 or an antigen-binding fragment thereof;
l. an antibody comprising a light chain having an amino acid sequence as set forth in SEQ ID NO.:9 and a heavy chain having an amino acid sequence as set forth in SEQ ID NO.:19 or an antigen-binding fragment thereof;
m. an antibody comprising a light chain having an amino acid sequence as set forth in SEQ ID NO.:9 and a heavy chain having an amino acid sequence as set forth in SEQ ID NO.:29 or an antigen-binding fragment thereof;
n. an antibody comprising a light chain having an amino acid sequence as set forth in SEQ ID NO.:9 and a heavy chain having an amino acid sequence as set forth in SEQ ID NO.:59 or an antigen-binding fragment thereof;
o. an antibody comprising a light chain having an amino acid sequence as set forth in SEQ ID NO.:9 and a heavy chain having an amino acid sequence as set forth in SEQ ID NO.:60 or an antigen-binding fragment thereof;
p. an antibody comprising a light chain having an amino acid sequence as set forth in SEQ ID NO.:9 and a heavy chain having an amino acid sequence as set forth in SEQ ID NO.:61 or an antigen-binding fragment thereof;
q. an antibody comprising a light chain having an amino acid sequence as set forth in SEQ ID NO.:23 and a heavy chain having an amino acid sequence as set forth in SEQ ID NO.:27 or an antigen-binding fragment thereof, and;
r. an antibody comprising a light chain having an amino acid sequence as set forth in SEQ ID NO.:23 and a heavy chain having an amino acid sequence as set forth in SEQ ID NO.:46 or an antigen-binding fragment thereof;
   wherein the amino acid sequence of the signal peptide of the light chain and/or heavy chain of any one or all of a. to r. is either present, absent or different.

Other embodiments of the invention comprises an antibody or antigen-binding fragment selected from the group consisting of:
a. an antibody comprising a light chain having an amino acid sequence as set forth in SEQ ID NO.:5 and a heavy chain having an amino acid sequence as set forth in SEQ ID NO.:13 or an antigen-binding fragment thereof;
b. an antibody comprising a light chain having an amino acid sequence as set forth in SEQ ID NO.:5 and a heavy chain having an amino acid sequence as set forth in SEQ ID NO.:15 or an antigen-binding fragment thereof;
c. an antibody comprising a light chain having an amino acid sequence as set forth in SEQ ID NO.:5 and a heavy chain having an amino acid sequence as set forth in SEQ ID NO.:17 or an antigen-binding fragment thereof;
d. an antibody comprising a light chain having an amino acid sequence as set forth in SEQ ID NO.:5 and a heavy chain having an amino acid sequence as set forth in SEQ ID NO.:19 or an antigen-binding fragment thereof;
e. an antibody comprising a light chain having an amino acid sequence as set forth in SEQ ID NO.:5 and a heavy chain having an amino acid sequence as set forth in SEQ ID NO.:29 or an antigen-binding fragment thereof;
f. an antibody comprising a light chain having an amino acid sequence as set forth in SEQ ID NO.:5 and a heavy chain having an amino acid sequence as set forth in SEQ ID NO.:59 or an antigen-binding fragment thereof;
g. an antibody comprising a light chain having an amino acid sequence as set forth in SEQ ID NO.:5 and a heavy chain having an amino acid sequence as set forth in SEQ ID NO.:60 or an antigen-binding fragment thereof;
h. an antibody comprising a light chain having an amino acid sequence as set forth in SEQ ID NO.:5 and a heavy chain having an amino acid sequence as set forth in SEQ ID NO.:61 or an antigen-binding fragment thereof;
i. an antibody comprising a light chain having an amino acid sequence as set forth in SEQ ID NO.:7 and a heavy chain having an amino acid sequence as set forth in SEQ ID NO.:11 or an antigen-binding fragment thereof;
j. an antibody comprising a light chain having an amino acid sequence as set forth in SEQ ID NO.:7 and a heavy chain having an amino acid sequence as set forth in SEQ ID NO.:30 or an antigen-binding fragment thereof;
k. an antibody comprising a light chain having an amino acid sequence as set forth in SEQ ID NO.:9 and a heavy chain having an amino acid sequence as set forth in SEQ ID NO.:11 or an antigen-binding fragment thereof; and
l. an antibody comprising a light chain having an amino acid sequence as set forth in SEQ ID NO.:9 and a heavy chain having an amino acid sequence as set forth in SEQ ID NO.:30 or an antigen-binding fragment thereof;
   wherein the amino acid sequence of the signal peptide of the light chain and/or heavy chain of any one or all of a. to l. is either present, absent or different.

The antibody or antigen-binding fragment of the present invention may have a light chain variable region and/or heavy chain variable region as described above and may further comprise amino acids of a constant region, such as, for example, amino acids of a constant region of a human antibody.

In an exemplary embodiment, the antibody or antigen-binding fragment of the present invention may comprise, for example, a human IgG1 constant region.

Anti-Siglec-15 antibodies of the IgG1 subtypes, which have, for example, an increase in activity of at least 10 fold in comparison with corresponding IgG2 subtypes or other subtypes) are particularly contemplated.

An increase in the potency of the IgG1-based anti-Siglec-15 antibody of at least 2, 3, 4, 5, 6, 7, 8, 9, 10, 20, 30, 40, 50, 60, 70, 80, 90, 95, 100 fold or more or an increase in its affinity of at least 2, 3, 4, 5, 6, 7, 8, 9, 10, 20, 30, 40, 50, 60, 70, 80, 90, 95, 100 fold or more may be particularly useful.

The increased in potency or affinity may be measured by the ability of the IgG1-based anti-Siglec-15 antibody to inhibit osteoclast differentiation or osteoclast activity in comparison with a different antibody subtype having identical or substantially identical CDRs or variable regions. In some circumstances, it may be possible to consider using an IgG1 antibody concentration as low as 10 ng/ml or 100 ng/ml for attempting to inhibit osteoclast differentiation and/or bone resorption in vitro. It may be understood herein that lower dosage of IgG1-based anti-Siglec-15 antibodies may achieve a desired therapeutic effect when compared, for example, with a corresponding IgG2-based anti-Siglec-15.

Particularly contemplated antibodies include those having a kappa light chain constant region and an IgG1 heavy chain constant region.

Antibodies and antigen-binding fragments of the invention include for example, monoclonal antibodies, polyclonal antibodies, human antibodies, humanized antibodies, chimeric antibodies having the amino acid sequence described herein. Human and humanized antibodies having the amino acid sequences identified herewith are particularly contemplated.

It is to be understood herein that the sequences of antibodies or antigen-binding fragments thereof made of a) a light chain variable region set forth in SEQ ID NO.:6 and a heavy chain variable region set forth in SEQ ID NO.:12 or b) a light chain variable region set forth in SEQ ID NO.:22 and a heavy chain variable region set forth in SEQ ID NO.: 26 are considered of mouse origin (i.e., a non-human antibody). Humanization of these antibodies would therefore be advantageous.

As indicated herein, humanization of a non-human antibody may be performed for example, by substitution of framework amino acids for corresponding amino acids of a natural human antibody. Substitutions are usually made in a manner that does not negatively affect antigen-binding.

In accordance with another exemplary embodiment of the invention, the antigen-binding fragment may be, for example, a scFv, a Fab, a Fab′ or a (Fab′)₂.

Variant antibodies or antigen-binding fragments encompassed by the present invention are those, which may comprise an insertion, a deletion or an amino acid substitution (conservative or non-conservative) but that has an activity similar to that of the original antibody. These variants may have at least one amino acid residue in its amino acid sequence removed and a different residue inserted in its place.

Sites of interest for substitutional mutagenesis include the hypervariable regions (CDRs), but modifications in the framework region or even in the constant region are also contemplated. Conservative substitutions may be made by exchanging an amino acid (of a CDR, variable chain, antibody, etc.) from one of the groups listed below (group 1 to 6) for another amino acid of the same group.

Generally, mutations in the CDRs may have a greater impact on the antigen-binding activity of the antibody or antigen-binding fragment than mutations in the framework region. Mutation in the framework region may be performed to increase the “humanness” of the antibody. Variant antibody or antigen-binding fragments that are encompassed by the present invention are those which have a substantially identical antigen-binding capacity (including similar, identical, or slightly less) to those presented herein or have a better antigen-binding capacity than those presented herein.

Other exemplary embodiment of conservative substitutions are shown in Table 1A under the heading of “preferred substitutions”. If such substitutions result in a undesired property, then more substantial changes, denominated “exemplary substitutions” in Table 1A, or as further described below in reference to amino acid classes, may be introduced and the products screened.

It is known in the art that variants may be generated by substitutional mutagenesis and retain the biological activity of the polypeptides of the present invention. These variants have at least one amino acid residue in the amino acid sequence removed and a different residue inserted in its place. For example, one site of interest for substitutional mutagenesis may include a site in which particular residues obtained from various species are identical. Examples of substitutions identified as “conservative substitutions” are shown in Table 1A. If such substitutions result in a change not desired, then other type of substitutions, denominated “exemplary substitutions” in Table 1A, or as further described herein in reference to amino acid classes, are introduced and the products screened.

Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(group 1) hydrophobic: norleucine, methionine (Met), Alanine (Ala), Valine (Val), Leucine (Leu), Isoleucine (Ile)
(group 2) neutral hydrophilic: Cysteine (Cys), Serine (Ser), Threonine (Thr)
(group 3) acidic: Aspartic acid (Asp), Glutamic acid (Glu)
(group 4) basic: Asparagine (Asn), Glutamine (Gin), Histidine (His), Lysine (Lys), Arginine (Arg)
(group 5) residues that influence chain orientation: Glycine (Gly), Proline (Pro); and
(group 6) aromatic: Tryptophan (Trp), Tyrosine (Tyr), Phenylalanine (Phe)
   Non-conservative substitutions will entail exchanging a member of one of these classes for another.

TABLE 1A
Amino acid substitution

| Original | | Conservative | Semi-conservative |
|---|---|---|---|
| residue | Exemplary substitution | substitution | substitution |
| Ala (A) | Val, Leu, Ile | Val | N, V, P, (C) |
| Arg (R) | Lys, Gln, Asn | Lys | S, T, E, D, A |
| Asn (N) | Gln, His, Lys, Arg, Asp | Gln | K, R |
| Asp (D) | Glu, Asn | Glu | K, R, H, A |
| Cys (C) | Ser, Ala | Ser | F, G |
| Gln (Q) | Asn; Glu | Asn | M, L, K, R |
| Glu (E) | Asp, Gln | Asp | K, R, H, A |
| Gly (G) | Ala | Ala | — |
| His (H) | Asn, Gln, Lys, Arg, | Arg | L, M, A, (C) |
| Ile (I) | Leu, Val, Met, Ala, Phe, | Leu | F, Y, W, G, (C) |
| | norleucine | | |
| Leu (L) | Norleucine, Ile, Val, Met, | Ile | F, Y, W, H, (C) |
| | Ala, Phe | | |
| Lys (K) | Arg, Gln, Asn | Arg | Q, N, S, T, D, E, A |
| Met (M) | Leu, Phe, Ile | Leu | Q, F, Y, W, (C), (R) |
| | | | (K), (E) |
| Phe (F) | Leu, Val, Ile, Ala, Tyr | Tyr | I, V, (C) |
| Pro (P) | Ala | Ala | A, (C), (D), (E), F, H |
| | | | (K), L, M, N, Q, (R), S |
| | | | T, W, Y |
| Ser (S) | Thr | Thr | D, E, R, K |
| Thr (T) | Ser | Ser | D, E, R, K, I |
| Trp (W) | Tyr, Phe | Tyr | L, M, I, V, (C) |
| Tyr (Y) | Trp, Phe, Thr, Ser | Phe | L, M, I, V, (C) |
| Val (V) | Ile, Leu, Met, Phe, Ala, | Leu | P, (C) |
| | norleucine | | |

Changing from A, F, H, I, L, M, P, V, W, or Y to C is semi-conservative if the cysteine remains as a free thiol. Changing from M to E, R, K is semi-conservative if the ionic tip of the new side group may reach the protein surface while the methylene groups make hydrophobic contact. Changing from P to one of K, R, E or D is semi-conservative if the side group is on or near the surface of the protein.

Variation in the amino acid sequence of the variant antibody or antigen-binding fragment may include an amino acid addition, deletion, insertion, substitution etc., one or more modification in the backbone or side-chain of one or more amino acid, or an addition of a group or another molecule to one or more amino acids (side-chains or backbone).

Variant antibody or antigen-binding fragment may have substantial sequence similarity and/or sequence identity in its amino acid sequence in comparison with that of the original antibody or antigen-binding fragment amino acid sequence. The degree of similarity between two sequences is based upon the percentage of identities (identical amino acids) and of conservative substitution.

Generally, the degree of similarity and identity between variable chains has been determined herein using the Blast2 sequence program (Tatiana A. Tatusova, Thomas L. Madden (1999), “Blast 2 sequences—a new tool for comparing protein and nucleotide sequences”, FEMS Microbiol Lett. 174:247-250) using default settings, i.e., blastp program, BLOSUM62 matrix (open gap 11 and extension gap penalty 1; gapx dropoff 50, expect 10.0, word size 3) and activated filters.

Percent identity may therefore be indicative of amino acids which are identical in comparison with the original peptide and which may occupy the same or similar position.

Percent similarity may be indicative of amino acids that are identical and those which are replaced with conservative amino acid substitution in comparison with the original peptide at the same or similar position.

Variants (i.e., analogues) of the present invention (including VL variants, VH variants, CDR variants, antibody variants, polypeptide variants, etc.) therefore comprise those which may have at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with an original sequence or a portion of an original sequence.

In accordance with the present invention, a SEQ ID NO.:2 variant includes a polypeptide having a region at least 80% identical with amino acids 49-165 or with amino acids 20 to 259 of SEQ ID NO.:2. Variants of SEQ ID NO.:2 also include polypeptides having at least 80% sequence identity with SEQ ID NO.:2. Preferred variants of SEQ ID NO.:2 includes those that are able to inhibit osteoclast differentiation and/or bone resorption. Such variants may be identified, for example, by testing their osteoclast differentiation and/or bone resorption activity in vitro or in vivo. Examples of methods or assays that may be used to test the activity of Siglec-15 variants are described herein and have been provided in international application No. PCT/CA2007/001134. It is to be understood that the osteoclasts used to perform the assays described herein may originate, for example, preferably from human but also from mouse, rat or monkey (e.g., cynomolgus). Preferred variants of SEQ ID NO.:2 may include, for example, those where an epitope comprising arginine 99 (R99) of SEQ ID NO.:2 is preserved.

Exemplary embodiments of variants are those having at least 81% sequence identity to a sequence described herein and 81%, 82%, 83%, 84%, 85%, 86%, 87%, 88%, 89%, 90%, 91%, 92%, 93%, 94%, 95%, 96%, 97%, 98%, 99% or 100% sequence similarity with an original sequence or a portion of an original sequence.

Other exemplary embodiments of variants are those having at least 82% sequence identity to a sequence described herein and 82%, 83%, 84%, 85%, 86%, 87%, 88%, 89%, 90%, 91%, 92%, 93%, 94%, 95%, 96%, 97%, 98%, 99% or 100% sequence similarity with an original sequence or a portion of an original sequence.

Further exemplary embodiments of variants are those having at least 85% sequence identity to a sequence described herein and 85%, 86%, 87%, 88%, 89%, 90%, 91%, 92%, 93%, 94%, 95%, 96%, 97%, 98%, 99% or 100% sequence similarity with an original sequence or a portion of an original sequence.

Other exemplary embodiments of variants are those having at least 90% sequence identity to a sequence described herein and 90%, 91%, 92%, 93%, 94%, 95%, 96%, 97%, 98%, 99% or 100% sequence similarity with an original sequence or a portion of an original sequence.

Additional exemplary embodiments of variants are those having at least 95% sequence identity to a sequence described herein and 95%, 96%, 97%, 98%, 99% or 100% sequence similarity with an original sequence or a portion of an original sequence.

Yet additional exemplary embodiments of variants are those having at least 97% sequence identity to a sequence described herein and 97%, 98%, 99% or 100% sequence similarity with an original sequence or a portion of an original sequence.

For a purpose of concision the applicant provides herein a Table 1B illustrating exemplary embodiments of individual variants encompassed by the present invention and comprising the specified % sequence identity and % sequence similarity. Each “X” is to be construed as defining a given variant.

TABLE 1B

| | | Percent (%) sequence identity | | | | | | | | | | | | | | | | | | | | |
|---|---|---|---|---|---|---|---|---|---|---|---|---|---|---|---|---|---|---|---|---|---|---|
| | | 80 | 81 | 82 | 83 | 84 | 85 | 86 | 87 | 88 | 89 | 90 | 91 | 92 | 93 | 94 | 95 | 96 | 97 | 98 | 99 | 100 |
| Percent | 80 | X | | | | | | | | | | | | | | | | | | | | |
| (%) | 81 | X | X | | | | | | | | | | | | | | | | | | | |
| sequence | 82 | X | X | X | | | | | | | | | | | | | | | | | | |
| similarity | 83 | X | X | X | X | | | | | | | | | | | | | | | | | |
| | 84 | X | X | X | X | X | | | | | | | | | | | | | | | | |
| | 85 | X | X | X | X | X | X | | | | | | | | | | | | | | | |
| | 86 | X | X | X | X | X | X | X | | | | | | | | | | | | | | |
| | 87 | X | X | X | X | X | X | X | X | | | | | | | | | | | | | |
| | 88 | X | X | X | X | X | X | X | X | X | | | | | | | | | | | | |
| | 89 | X | X | X | X | X | X | X | X | X | X | | | | | | | | | | | |
| | 90 | X | X | X | X | X | X | X | X | X | X | X | | | | | | | | | | |
| | 91 | X | X | X | X | X | X | X | X | X | X | X | X | | | | | | | | | |
| | 92 | X | X | X | X | X | X | X | X | X | X | X | X | X | | | | | | | | |
| | 93 | X | X | X | X | X | X | X | X | X | X | X | X | X | X | | | | | | | |
| | 94 | X | X | X | X | X | X | X | X | X | X | X | X | X | X | X | | | | | | |
| | 95 | X | X | X | X | X | X | X | X | X | X | X | X | X | X | X | X | | | | | |
| | 96 | X | X | X | X | X | X | X | X | X | X | X | X | X | X | X | X | X | | | | |
| | 97 | X | X | X | X | X | X | X | X | X | X | X | X | X | X | X | X | X | X | | | |
| | 98 | X | X | X | X | X | X | X | X | X | X | X | X | X | X | X | X | X | X | X | | |
| | 99 | X | X | X | X | X | X | X | X | X | X | X | X | X | X | X | X | X | X | X | X | |
| | 100 | X | X | X | X | X | X | X | X | X | X | X | X | X | X | X | X | X | X | X | X | X |

As used herein, the term “identical” means that a sequence share 100% sequence identity with another sequence.

As used herein, the term “substantially identical” means that a sequence share 70%, 75%, 80%, 81%, 82%, 83%, 84%, 85%, 86%, 87%, 88%, 89%, 90%, 91%, 92%, 93%, 94%, 95%, 96%, 97%, 98%, 99% sequence identity with another sequence or a portion of another sequence.

The present invention encompasses CDRs, light chain variable domains, heavy chain variable domains, light chains, heavy chains, antibodies and/or antigen-binding fragments which comprise at least 70% identity (including any range between 70% and 99%) with the sequence described herein.

### Antibody Conjugates

Although it is not always necessary, for detection or therapeutic purposes, the antibody or antigen-binding fragment of the present invention (including antibody variants) may be conjugated with a detectable moiety (i.e., for detection or diagnostic purposes) or with a therapeutic moiety (for therapeutic purposes).

For detection purposes, an unconjugated antibody (primary antibody) may be used for binding to the antigen and a secondary antibody carrying a detectable moiety and capable of binding to the primary antibody may be added. However, as indicated above, the anti-Siglec-15 antibody may be conjugated with a detectable label and as such a secondary antibody may not be necessary,

A “detectable moiety” may comprise, for example, a moiety detectable by spectroscopic, photochemical, biochemical, immunochemical, chemical and/or other physical means. A detectable moiety may be coupled either directly and/or indirectly (for example via a linkage, such as, without limitation, a DOTA or NHS linkage) to antibodies and antigen-binding fragments thereof of the present invention using methods well known in the art. A wide variety of detectable moieties may be used, with the choice depending on the sensitivity required, ease of conjugation, stability requirements and available instrumentation. A suitable detectable moiety include, but is not limited to, a fluorescent label, a radioactive label (for example, without limitation, ¹²⁵I, In¹¹¹, Tc⁹⁹, I¹³¹ and including positron emitting isotopes for PET scanner, etc), a nuclear magnetic resonance active label, a luminescent label, a chemiluminescent label, a chromophore label, an enzyme label (for example and without limitation horseradish peroxidase, alkaline phosphatase, etc.), quantum dots and/or a nanoparticle. Detectable moiety may cause and/or produce a detectable signal thereby allowing for a signal from the detectable moiety to be detected.

In another exemplary embodiment of the invention, the antibody or antigen-binding fragment thereof may be coupled (modified) with a therapeutic moiety (e.g., drug, cytotoxic moiety).

In some instances, for therapeutic purposes, an unconjugated antibody may by itself be capable of sequestering the antigen, may block an important interaction between the antigen and another binding partner, may recruit effector cells, etc. However, as indicated above, the antibody may be conjugated with a therapeutic moiety.

In an exemplary embodiment, the antibodies and antigen-binding fragments may comprise a chemotherapeutic or cytotoxic agent. For example, the antibody and antigen-binding fragments may be conjugated to the chemotherapeutic or cytotoxic agent. Such chemotherapeutic or cytotoxic agents include, but are not limited to, Yttrium-90, Scandium-47, Rhenium-186, Iodine-131, Iodine-125, and many others recognized by those skilled in the art (e.g., lutetium (e.g., Lu¹⁷⁷), bismuth (e.g., Bi²¹³), copper (e.g., Cu⁶⁷)). In other instances, the chemotherapeutic or cytotoxic agent may be comprised of, among others known to those skilled in the art, 5-fluorouracil, adriamycin, irinotecan, auristatins, taxanes, pseudomonas endotoxin, ricin, calicheamicin, amatoxin (e.g., alpha-amanintin), pyrrolobenzodiazepine (PBD) and other toxins. Exemplary cytotoxic agents may particularly comprise an agent, which is capable of killing non-proliferating cells.

The antibody or antigen-binding fragment of the present invention may especially be conjugated with agents targeting DNA. Exemplary embodiments of agents targeting DNA includes for example, alkylating agents such as duocarmycins and duocarmycin derivatives such as adozelesin, bizelesin, carzelesin etc. Other exemplary embodiments of agents targeting DNA includes for example, calicheamicin, esperamicin and derivatives (see compounds disclosed for example in U.S. Pat. Nos. 5,264,586, 5,108,192, 4,970,198, 5,037,651, 5,079,233, 4,675,187, 4,539,203, 4,554,162, 4,837,206 and US2007213511, the entire content of each document is incorporated herein by reference).

A particular embodiment of the invention includes for example, an antibody or antigen-binding fragment disclosed herein conjugated with duocarmycin. Another particular embodiment of the invention includes for example, an antibody or antigen-binding fragment disclosed herein conjugated with calicheamicin.

Alternatively, in order to carry out the methods of the present invention and as known in the art, the antibody or antigen-binding fragment of the present invention (conjugated or not) may be used in combination with a second molecule (e.g., a secondary antibody, etc.) which is able to specifically bind to the antibody or antigen-binding fragment of the present invention and which may carry a desirable detectable, diagnostic or therapeutic moiety.

Pharmaceutical Compositions of the Antibodies and their Use

Pharmaceutical compositions of the antibodies are also encompassed by the present invention. The pharmaceutical composition may comprise an antibody or an antigen-binding fragment and may also contain a pharmaceutically acceptable carrier. The antibody or antigen-binding fragment thereof may conjugated with a therapeutic agent (e.g., toxin) or not (e.g., unconjugated).

Other aspects of the invention relate to a composition which may comprise the antibody or antigen-binding fragment described herein and a carrier.

In addition to the active ingredients, a pharmaceutical composition may contain pharmaceutically acceptable carriers comprising water, PBS, salt solutions, gelatins, oils, alcohols, and other excipients and auxiliaries that facilitate processing of the active compounds into preparations that may be used pharmaceutically. In other instances, such preparations may be sterilized.

As used herein, “pharmaceutical composition” usually comprises therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal, oral, vaginal, rectal routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein “pharmaceutically acceptable carrier” or “pharmaceutical carrier” are known in the art and include, but are not limited to, 0.01-0.1 M or 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's orfixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic compositions described above may be applied to any subject in need of such therapy, including, but not limited to, mammals such as dogs, cats, cows, horses, rabbits, monkeys, and humans.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

Pharmaceutical compositions of the present invention may further comprise for example, at least one drug member selected from the group consisting of bisphosphonates, active vitamin D3, calcitonin and derivatives thereof, hormone preparations such as estradiol, SERMs (selective estrogen receptor modulators), ipriflavone, vitamin K2 (menatetrenone), calcium preparations, PTH (parathyroid hormone) preparations, nonsteroidal anti-inflammatory agents, soluble TNF receptor preparations, anti-TNF-alpha antibodies or functional fragments of the antibodies, anti-PTHrP (parathyroid hormone-related protein) antibodies or functional fragments of the antibodies, IL-1 receptor antagonists, anti-IL-6 receptor antibodies or functional fragments of the antibodies, anti-RANKL antibodies or functional fragments of the antibodies and OCIF (osteoclastogenesis inhibitory factor).

The term “treatment” for purposes of this disclosure refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

In certain instances, the anti-Siglec-15 antibodies and antigen-binding fragment thereof may be administered concurrently in combination with other treatments given for the same condition. As such, the antibodies may be administered with anti-resorptives (e.g., bisphosphonates) that are known to those skilled in the art. In other instances, the anti-Siglec-15 antibodies and immunologically functional fragments therein may be administered with other therapeutic antibodies (e.g., anti-RANKL).

The antibodies may be sold in kits, provided in a vial suitable for transport and/or storage and comprising a package insert with information concerning suitable treatment of patients.

### Method of Treatment

Methods of treatment encompassed by the present invention include administration of the antibody or antigen-binding fragment (e.g., in the form of a pharmaceutical composition) to a desired target population.

Prior to administration, the target population may be selected for example by screening for the presence of a genetic mutation or dysfunction or phenotype associated with osteogenesis imperfecta type VI. Osteogenesis imperfecta type VI may be distinguished from the other types of osteogenesis imperfect based on clinical criteria outlined below.

The target population may mainly be composed of children and infants. These children or infants may have received a diagnosis of brittle bone disease.

In some instance, patients may also be treated with another drug. Such drug may include for example, bisphosphonates.

In accordance with the osteogenesis imperfecta foundation, the general features of Type I to VIII of OI are as follows:

### Type I

Most common and mildest type of OI.
Bones fracture easily. Most fractures occur before puberty.
Normal or near-normal stature.
Loose joints and muscle weakness.
Sclera (whites of the eyes) usually have a blue, purple, or gray tint.
Triangular face.
Tendency toward spinal curvature.
Bone deformity absent or minimal.
Brittle teeth possible.
Hearing loss possible, often beginning in early 20s or 30s.
Collagen structure is normal, but the amount is less than normal.

### Type II

Most severe form.
Frequently lethal at or shortly after birth, often due to respiratory problems.
Numerous fractures and severe bone deformity.
Small stature with underdeveloped lungs.
Tinted sclera.
Collagen improperly formed.

### Type III

Bones fracture easily. Fractures often present at birth, and x-rays may reveal healed fractures that occurred before birth.
Short stature.
Sclera have a blue, purple, or gray tint.
Loose joints and poor muscle development in arms and legs.
Barrel-shaped rib cage.
Triangular face.
Spinal curvature.
Respiratory problems possible.
Bone deformity, often severe.
Brittle teeth possible.
Hearing loss possible.
Collagen improperly formed.

### Type IV

Between Type I and Type III in severity.
Bones fracture easily. Most fractures occur before puberty.
Shorter than average stature.
Sclera are white or near-white (i.e. normal in color).
Mild to moderate bone deformity.
Tendency toward spinal curvature.
Barrel-shaped rib cage.
Triangular face.
Brittle teeth possible.
Hearing loss possible.
Collagen improperly formed.

By studying the appearance of OI bone under the microscope, investigators noticed that some people who are clinically within the Type IV group had a distinct pattern to their bone. When they reviewed the full medical history of these people, they found that groups had other features in common. They named these groups Types V and VI OI. People in these two groups do not appear to have mutations in the type I collagen genes.

### Type V

Clinically similar to Type IV in appearance and symptoms of OI.
A dense band seen on x-rays adjacent to the growth plate of the long bones.
Unusually large calluses (hypertrophic calluses) at the sites of fractures or surgical procedures. (A callus is an area of new bone that is laid down at the fracture site as part of the healing process.
Calcification of the membrane between the radius and ulna (the bones of the forearm). This leads to restriction of forearm rotation.
White sclera.
Normal teeth.
Bone has a “mesh-like” appearance when viewed under the microscope.
Dominant inheritance pattern

### Type VI

Clinically similar to Type IV in appearance and symptoms of OI.
The alkaline phosphatase (an enzyme linked to bone formation) activity level is slightly elevated in OI Type VI. This can be determined by a blood test.
Bone has a distinctive “fish-scale” appearance when viewed under the microscope.
Diagnosed by bone biopsy.
Whether this form is inherited in a dominant or recessive manner is unknown, but researchers believe the mode of inheritance is most likely recessive.
Eight people with this type of OI have been identified.

### Recessive Forms of OI

After years of research, two forms of OI that are inherited in a recessive manner were discovered in 2006. Both types are caused by genes that affect collagen formation. These forms provide information for people who have severe or moderately severe OI but who do not have a primary collagen mutation.

### Type VII

The first described cases resemble Type IV OI in many aspects of appearance and symptoms.
In other instances the appearance and symptoms are similar to Type II lethal OI, except infants had white sclera, a small head and a round face.
Short stature.
Short humerus (arm bone) and short femur (upper leg bone)
Coxa vera is common (the acutely angled femur head affects the hip socket).
Results from recessive inheritance of a mutation to the CRTAP (cartilage-associated protein) gene. Partial function of CRTAP leads to moderate symptoms while total absence of CRTAP was lethal in all 4 identified cases.

### Type VIII

Resembles lethal Type II or Type III OI in appearance and symptoms except that infants have white sclera.
Severe growth deficiency.
Extreme skeletal under mineralization.
Caused by a deficiency of P3H1 (Prolyl 3-hydroxylase 1) due to a mutation to the LEPRE1 gene.

### EXAMPLES

### Example 1

This example describes the ability of an antibody targeting Siglec-15 to restore the bone loss that is observed in a mouse model of osteogenesis imperfecta type VI. Recently, the knock-out of SERPINF1 in mice was described and the resulting phenotype in these animals greatly mimics the disease in humans (Bogan et al. 2013). In this report, the skeletal phenotype was characterized. Micro-computed tomography (μCT) and quantitative bone histomorphometry in femurs of mature Pedf null mutants revealed reduced trabecular bone volume and the accumulation of unmineralized bone matrix. It was also found that the bones were more brittle than controls. In vitro, osteoblasts from Pedf null mice exhibited enhanced mineral deposition as assessed by Alizarin Red staining and an increased mineral:matrix ratio. The findings in this mouse model mimic the principal structural and biochemical features of bone observed in humans with OI type VI and consequently provide a useful model with which to further investigate putative bone-targeted agents.

Three (3) groups of SERPINF1 null mice, each consisting of 8 males, were assigned to this study and treated as described in the paragraph and Table 2 below. Group 1 received twice weekly by intraperitoneal (IP) injections of the vehicle, PBS. Groups 2 and 3 were treated twice weekly with IP injections of a mouse IgG antibody targeting mouse Siglec-15 (anti-Siglec-15 that is capable of binding to mouse Siglec-15 and inhibiting differentiation of mouse osteoclasts), at a dose of 3 mg/kg and 10 mg/kg, respectively.

TABLE 2
Study Design

| | | Dose | Dose | Dose | | | |
|---|---|---|---|---|---|---|---|
| Group | | Level | Concentration | Volume | | | *Animal |
| No. | Treatment | (mg/kg) | (mg/mL) | (mL/kg) | Route | Frequency | ID |
| 1 | PBS | 0 | 1 | 10 | IP | q3d | 1001-1008 |
| 2 | Anti- | 3 | 0.3 | 10 | IP | q3d | 2001-2008 |
| | Siglec15 | | | | | | |
| 3 | Anti- | 10 | 1 | 10 | IP | q3d | 3001-3008 |
| | Siglec15 | | | | | | |

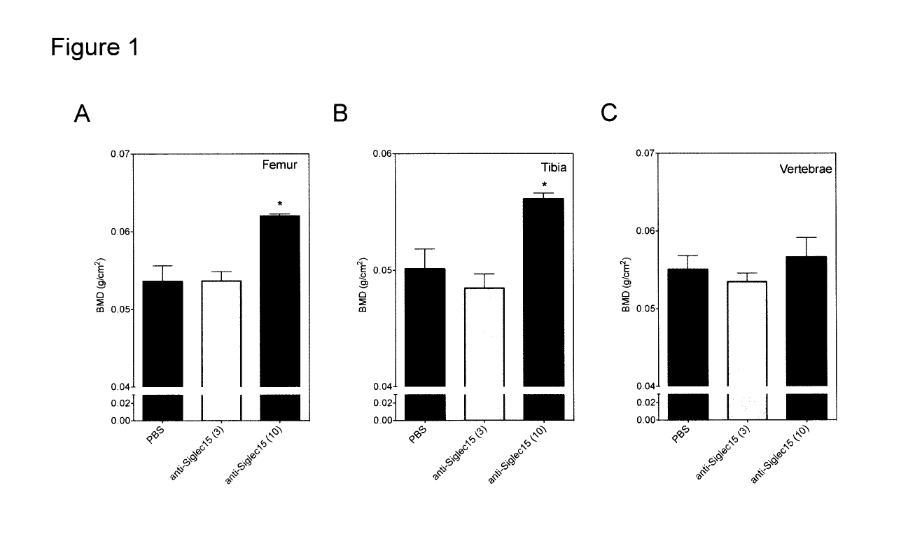

Results of FIGS. 1-4 were generated from data of 6 mice from the PBS control group and data of 3 mice from each of the anti-Siglec15-treated groups

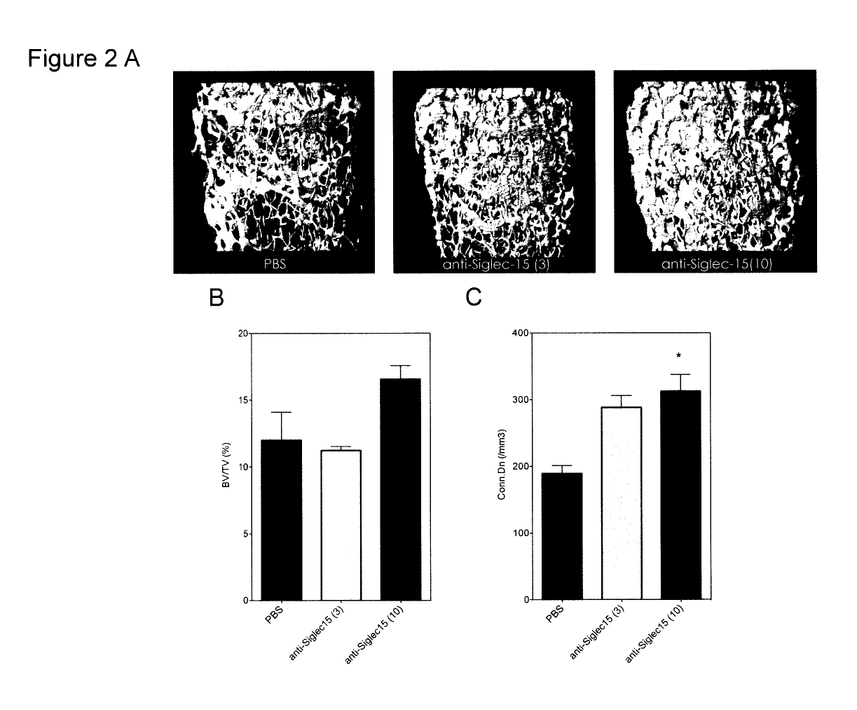

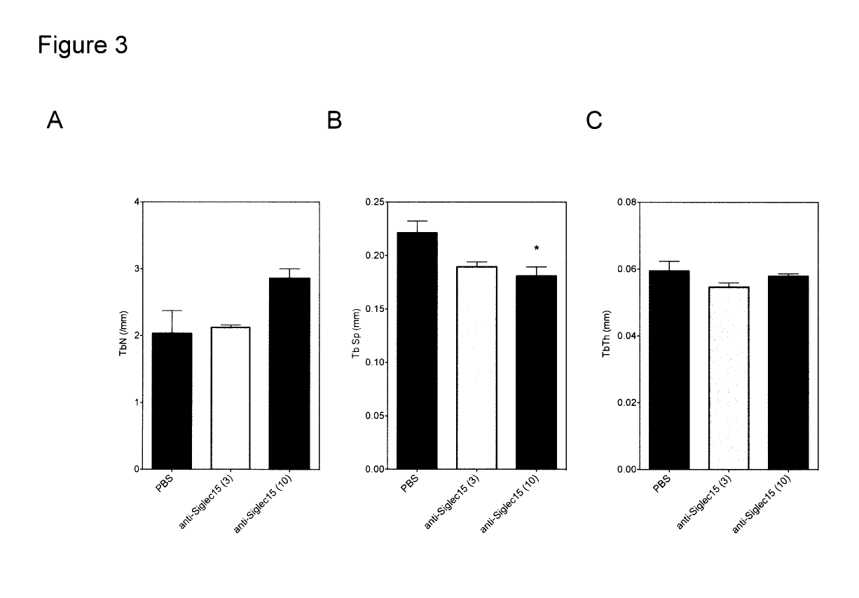

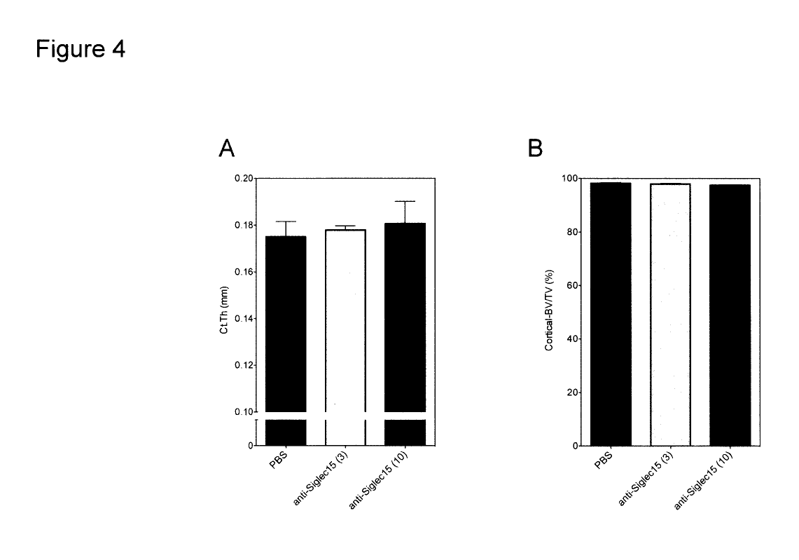

The day of dosing was defined as Day 1 and treatments were given for a total of 8 weeks. On Day 50, six (6) days before the end of the study, all animals received a first dose 10 mg/kg of calcein, a bone labeling fluorescent dye, administered IP. Three (3) days later, on Day 53, a second dose of calcein was administered. All animals were sacrificed on Day 56, three (3) days following the second injection of calcein. Left femur, left tibia and L1-L3 lumbar vertebrae were harvested and fixed in periodate-lysine-paraformaldehyde solution (PLP) for 24 hrs at 4° C., rinsed three times with 1×PBS pH 7.4, transferred to PBS and stored at 4° C. The bones scanned using a PIXImus Densitometer (GE Medical Systems) to determine the bone mineral density (BMD) of the femurs, the tibias and the vertebrae. Analysis of the bone microarchitecture and three-dimensional images of the distal femur were generated with a SkyScan high-resolution microCT (SKyScan Inc., Kontich, Belgium). As demonstrated in FIG. 1, animals treated with 10 mg/kg anti-Siglec-15 showed a significant increase in bone mineral density in the long bones, namely femur (FIG. 1A) and tibia (FIG. 1B), but no differences were observed in lumbar vertebrae (FIG. 1C). Treatment of SERPINF1 null animals with 3 mg/kg anti-Siglec-15 has almost no effect on the BMD at any of the sites analysed. MicroCT analysis demonstrated that animals treated with 10 mg/kg anti-Siglec-15 exhibited an increase in trabecular bone volume in the distal femur compared to vehicle treated animals (FIG. 2A). However, the trabecular connective density in the distal femur was increased by both treatment with 3 mg/kg and 10 mg/kg anti-Siglec-15, as shown in FIG. 2B. Moreover, trabecular number was increased with 10 mg/kg anti-Siglec-15 treatment (FIG. 3A) and the trabecular separation was correspondingly and significantly reduced compared to vehicle-treated animals (FIG. 3B). The trabecular thickness was unaffected by treatment with anti-Siglec-15 (FIG. 3C). Cortical parameters were similar in all groups where no change was observed in cortical thickness or cortical bone volume (FIG. 4B).

Taken together, these results demonstrate the ability of antibodies targeting Siglec-15 to increase the bone volume in a representative model of Osteogenesis Imperfecta Type VI, a disease for which patients have very few options.

### REFERENCES

The entire content of the references listed in the present application is incorporated herein by reference.
Stuible M. et al., “Anti-Siglec-15 antibodies” international application No. PCT/CA2013/000646 published on Jan. 23, 2014 under No. WO2014/012165
Sooknanan, R. R. et al., “Polynucleotides and polypeptide sequences involved in the process of bone remodelling”, international application No. PCT/CA2007/000210 (published on Aug. 23, 2007 under No. WO2007/093042).
Tremblay, G. B. et al. “Siglec 15 Antibodies in Treating Bone Loss-Related Disease”, international application No. PCT/CA2010/001586 (published on Apr. 14, 2011 under No. WO2011/041894);
Hiruma Y. et al., “Antibody Targeting Osteoclast-Related Protein Siglec-15”; U.S. Ser. No. 12/677,621 published on Aug. 19, 2010 under US2010/0209428A1;
Hiruma Y. et al., “Anti-Siglec-15 Antibody”, U.S. Ser. No. 13/143,253 published on Nov. 3, 2011 under No. US2011/0268733A1;
Hiruma Y. et al., “Novel Anti-Siglec15 Antibody”, international appl. No, PCT/JP2013/059653 published on Oct. 13, 2013 under No. WO/2013/147212.
Hiruma Y. et al., “Anti-Siglec15 Antibody with modified CDR”, international appl. No, PCT/JP2013/059654 published on Oct. 13, 2013 under No. WO/2013/147213.
Watanabe, I. et al., “Antibody Targeting Osteoclast-Related Protein Siglec-15”, international application No. PCT/EP2011/005219 published on Apr. 12, 2012 under No. WO2012045481A2.
Angata, T., T. Hayakawa, M. Yamanaka, A. Varki, and M. Nakamura. 2006. Discovery of Siglec-14, a novel sialic acid receptor undergoing concerted evolution with Siglec-5 in primates. FASEB J 20:1964-1973.
Angata, T., Y. Tabuchi, K. Nakamura, and M. Nakamura. 2007. Siglec-15: an immune system Siglec conserved throughout vertebrate evolution. Glycobiology 17:838-846.
Baron, R., S. Ferrari, and R. G. Russell. 2011. Denosumab and bisphosphonates: different mechanisms of action and effects. Bone 48:677-692.
Blasius, A. L., M. Cella, J. Maldonado, T. Takai, and M. Colonna. 2006. Siglec-H is an IPC-specific receptor that modulates type I IFN secretion through DAP12. Blood 107:2474-2476.
Blasius, A. L., and M. Colonna. 2006. Sampling and signaling in plasmacytoid dendritic cells: the potential roles of Siglec-H. Trends Immunol 27:255-260.
Bogan et al. (2013) A Mouse Model for Human Osteogenesis Imperfecta Type VI. J. Bone Miner. Res. 28:1531-1536
Bonifacino, J. S., and L. M. Traub. 2003. Signals for sorting of transmembrane proteins to endosomes and lysosomes. Annu Rev Biochem 72:395-447.
Boyle, W. J., W. S. Simonet, and D. L. Lacey. 2003. Osteoclast differentiation and activation. Nature 423:337-342.
Buckley, K. A. et al., Human Osteoclast Culture from Peripheral Blood Monocytes: Phenotypica Characterization and Quantitation of Resorption,” Methods in Molecular Medicine, vol. 107: Human Cell Culture Protocols, Second edition, 2005.
Collin-Osdoby P. et al., RANKL-Mediated Osteoclast Formation from Murine RAW 264.7 Cells,” Methods in Molecular Medicine, Vol. 80: Bone Research Protocols, 2003.
Crocker, P. R., J. C. Paulson, and A. Varki. 2007. Siglecs and their roles in the immune system. Nat Rev Immunol 7:255-266.
Crocker, P. R., and P. Redelinghuys. 2008. Siglecs as positive and negative regulators of the immune system. Biochem Soc Trans 36:1467-1471.
Hiruma, Y., T. Hirai, and E. Tsuda. 2011. Siglec-15, a member of the sialic acid-binding lectin, is a novel regulator for osteoclast differentiation. Biochem Biophys Res Commun 409:424-429.
Homan et al. (2011) Mutations in SERPINF1 Cause Osteogenesis Imperfecta Type VI. J. Bone Miner. Res. 26:2798-2803.
Humphrey, M. B., M. R. Daws, S. C. Spusta, E. C. Niemi, J. A. Torchia, L. L. Lanier, W. E. Seaman, and M. C. Nakamura. 2006. TREM2, a DAP12-associated receptor, regulates osteoclast differentiation and function. J Bone Miner Res 21:237-245.
Ishida-Kitagawa, N., K. Tanaka, X. Bao, T. Kimura, T. Miura, Y. Kitaoka, K. Hayashi, M. Sato, M. Maruoka, T. Ogawa, J. Miyoshi, and T. Takeya. 2012. Siglec-15 regulates the formation of functional osteoclasts in concert with DNAX-activating protein of 12 KDa (DAP12). J Biol Chem
Kaifu, T., J. Nakahara, M. Inui, K. Mishima, T. Momiyama, M. Kaji, A. Sugahara, H. Koito, A. Ujike-Asai, A. Nakamura, K. Kanazawa, K. Tan-Takeuchi, K. Iwasaki, W. M. Yokoyama, A. Kudo, M. Fujiwara, H. Asou, and T. Takai. 2003. Osteopetrosis and thalamic hypomyelinosis with synaptic degeneration in DAP12-deficient mice. J Clin Invest 111:323-332.
Lanier, L. L. 2009. DAP10- and DAP12-associated receptors in innate immunity. Immunol Rev 227:150-160.
Law, C. L., S. P. Sidorenko, K. A. Chandran, Z. Zhao, S. H. Shen, E. H. Fischer, and E. A. Clark. 1996. CD22 associates with protein tyrosine phosphatase 1C, Syk, and phospholipase C-gamma(1) upon B cell activation. J Exp Med 183:547-560.
Liu, Q. Y., R. R. Sooknanan, L. T. Malek, M. Ribecco-Lutkiewicz, J. X. Lei, H. Shen, B. Lach, P. R. Walker, J. Martin, and M. Sikorska. 2006. Novel subtractive transcription-based amplification of mRNA (STAR) method and its application in search of rare and differentially expressed genes in AD brains. BMC Genomics 7:286.
Mao, D., H. Epple, B. Uthgenannt, D. V. Novack, and R. Faccio. 2006. PLCgamma2 regulates osteoclastogenesis via its interaction with ITAM proteins and GAB2. J Clin Invest 116:2869-2879.
Muhlbauer, R. C., F. Bauss, R. Schenk, M. Janner, E. Bosies, K. Strein, and H. Fleisch. 1991. BM 21.0955, a potent new bisphosphonate to inhibit bone resorption. J Bone Miner Res 6:1003-1011.
O'Reilly, M. K., and J. C. Paulson. 2009. Siglecs as targets for therapy in immune-cell-mediated disease. Trends Pharmacol Sci 30:240-248.
Paloneva, J., M. Kestila, J. Wu, A. Salminen, T. Bohling, V. Ruotsalainen, P. Hakola, A. B. Bakker, J. H. Phillips, P. Pekkarinen, L. L. Lanier, T. Timonen, and L. Peltonen. 2000. Loss-of-function mutations in TYROBP (DAP12) result in a presenile dementia with bone cysts. Nat Genet 25:357-361.
Park-Wyllie, L. Y., M. M. Mamdani, D. N. Juurlink, G. A. Hawker, N. Gunraj, P. C. Austin, D. B. Whelan, P. J. Weiler, and A. Laupacis. 2011. Bisphosphonate use and the risk of subtrochanteric or femoral shaft fractures in older women. JAMA 305:783-789.
Raggatt, L. J., and N. C. Partridge. 2010. Cellular and molecular mechanisms of bone remodeling. J Biol Chem 285:25103-25108.
Reid, I. R., and J. Cornish. 2011. Epidemiology and pathogenesis of osteonecrosis of the jaw. Nat Rev Rheumatol 8:90-96.
Roelofs, A. J., K. Thompson, S. Gordon, and M. J. Rogers. 2006. Molecular mechanisms of action of bisphosphonates: current status. Clin Cancer Res 12:6222s-6230s.
Semler, O. et al., 2012. First use of the RANKL antibody denosumab in osteogenesis Imperfecta type VI. J. Musculoskelet Neuronal Interact 12(3):183-188.
Sooknanan, R. R., G. B. Tremblay, and M. Filion. 2011. Polynucleotides and polypeptide sequences involved in the process of bone remodeling. U.S. Pat. No. 7,989,160.
Stuible, M et al., 2014. MEchanism and function of monoclonal antibodies targeting siglec-15 for therapeutic inhibition of osteoclastic bone resorption. J. Biol. Chem 289(10):6498-6512.
Takahata, M., N. Iwasaki, H. Nakagawa, Y. Abe, T. Watanabe, M. Ito, T. Majima, and A. Minami. 2007. Sialylation of cell surface glycoconjugates is essential for osteoclastogenesis. Bone 41:77-86.
Taylor, V. C., C. D. Buckley, M. Douglas, A. J. Cody, D. L. Simmons, and S. D. Freeman. 1999. The myeloid-specific sialic acid-binding receptor, CD33, associates with the protein-tyrosine phosphatases, SHP-1 and SHP-2. J Biol Chem 274:11505-11512.
Tomasello, E., and E. Vivier. 2005. KARAP/DAP12/TYROBP: three names and a multiplicity of biological functions. Eur J Immunol 35:1670-1677.
Toyomura, T., Y. Murata, A. Yamamoto, T. Oka, G. H. Sun-Wada, Y. Wada, and M. Futai. 2003. From lysosomes to the plasma membrane: localization of vacuolar-type H+-ATPase with the a3 isoform during osteoclast differentiation. J Biol Chem 278:22023-22030.
Turnbull, I. R., and M. Colonna. 2007. Activating and inhibitory functions of DAP12. Nat Rev Immunol 7:155-161.
Underhill, D. M., and H. S. Goodridge. 2007. The many faces of ITAMs. Trends Immunol 28:66-73.
Walker, J. A., and K. G. Smith. 2008. CD22: an inhibitory enigma. Immunology 123:314-325.
Walsh, M. C., and Y. Choi. 2003. Biology of the TRANCE axis. Cytokine Growth Factor Rev 14:251-263.

### Sequences

Note: Underlined sequences represent the constant region, twice-underlined sequences represent the optional signal peptide and sequences in bold represent complementarity determining regions.

(human Siglec-15 cDNA)
SEQ ID NO: 1
ATGGAAAAGTCCATCTGGCTGCTGGCCTGCTTGGCGTGGGTTCTCCCGACAGGCTCATTTGTGAGAACT
AAAATAGATACTACGGAGAACTTGCTCAACACAGAGGTGCACAGCTCGCCAGCGCAGCGCTGGTCCATG
CAGGTGCCACCCGAGGTGAGCGCGGAGGCAGGCGACGCGGCAGTGCTGCCCTGCACCTTCACGCACCCG
CACCGCCACTACGACGGGCCGCTGACGGCCATCTGGCGCGCGGGCGAGCCCTATGCGGGCCCGCAGGTG
TTCCGCTGCGCTGCGGCGCGGGGCAGCGAGCTCTGCCAGACGGCGCTGAGCCTGCACGGCCGCTTCCGG
CTGCTGGGCAACCCGCGCCGCAACGACCTCTCGCTGCGCGTCGAGCGCCTCGCCCTGGCTGACGACCGC
CGCTACTTCTGCCGCGTCGAGTTCGCCGGCGACGTCCATGACCGCTACGAGAGCCGCCACGGCGTCCGG
CTGCACGTGACAGCCGCGCCGCGGATCGTCAACATCTCGGTGCTGCCCAGTCCGGCTCACGCCTTCCGC
GCGCTCTGCACTGCCGAAGGGGAGCCGCCGCCCGCCCTCGCCTGGTCCGGCCCGGCCCTGGGCAACAGC
TTGGCAGCCGTGCGGAGCCCGCGTGAGGGTCACGGCCACCTAGTGACCGCCGAACTGCCCGCACTGACC
CATGACGGCCGCTACACGTGTACGGCCGCCAACAGCCTGGGCCGCTCCGAGGCCAGCGTCTACCTGTTC
CGCTTCCATGGCGCCAGCGGGGCCTCGACGGTCGCCCTCCTGCTCGGCGCTCTCGGCTTCAAGGCGCTG
CTGCTGCTCGGGGTCCTGGCCGCCCGCGCTGCCCGCCGCCGCCCAGAGCATCTGGACACCCCGGACACC
CCACCACGGTCCCAGGCCCAGGAGTCCAATTATGAAAATTTGAGCCAGATGAACCCCCGGAGCCCACCA
GCCACCATGTGCTCACCGTGA
(Human Siglec-15 polypeptide: 1-328)
SEQ ID NO: 2
MEKSIWLLACLAWVLPTGSFVRTKIDTTENLLNTEVHSSPAQRWSMQVPPEVSAEAGDAAVLPCTFTHP
HRHYDGPLTAIWRAGEPYAGPQVFRCAAARGSELCQTALSLHGRFRLLGNPRRNDLSLRVERLALADDR
RYFCRVEFAGDVHDRYESRHGVRLHVTAAPRIVNISVLPSPAHAFRALCTAEGEPPPALAWSGPALGNS
LAAVRSPREGHGHLVTAELPALTHDGRYTCTAANSLGRSEASVYLFRFHGASGASTVALLLGALGFKAL
LLLGVLAARAARRRPEHLDTPDTPPRSQAQESNYENLSQMNPRSPPATMCSP
(mouse Siglec-15 cDNA)
SEQ ID NO: 3
ATGGAGGGGTCCCTCCAACTCCTGGCCTGCTTGGCCTGTGTGCTCCAGATGGGATCCCTTGTGAAAACT
AGAAGAGACGCTTCGGGGGATCTGCTCAACACAGAGGCGCACAGTGCCCCGGCGCAGCGCTGGTCCATG
CAGGTGCCCGCGGAGGTGAACGCGGAGGCTGGCGACGCGGCGGTGCTGCCCTGCACCTTCACGCACCCG
CACCGCCACTACGACGGGCCGCTGACGGCCATCTGGCGCTCGGGCGAGCCGTACGCGGGCCCGCAGGTG
TTCCGCTGCACCGCGGCGCCGGGCAGCGAGCTGTGCCAGACGGCGCTGAGCCTGCACGGCCGCTTCCGC
CTGCTGGGCAACCCGCGCCGCAACGACCTGTCCCTGCGCGTCGAGCGCCTCGCCCTGGCGGACAGCGGC
CGCTACTTCTGCCGCGTGGAGTTCACCGGCGACGCCCACGATCGCTATGAGAGTCGCCATGGGGTCCGT
CTGCGCGTGACTGCAGCTGCGCCGCGGATCGTCAACATCTCGGTGCTGCCGGGCCCCGCGCACGCCTTC
CGCGCGCTCTGCACCGCCGAGGGGGAGCCCCCGCCCGCCCTCGCCTGGTCGGGTCCCGCCCCAGGCAAC
AGCTCCGCTGCCCTGCAGGGCCAGGGTCACGGCTACCAGGTGACCGCCGAGTTGCCCGCGCTGACCCGC
GACGGCCGCTACACGTGCACGGCGGCCAATAGCCTGGGCCGCGCCGAGGCCAGCGTCTACCTGTTCCGC
TTCCACGGCGCCCCCGGAACCTCGACCCTAGCGCTCCTGCTGGGCGCGCTGGGCCTCAAGGCCTTGCTG
CTGCTTGGCATTCTGGGAGCGCGTGCCACCCGACGCCGACTAGATCACCTGGTCCCCCAGGACACCCCT
CCACGGTCTCAGGCTCAGGAGTCCAATTATGAAAATTTGAGCCAGATGAGTCCTCCAGGCCACCAGCTG
CCACGTGTTTGCTGTGAGGAACTCCTCAGCCATCACCATCTAGTCATTCACCATGAGAAATAA
(mouse Siglec-15 polypeptide)
SEQ ID NO: 4
MEGSLQLLACLACVLQMGSLVKTRRDASGDLLNTEAHSAPAQRWSMQVPAEVNAEAGDAAVLPCTFTHP
HRHYDGPLTAIWRSGEPYAGPQVFRCTAAPGSELCQTALSLHGRFRLLGNPRRNDLSLRVERLALADSG
RYFCRVEFTGDAHDRYESRHGVRLRVTAAAPRIVNISVLPGPAHAFRALCTAEGEPPPALAWSGPAPGN
SSAALQGQGHGYQVTAELPALTRDGRYTCTAANSLGRAEASVYLFRFHGAPGTSTLALLLGALGLKALL
LLGILGARATRRRLDHLVPQDTPPRSQAQESNYENLSQMSPPGHQLPRVCCEELLSHHHLVIHHEK
25E9 Light (Kappa) Chain Chimeric (mouse variable domain and human
constant region)
SEQ ID No.: 5
MVLQTQVFISLLLWISGAYGDIVMTQAAPSVPVTPGESVSISCRSTKSLLHSNGNTYLYWFLQRPGQSP
QLLIYRMSNLASGVPDRFSGSGSGTAFTLRISRVEAEDVGVYYCMQHLEYPFTFGGGTKLEIKRTVAAP
SVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
25E9 Light Chain mouse variable domain (illustrated without signal
peptide: CDRs are in bold)
SEQ ID No.: 6
DIVMTQAAPSVPVTPGESVSISCRSTKSLLHSNGNTYLYWFLQRPGQSPQLLIYRMSNLASGVPDRFSG
SGSGTAFTLRISRVEAEDVGVYYCMQHLEYPFTFGGGTKLEIK
25E9 Light (Kappa) Chain Humanized Variant 1 (a.k.a.: L1) (humanized
variable domain and human constant region)
SEQ ID NO.: 7
MVLQTQVFISLLLWISGAYGDIVMTQSPLSLPVTPGEPASISCRSTKSLLHSNGNTYLYWYLQKPGQSP
QLLIYRMSNLASGVPDRFSGSGSGTDFTLKISRVEAEDVGVYYCMQHLEYPFTFGGGTKVEIKRTVAAP
SVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
25E9 Light Chain Humanized Variant 1 variable domain (a.k.a.: VL1)
(illustrated without signal peptide)
SEQ ID NO.: 8
DIVMTQSPLSLPVTPGEPASISCRSTKSLLHSNGNTYLYWYLQKPGQSPQLLIYRMSNLASGVPDRFSG
SGSGTDFTLKISRVEAEDVGVYYCMQHLEYPFTFGGGTKVEIK
25E9 Light (Kappa) Chain Humanized Variant 2 (a.k.a.: L2) (humanized
variable domain and human constant region)
SEQ ID NO.: 9
MVLQTQVFISLLLWISGAYGDIVMTQSPLSLPVTPGEPASISCRSTKSLLHSNGNTYLYWFLQKPGQSP
QLLIYRMSNLASGVPDRFSGSGSGTDFTLKISRVEAEDVGVYYCMQHLEYPFTFGGGTKVEIKRTVAAP
SVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
25E9 Light Chain Humanized Variant 2 variable domain (a.k.a.: VL2)
(illustrated without signal peptide)
SEQ ID NO.: 10
DIVMTQSPLSLPVTPGEPASISCRSTKSLLHSNGNTYLYWFLQKPGQSPQLLIYRMSNLASGVPDRFSG
SGSGTDFTLKISRVEAEDVGVYYCMQHLEYPFTFGGGTKVEIK
25E9 Heavy (Igg1) Chain Chimeric (mouse variable domain and human
constant region)
SEQ ID NO.: 11
MDWTWRILFLVAAATGTHAEIQLQQSGVELVRPGASVTLSCKASGYTFTDYDMHWVKQTPVHGLEWIGT
IDPETGGTAYNQKFKGKATLTADRSSTTAYMELSSLTSEDSAVYYCTSFYYTYSNYDVGFAYWGQGTLV
TVSAASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGK
25E9 Heavy Chain mouse variable domain (illustrated without signal
peptide: CDRs are in bold)
SEQ ID NO.: 12
EIQLQQSGVELVRPGASVTLSCKASGYTFTDYDMHWVKQTPVHGLEWIGTIDPETGGTAYNQKFKGKATL
TADRSSTTAYMELSSLTSEDSAVYYCTSFYYTYSNYDVGFAYWGQGTLVTVSA
25E9 Heavy (Igg1) Chain Humanized Variant 1 (a.k.a.: H1) (humanized
variable domain and human constant region)
SEQ ID NO.: 13
MDWTWRILFLVAAATGTHAEIQLQQSGAEVKKPGSSVKVSCKASGYTFTDYDMHWVRQAPGQGLEWMGT
IDPETGGTAYNQKFKGRVTITADKSTSTAYMELSSLRSEDTAVYYCTSFYYTYSNYDVGFAYWGQGTLV
TVSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGK
25E9 Heavy Chain Humanized Variant 1 variable domain (a.k.a., VH1)
(illustrated without signal peptide)
SEQ ID NO.: 14
EIQLQQSGAEVKKPGSSVKVSCKASGYTFTDYDMHWVRQAPGQGLEWMGTIDPETGGTAYNQKFKGRVT
ITADKSTSTAYMELSSLRSEDTAVYYCTSFYYTYSNYDVGFAYWGQGTLVTVSS
25E9 Heavy (Igg1) Chain Humanized Variant 2 (a.k.a.: H2) (humanized
variable domain and human constant region)
SEQ ID NO.: 15
MDWTWRILFLVAAATGTHAEIQLQQSGAEVKKPGSSVKVSCKASGYTFTDYDMHWVRQAPGQGLEWIGT
IDPETGGTAYNQKFKGRATLTADRSTSTAYMELSSLRSEDTAVYYCTSFYYTYSNYDVGFAYWGQGTLV
TVSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGK
25E9 Heavy Chain Humanized Variant 2 variable domain (a.k.a., VH2)
(illustrated without signal peptide)
SEQ ID NO.: 16
EIQLQQSGAEVKKPGSSVKVSCKASGYTFTDYDMHWVRQAPGQGLEWIGTIDPETGGTAYNQKFKGRAT
LTADRSTSTAYMELSSLRSEDTAVYYCTSFYYTYSNYDVGFAYWGQGTLVTVSS
25E9 Heavy (Igg1) Chain Humanized Variant 3 (a.k.a.: H3) (humanized
variable domain and human constant region)
SEQ ID NO.: 17
MDWTWRILFLVAAATGTHAEIQLQQSGAEVKKPGSSVKVSCKASGYTFTDYDMHWVKQAPGQGLEWIGT
IDPETGGTAYNQKFKGKATLTADRSTSTAYMELSSLRSEDTAVYYCTSFYYTYSNYDVGFAYWGQGTLV
TVSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGK
25E9 Heavy Chain Humanized Variant 3 variable domain (a.k.a.: VH3)
(illustrated without signal peptide)
SEQ ID NO.: 18
EIQLQQSGAEVKKPGSSVKVSCKASGYTFTDYDMHWVKQAPGQGLEWIGTIDPETGGTAYNQKFKGKAT
LTADRSTSTAYMELSSLRSEDTAVYYCTSFYYTYSNYDVGFAYWGQGTLVTVSS
25E9 Heavy (Igg1) Chain Humanized Variant 4 (a.k.a.: H4) (humanized
variable domain and human constant region)
SEQ ID NO.: 19
MDWTWRILFLVAAATGTHAEIQLQQSGAEVKKPGSSVKVSCKASGYTFTDYDMHWVKQAPGHGLEWIGT
IDPETGGTAYNQKFKGKATLTADRSTSTAYMELSSLTSEDTAVYYCTSFYYTYSNYDVGFAYWGQGTLV
TVSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGK
25E9 Heavy Chain Humanized Variant 4 variable domain (a.k.a.: VH4)
(illustrated without signal peptide)
SEQ ID NO.: 20
EIQLQQSGAEVKKPGSSVKVSCKASGYTFTDYDMHWVKQAPGHGLEWIGTIDPETGGTAYNQKFKGKAT
LTADRSTSTAYMELSSLTSEDTAVYYCTSFYYTYSNYDVGFAYWGQGTLVTVSS
Chimeric 25D8 Light (Kappa) Chain (mouse variable domain and human
constant region)
SEQ ID NO.: 21
MVLQTQVFISLLLWISGAYGDIVMTQAAFSNPVTLGTSASISCRSSKSLLHSNGITYLYWYLQKPGQSP
QLLIYQMSNLASGVPDRFSSSGSGTDFTLRISRVEAEDVGVYYCAQNLELPYTFGGGTKLEIKRTVAAP
SVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
25D8 Light Chainmouse variable domain (illustrated without signal
peptide)
SEQ ID NO.: 22
DIVMTQAAFSNPVTLGTSASISCRSSKSLLHSNGITYLYWYLQKPGQSPQLLIYQMSNLASGVPDRFSS
SGSGTDFTLRISRVEAEDVGVYYCAQNLELPYTFGGGTKLEIK
Humanized 25D8 Light (Kappa) Chain (humanized variable domain and
human constant region)
SEQ ID NO.: 23
MVLQTQVFISLLLWISGAYGDIVMTQTPLSLPVTPGEPASISCRSSKSLLHSNGITYLYWYLQKPGQSP
QLLIYQMSNLASGVPDRFSGSGSGTDFTLKISRVEAEDVGVYYCAQNLELPYTFGGGTKVEIKRTVAAP
SVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
Humanized 25D8 Light Chain variable domain (illustrated without
signal peptide)
SEQ ID NO.: 24
DIVMTQTPLSLPVTPGEPASISCRSSKSLLHSNGITYLYWYLQKPGQSPQLLIYQMSNLASGVPDRFSG
SGSGTDFTLKISRVEAEDVGVYYCAQNLELPYTFGGGTKVEIK
Chimeric 25D8 Heavy (Igg2) Chain (mouse variable domain and human
constant region)
SEQ ID NO.: 25
MDWTWRILFLVAAATGTHAQVQVQQPGAELVKPGASVKLSCKASGYTFTSYWMHWVKQRPGQGLEWIGL
INPSNARTNYNEKFNTKATLTVDKSSSTAYMQLSSLTSEDSAVYYCARGGDGDYFDYWGQGTTLTVSSA
STKGPSVFPLAPCSRSTSESTAALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPSVFLFPPKPKDTLMISRTPEVT
CVVVDVSHEDPEVQFNWYVDGVEVHNAKTKPREEQFNSTFRVVSVLTVVHQDWLNGKEYKCKVSNKGLP
APIEKTISKTKGQPREPQVYTLPPSREEMTKNQVSLTCLVKGFYPSDIAVEWESNGQPENNYKTTPPML
DSDGSFFLYSKLTVDKSRWQQGNVFSCSVMHEALHNHYTQKSLSLSPGK
25D8 Heavy Chain mouse variable domain (illustrated without signal
peptide)
SEQ ID NO.: 26
QVQVQQPGAELVKPGASVKLSCKASGYTFTSYWMHWVKQRPGQGLEWIGLINPSNARTNYNEKFNTKAT
LTVDKSSSTAYMQLSSLTSEDSAVYYCARGGDGDYFDYWGQGTTLTVSS
Humanized 25D8 Heavy (Igg2) Chain (humanized variable domain and
human constant region)
SEQ ID NO.: 27
MDWTWRILFLVAAATGTHAQVQLQQSGAEVKKPGSSVKVSCKASGYTFTSYWMHWVRQAPGQGLEWMGL
INPSNARTNYNEKFNTRVTITADKSTSTAYMELSSLRSEDTAVYYCARGGDGDYFDYWGQGTTVTVSSA
STKGPSVFPLAPCSRSTSESTAALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPSVFLFPPKPKDTLMISRTPEVT
CVVVDVSHEDPEVQFNWYVDGVEVHNAKTKPREEQFNSTFRVVSVLTVVHQDWLNGKEYTCKVSNKGLP
APIEKTISKTKGQPREPQVYTLPPSREEMTKNQVSLTCLVKGFYPSDIAVEWESNGQPENNYKTTPPML
DSDGSFFLYSKLTVDKSRWQQGNVFSCSVMHEALHNHYTQKSLSLSPGK
Humanized 25D8 Heavy Chain variable domain (illustrated without
signal peptide)
SEQ ID NO.: 28
QVQLQQSGAEVKKPGSSVKVSCKASGYTFTSYWMHWVRQAPGQGLEWMGLINPSNARTNYNEKFNTRVT
ITADKSTSTAYMELSSLRSEDTAVYYCARGGDGDYFDYWGQGTTVTVSS
25E9 Heavy (Igg2) Chain Humanized Variant 1 (humanized variable
domain and human constant region)
SEQ ID NO. 29
MDWTWRILFLVAAATGTHAEIQLQQSGAEVKKPGSSVKVSCKASGYTFTDYDMHWVRQAPGQGLEWMGT
IDPETGGTAYNQKFKGRVTITADKSTSTAYMELSSLRSEDTAVYYCTSFYYTYSNYDVGFAYWGQGTLV
TVSSASTKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAGPSVFLFPPKPKDTLMISR
TPEVTCVVVDVSHEDPEVQFNWYVDGVEVHNAKTKPREEQFNSTFRVVSVLTVVHQDWLNGKEYKCKVS
NKGLPAPIEKTISKTKGQPREPQVYTLPPSREEMTKNQVSLTCLVKGFYPSDIAVEWESNGQPENNYKT
TPPMLDSDGSFFLYSKLTVDKSRWQQGNVFSCSVMHEALHNHYTQKSLSLSPGK
25E9 Heavy (Igg2) Chain Chimeric (mouse variable domain and human
constant region)
SEQ ID NO.: 30
MDWTWRILFLVAAATGTHAEIQLQQSGVELVRPGASVTLSCKASGYTFTDYDMHWVKQTPVHGLEWIGT
IDPETGGTAYNQKFKGKATLTADRSSTTAYMELSSLTSEDSAVYYCTSFYYTYSNYDVGFAYWGQGTLV
TVSAASTKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAGPSVFLFPPKPKDTLMISR
TPEVTCVVVDVSHEDPEVQFNWYVDGVEVHNAKTKPREEQFNSTFRVVSVLTVVHQDWLNGKEYKCKVS
NKGLPAPIEKTISKTKGQPREPQVYTLPPSREEMTKNQVSLTCLVKGFYPSDIAVEWESNGQPENNYKT
TPPMLDSDGSFFLYSKLTVDKSRWQQGNVFSCSVMHEALHNHYTQKSLSLSPGK
(human IgG1 constant region)
SEQ ID NO.:31
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGK
(human IgG2 constant region)
SEQ ID NO.:32
ASTKGPSVFPLAPCSRSTSESTAALGCLVKDYFPEPVTVSWNSGALTSGVHTFPAVLQSSGLYSLSSVV
TVPSSNFGTQTYTCNVDHKPSNTKVDKTVERKCCVECPPCPAPPVAGPSVFLFPPKPKDTLMISRTPEV
TCVVVDVSHEDPEVQFNWYVDGVEVHNAKTKPREEQFNSTFRVVSVLTVVHQDWLNGKEYKCKVSNKGL
PAPIEKTISKTKGQPREPQVYTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPGK
Generic 25E9 light chain variable domain (consensus 1)
SEQ ID NO. 33
DIVMTQXXXSXPVTPGEXXSISCRSTKSLLHSNGNTYLYWXLQXPGQSPQLLIYRMSNLASGVPDRFSG
SGSGTXFTLXISRVEAEDVGVYYCMQHLEYPFTFGGGTKXEIK

wherein at least one of the amino acid identified by X may be an amino acid substitution in comparison with a corresponding amino acid in the polypeptide set forth in SEQ ID NO.:6 (the mouse VL). The amino acid substitution may be, for example conservative or non-conservative. In accordance with the invention, the amino acid substitution may be conservative.

Generic 25E9 light chain variable domain
(consensus 2)
SEQ ID NO. 34
DIVMTQXₐ₁Xₐ₂Xₐ₃SXₐ₄PVTPGEXₐ₅Xₐ₆SISCRSTKSLLHSNGNTYLYW
Xₐ₇LQXₐ₈PGQSPQLLIYRMSNLASGVPDRFSGSGSGTXₐ₉FTLXₐ₁₀ISR
VEAEDVGVYYCMQHLEYPFTFGGGTKXₐ₁₁EIK

wherein at least one of the amino acid identified by X may be an amino acid substitution in comparison with a corresponding amino acid in the polypeptide set forth in SEQ ID NO.:6 (the mouse VL) and Wherein Xa1, Xa4, Xa7, Xa8, Xa10 and Xa11 may each independently be a conservative amino acid substitution in comparison with SEQIDNO.6;
Wherein Xa2, Xa5, Xa6 may each independently be a semi-conservative amino acid substitution in comparison with SEQIDNO.6;

### Wherein Xa3 may be P or L; and

### Wherein Xa9 may be A or D.

Generic 25E9 light chain variable domain
(consensus 3)
SEQ ID NO. 35
DIVMTQXₐ₁Xₐ₂Xₐ₃SXₐ₄PVTPGEXₐ₅Xₐ₆SISCRSTKSLLHSNGNTYLYW
Xₐ₇LQXₐ₈PGQSPQLLIYRMSNLASGVPDRFSGSGSGTXₐ₉FTLXₐ₁₀ISR
VEAEDVGVYYCMQHLEYPFTFGGGTKXₐ₁₁EIK

wherein at least one of the amino acid identified by X (including Xa1 to Xa11) may be an amino acid substitution in comparison with a corresponding amino acid in the polypeptide set forth in SEQ ID NO.:6 (the mouse VL) and

### Wherein Xa1 may be A or S;

### Wherein Xa2 may be A or P;

### Wherein Xa3 may be P or L;

Wherein Xa4 may be a hydrophobic amino acid (e.g., V or L);

### Wherein Xa5 may be S or P;

Wherein Xa6 may be a hydrophobic amino acid (e.g., V or A);
Wherein Xa7 may be an aromatic amino acid (e.g. F or Y);
Wherein Xa8 may be a basic amino acid (e.g., R or K);

### Wherein Xa9 may be A or D;

Wherein Xa10 may be a basic amino acid (e.g., R or K);
and wherein Xa11 may be a hydrophobic amino acid (e.g., L or V).

Generic 25E9 heavy chain variable domain
(consensus 1)
SEQ ID NO. 36
EIQLQQSGXEXXXPGXSVXXSCKASGYTFTDYDMHWVXQXPXXGLEWXG
TIDPETGGTAYNQKFKGXXTXTADXSXXTAYMELSSLXSEDXAVYYCTS
FYYTYSNYDVGFAYWGQGTLVTVSX

wherein at least one of the amino acid identified by X may be an amino acid substitution in comparison with a corresponding amino acid in the polypeptide set forth in SEQ ID NO.:12 (the mouse VH). The amino acid substitution may be, for example conservative or non-conservative. In accordance with the invention, the amino acid substitution may be conservative.

Generic 25E9 heavy chain variable domain
(consensus 2)
SEQ ID NO. 37
EIQLQQSGX_{b1}EX_{b2}X_{b3}X_{b4}PGX_{b5}SVX_{b6}X_{b7}SCKASGYTFTDYDMHWVX_{b8}Q
X_{b9}P_{Xb10}X_{b11}GLEWX_{b12}GTIDPETGGTAYNQKFKGX_{b13}X_{b14}TX_{b15}TADX_{b16}
SX_{b17}X_{b18}TAYMELSSLX_{b19}SEDX_{b20}AVYYCTSFYYTYSNYDVGFAYWGQG
TLVTVSX_{b21}

wherein at least one of the amino acid identified by X (including Xb1 to Xb21) may be an amino acid substitution in comparison with a corresponding amino acid in the polypeptide set forth in SEQ ID NO.:12 (the mouse VH) and
wherein Xb2, Xb4, Xb5, Xb7, Xb8, Xb9, Xb11, Xb12, Xb13, Xb15, Xb16, Xb17, Xb18, Xb20 and Xb21 may each independently be a conservative amino acid substitution in comparison with SEQIDNO.12;
wherein Xb1, Xb6, Xb14 may each independently be a semi-conserved amino acid substitution in comparison with SEQIDNO.:12 (the mouse VH)
wherein Xb3 may be V or K;
wherein Xb10 may be V or G; and
wherein Xb19 may be T or R.

Generic 25E9 heavy chain variable domain
(consensus 3)
SEQ ID NO. 38
EIQLQQSGX_{b1}EX_{b2}X_{b3}X_{b4}PGX_{b5}SVX_{b6}X_{b7}SCKASGYTFTDYDMHWV
X_{b8}QX_{b9}P_{Xb10}X_{b11}GLEWX_{b12}GTIDPETGGTAYNQKFKGX_{b13}X_{b14}T
X_{b15}TADX_{b16}SX_{b17}X_{b18}TAYMELSSLX_{b19}SEDX_{b20}AVYYCTSFYYTY
SNYDVGFAYWGQGTLVTVSX_{b21}

wherein at least one of the amino acid identified by X (including Xb1 to Xb21) may be an amino acid substitution in comparison with a corresponding amino acid in the polypeptide set forth in SEQ ID NO.:12 (the mouse VH) and
wherein Xb1 may be a hydrophobic amino acid (e.g., V or A);
wherein Xb2 may be a hydrophobic amino acid (e.g., L or V);
wherein Xb3 may be V or K;
wherein Xb4 may be a basic amino acid (e.g., R or K);

### Wherein Xb5 may be A or S;

### Wherein Xb6 may be T or K;

Wherein Xb7 may be a hydrophobic amino acid (e.g., L or V);
Wherein Xb8 may be a basic amino acid (e.g., K or R);

### Wherein Xb9 may be T or A;

wherein Xb10 may be V or G;
Wherein Xb11 may be a basic amino acid (e.g., H or Q);
Wherein Xb12 may be a hydrophobic amino acid (e.g., I or M);
Wherein Xb13 may be a basic amino acid (e.g., K or R);
Wherein Xb14 may be a hydrophobic amino acid (e.g., A or V);
Wherein Xb15 may be a hydrophobic amino acid (e.g., L or I);
Wherein Xb16 may be a basic amino acid (e.g., R or K);
Wherein Xb17 may be a neutral hydrophilic amino acid (e.g., S or T);
Wherein Xb18 may be a neutral hydrophilic amino acid (e.g., T or S);
wherein Xb19 may be T or R;
Wherein Xb20 may be a neutral hydrophilic amino acid (e.g., S or T); and

### Wherein Xb21 may be A or S.

Generic 25D8 light chain variable domain
(consensus 1)
SEQ ID No.: 39
DIVMTQXXXSXPVTXGXXASISCRSSKSLLHSNGITYLYWYLQKPGQSP
QLLIYQMSNLASGVPDRFSXSGSGTDFTLXISRVEAEDVGVYYCAQNLE
LPYTFGGGTKXEIK

wherein at least one of the amino acid identified by X may be an amino acid substitution in comparison with a corresponding amino acid in the polypeptide set forth in SEQ ID NO.:22 (the mouse VL). The amino acid substitution may be, for example conservative or non-conservative. In accordance with the invention, the amino acid substitution may be conservative.

Generic 25D8 light chain variable domain
(consensus 2)
SEQ ID NO.: 40
DIVMTQX_{c1}X_{c2}X_{c3}SX_{c4}PVTX_{c5}GX_{c6}X_{c7}ASISCRSSKSLLHSNGITYLY
WYLQKPGQSPQLLIYQMSNLASGVPDRFSX_{c8}SGSGTDFTLX_{c9}ISRVE
AEDVGVYYCAQNLELPYTFGGGTKX_{c10}EIK

wherein at least one of the amino acid identified by X may be an amino acid substitution in comparison with a corresponding amino acid in the polypeptide set forth in SEQ ID NO.:22 (the mouse VL) and
wherein Xc1, Xc3, Xc9 and Xc10 may each independently be a conservative amino acid substitution in comparison with SEQ ID NO.:22;
wherein Xc2, Xc7, Xc8 may each independently be a semi-conservative amino acid substitution in comparison with SEQ ID NO.: 22;

### Wherein Xc4 may be N or L;

### Wherein Xc5 may be L or P; and

### Wherein Xc6 may be T or E.

Generic 25D8 light chain variable domain
(consensus 3)
SEQ ID NO.: 41
DIVMTQX_{c1}X_{c2}X_{c3}SX_{c4}PVTX_{c5}GX_{c6}X_{c7}ASISCRSSKSLLHSNGITYLY
WYLQKPGQSPQLLIYQMSNLASGVPDRFSX_{c8}SGSGTDFTLX_{c9}ISRVE
AEDVGVYYCAQNLELPYTFGGGTKX_{c10}EIK

wherein at least one of the amino acid identified by X may be an amino acid substitution in comparison with a corresponding amino acid in the polypeptide set forth in SEQ ID NO.:22 (the mouse VL) and

### Wherein Xc1 may be A or T;

### Wherein Xc2 may be A or P;

### Wherein Xc3 may be F or L;

### Wherein Xc4 may be N or L;

### Wherein Xc5 may be L or P;

### Wherein Xc6 may be T or E;

### Wherein Xc7 may be S or P;

### Wherein Xc8 may be S or G;

Wherein Xc9 may be a basic amino acid (e.g., R or K); and
Wherein Xc10 may be a hydrophobic amino acid (e.g., L or V).

Generic 25D8 heavy chain variable domain
(consensus 1)
SEQ ID NO.: 42
QVQXQQXGAEXXKPGXSVKXSCKASGYTFTSYWMHWVXQXPGQGLEWXG
LINPSNARTNYNEKFNTXXTXTXDKSXSTAYMXLSSLXSEDXAVYYCAR
GGDGDYFDYWGQGTTXTVSS

wherein at least one of the amino acid identified by X may be an amino acid substitution in comparison with a corresponding amino acid in the polypeptide set forth in SEQ ID NO.:26 (the mouse VH). The amino acid substitution may be, for example conservative or non-conservative. In accordance with the invention, the amino acid substitution may be conservative.

Generic 25D8 heavy chain variable domain
(consensus 2)
SEQ ID NO.: 43
QVQX_{d1}QQX_{d2}GAEX_{d3}X_{d4}KPGX_{d5}SVKX_{d6}SCKASGYTFTSYWMHWVX_{d7}Q
X_{d8}PGQGLEWX_{d9}GLINPSNARTNYNEKFNTX_{d10}X_{d11}TX_{d12}Tx_{d13}DKS
X_{d14}STAYMX_{d15}LSSLX_{d16}SEDX_{d17}AVYYCARGGDGDYFDYWGQGTT
X_{d18}TVSS

wherein at least one of the amino acid identified by X may be an amino acid substitution in comparison with a corresponding amino acid in the polypeptide set forth in SEQ ID NO.:26 (the mouse VH) and;
wherein Xd1, Xd3, Xd5, Xd6, Xd7, Xd9, Xd10, Xd12, Xd14, Xd15, Xd17, Xd18 may each independently be a conservative amino acid substitution in comparison with SEQ ID NO.:26;
wherein Xd2, Xd11, Xd13, may each independently be a semi-conservative amino acid substitution in comparison with SEQ ID NO.:26;
wherein Xd4 may be V or K;
wherein Xd8 may be R or A; and;
wherein Xd16 may be T or R.

Generic 25D8 heavy chain variable domain
(consensus 3)
SEQ ID NO.: 44
QVQX_{d1}QQX_{d2}GAEX_{d3}X_{d4}KPGX_{d5}SVKX_{d6}SCKASGYTFTSYWMHWVX_{d7}Q
X_{d8}PGQGLEWX_{d9}GLINPSNARTNYNEKFNTX_{d10}X_{d11}TX_{d12}TX_{d13}DKS
X_{d14}STAYMX_{d15}LSSLX_{d16}SEDX_{d17}AVYYCARGGDGDYFDYWGQGTT
X_{d18}TVSS

wherein at least one of the amino acid identified by X may be an amino acid substitution in comparison with a corresponding amino acid in the polypeptide set forth in SEQ ID NO.:26 (the mouse VH) and;
wherein Xd1 may be a hydrophobic amino acid (e.g., V or L);
wherein Xd2 may be P or S;
wherein Xd3 may be a hydrophobic amino acid (e.g., L or V);
wherein Xd4 may be V or K;
wherein Xd5 may be A or S;
wherein Xd6 may be a hydrophobic amino acid (e.g., L or V);
wherein Xd7 may be a basic amino acid (e.g., K or R);
wherein Xd8 may be R or A;
wherein Xd9 may be a hydrophobic amino acid (e.g., I or M);
wherein Xd10 may be a basic amino acid (e.g., K or R);
wherein Xd11 may be a hydrophobic amino acid (e.g., A or V);
wherein Xd12 may be a hydrophobic amino acid (e.g., L or I);
wherein Xd13 may be a hydrophobic amino acid (V or A);
wherein Xd14 may be a neutral hydrophilic amino acid (e.g., S or T);
wherein Xd15 may be Q or E;
wherein Xd16 may be T or R.
wherein Xd17 may be a neutral hydrophilic amino acid (e.g., S or T); and
wherein Xd18 may be a hydrophobic amino acid (L or V).

SEQ ID NO.: 45 Chimeric 25D8 Heavy (Igg1) Chain (mouse variable domain
and human constant region)
MDWTWRILFLVAAATGTHAQVQVQQPGAELVKPGASVKLSCKASGYTFTSYWMHWVKQRPGQGLEWIGL
INPSNARTNYNEKFNTKATLTVDKSSSTAYMQLSSLTSEDSAVYYCARGGDGDYFDYWGQGTTLTVSSA
STKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO.: 46 Humanized 25D8 Heavy (Igg1) Chain (humanized variable
domain and human constant region)
MDWTWRILFLVAAATGTHAQVQLQQSGAEVKKPGSSVKVSCKASGYTFTSYWMHWVRQAPGQGLEWMGL
INPSNARTNYNEKFNTRVTITADKSTSTAYMELSSLRSEDTAVYYCARGGDGDYFDYWGQGTTVTVSSA
STKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGEYPSDIAVEWESNGQPENNYKTT
PPVLDSDGSFFLYSKLTVDKSRWQQGNVFSCSVMHEALHNHYTQKSLSLSPGK
SEQ ID NO.: 47 CDR1 of the 25E9 light chain mouse variable domain
RSTKSLLHSNGNTYLY
SEQ ID NO.: 48 CDR2 of the 25E9 light chain mouse variable domain
RMSNLAS
SEQ ID NO.: 49 CDR3 of the 25E9 light chain mouse variable domain
MQHLEYPFT
SEQ ID NO.: 50 CDR1 of the 25E9 heavy chain mouse variable domain
GYTFTDYDMH
SEQ ID NO.: 51 CDR2 of the 25E9 heavy chain mouse variable domain
TIDPETGGTAYNQKFKG
SEQ ID NO.: 52 CDR3 of the 25E9 heavy chain mouse variable domain
FYYTYSNYDVGFAY
SEQ ID NO.: 53 CDR1 of the 25D8 light chain mouse variable domain
RSSKSLLHSNGITYLY
SEQ ID NO.: 54 CDR2 of the 25D8 light chain mouse variable domain
QMSNLASG
SEQ ID NO.: 55 CDR3 of the 25D8 light chain mouse variable domain
AQNLELPYT
SEQ ID NO.: 56 CDR1 of the 25D8 heavy chain mouse variable domain
GYTFTSYWMH
SEQ ID NO.: 57 CDR2 of the 25D8 heavy chain mouse variable domain
LINPSNARTNYNEKFNT
SEQ ID NO.: 58 CDR3 of the 25D8 heavy chain mouse variable domain
GGDGDYFDY
SEQ ID NO.: 59 25E9 Heavy (Igg2) Chain Humanized Variant 2 (a.k.a.: H2)
(humanized variable domain and human constant region)
MDWTWRILFLVAAATGTHAEIQLQQSGAEVKKPGSSVKVSCKASGYTFTDYDMHWVRQAPGQGLEWIGT
IDPETGGTAYNQKFKGRATLTADRSTSTAYMELSSLRSEDTAVYYCTSFYYTYSNYDVGFAYWGQGTLV
TVSSASTKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAGPSVFLFPPKPKDTLMISR
TPEVTCVVVDVSHEDPEVQFNWYVDGVEVHNAKTKPREEQFNSTFRVVSVLTVVHQDWLNGKEYKCKVS
NKGLPAPIEKTISKTKGQPREPQVYTLPPSREEMTKNQVSLTCLVKGFYPSDIAVEWESNGQPENNYKT
TPPMLDSDGSFFLYSKLTVDKSRWQQGNVFSCSVMHEALHNHYTQKSLSLSPGK
SEQ ID NO.: 60 25E9 Heavy (Igg2) Chain Humanized Variant 3 (a.k.a.: H3)
(humanized variable domain and human constant region)
MDWTWRILFLVAAATGTHAEIQLQQSGAEVKKPGSSVKVSCKASGYTFTDYDMHWVKQAPGQGLEWIGT
IDPETGGTAYNQKFKGKATLTADRSTSTAYMELSSLRSEDTAVYYCTSFYYTYSNYDVGFAYWGQGTLV
TVSSASTKGPSVFPLAPCSRSTSESTAALGCLVKDYFPEPVTVSWNSGALTSGVHTFPAVLQSSGLYSL
SSVVTVPSSNEGTQTYTCNVDHKPSNTKVDKTVERKCCVECPPCPAPPVAGPSVFLEPPKPKDTLMISR
TPEVTCVVVDVSHEDPEVQFNWYVDGVEVHNAKTKPREEQFNSTERVVSVLTVVHQDWLNGKEYKCKVS
NKGLPAPIEKTISKTKGQPREPQVYTLPPSREEMTKNQVSLTCLVKGFYPSDIAVEWESNGQPENNYKT
TPPMLDSDGSFFLYSKLTVDKSRWQQGNVFSCSVMHEALHNHYTQKSLSLSPGK
SEQ ID NO.: 61 25E9 Heavy (Igg2) Chain Humanized Variant 4 (a.k.a.: H4)
(humanized variable domain and human constant region)
MDWTWRILFLVAAATGTHAEIQLQQSGAEVKKPGSSVKVSCKASGYTFTDYDMHWVKQAPGHGLEWIGT
IDPETGGTAYNQKFKGKATLTADRSTSTAYMELSSLTSEDTAVYYCTSFYYTYSNYDVGFAYWGQGTLV
TVSSASTKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAGPSVFLFPPKPKDTLMISR
TPEVTCVVVDVSHEDPEVQFNWYVDGVEVHNAKTKPREEQFNSTFRVVSVLTVVHQDWLNGKEYKCKVS
NKGLPAPIEKTISKTKGQPREPQVYTLPPSREEMTKNQVSLTCLVKGFYPSDIAVEWESNGQPENNYKT
TPPMLDSDGSFFLYSKLTVDKSRWQQGNVFSCSVMHEALHNHYTQKSLSLSPGK
SEQ ID No.: 62 (nucleotide sequence of the 25E9 Light Chain mouse
variable domain)
GATATTGTGATGACCCAGGCTGCACCCTCTGTACCTGTCACTCCTGGAGAGTCAGTATCCATCTCCTGC
AGGTCTACTAAGAGTCTCCTGCATAGTAATGGCAACACTTACTTGTATTGGTTCCTGCAGAGGCCAGGC
CAGTCTCCTCAGCTCCTGATATATCGGATGTCCAACCTTGCCTCAGGAGTCCCAGACAGGTTCAGTGGC
AGTGGGTCAGGAACTGCTTTCACACTGAGAATCAGTAGAGTGGAGGCTGAGGATGTGGGTGTTTATTAC
TGTATGCAACATCTAGAATATCCTTTCACGTTCGGAGGGGGGACCAAGCTGGAAATAAAA
SEQ ID NO: 63 (nucleotide sequence of the 25E9 heavy Chain mouse
variable domain)
GAGATCCAGCTGCAGCAGTCTGGAGTTGAGCTGGTGAGGCCTGGGGCTTCAGTGACGCTGTCCTGCAAG
GCTTCGGGCTACACATTTACTGACTATGACATGCACTGGGTGAAGCAGACACCTGTTCATGGCCTGGAA
TGGATTGGAACTATTGATCCTGAAACTGGTGGTACTGCCTACAATCAGAAGTTCAAGGGCAAGGCCACA
CTGACTGCGGACAGATCCTCCACCACAGCCTACATGGAGCTCAGCAGCCTGACATCTGAGGACTCTGCC
GTCTATTACTGTACAAGTTTCTACTATACTTACTCTAATTACGACGTGGGGTTTGCTTACTGGGGCCAA
GGGACTCTGGTCACTGTCTCTGCA
SEQ ID NO.: 64 (nucleotide sequence of the humanized 25E9 light chain
variable domain-variant 1-illustrated without the portion coding for
the signal peptide)
gacatcgtgatgacccagtcccccctgtccctgcctgtgacacctggcgagcccgcctccatctcctgc
cggtccaccaagtccctgctgcactccaacggcaacacctacctgtactggtatctgcagaagcccggc
cagtcccctcagctgctgatctaccggatgtccaacctggcctccggcgtgcccgacagattctccggc
tctggctccggcaccgacttcaccctgaagatctcccgggtggaagccgaggacgtgggcgtgtactac
tgcatgcagcacctggaataccccttcaccttcggcggaggcaccaaggtggaaatcaag
SEQ ID NO.: 65 (nucleotide sequence of the humanized 25E9 heavy chain
variable domain-variant 1-illustrated without the portion coding
for the signal peptide)
gagattcagctgcagcagtcaggagccgaagtgaagaaacccggctccagcgtcaaggtgagttgcaag
gcctccggatacactttcaccgactatgatatgcactgggtgagacaggcacctgggcagggtctggag
tggatggggaccatcgatccagaaaccggcggaacagcctacaaccagaagtttaaaggtcgagtgaca
attactgctgacaagtccaccagcacagcatatatggagctgtctagtctgcgttctgaagatacagcc
gtctactattgcacttctttctactacacctacagtaactacgacgtggggtttgcttactggggccag
ggaactctggtcaccgtgtcatcc
SEQ ID NO.: 66: candidate human model for 25D8 light chain variable
domain
DIVMTQTPLSLPVTPGEPASISCRSSQSLLHSNGYNYLDWYLQKPGQSPQLLIYLGSNRASGVPDRFSG
SGSGTDFTLKISRVEAEDVGVYYCMQGLQTPLTFGGGTKVEIK
SEQ ID NO.: 67: candidate human model for 25D8 light chain variable
domain
DIVMTQTPLSLPVTPGEPASISCRASQSLLHSEGYNYLDWYLQKPGQSPQLLIYLGSNRASGVPDRFSG
SGSGTDFTLRISRVEAEDVGVYYCMQGLQTPLTFGGGTKVEIK
SEQ ID NO.: 68: candidate human model for 25D8 light chain variable
domain
DIVMTQSPLSLSVTPGQPASISCKSSQSLLHSDGKTYLYWYLQKPGQPPQLLIYEVSNRFSGVPDRFSG
SGSGTDFTLKISRVEAEDVGVYYCMQSIQLPYTFGQGTKLEIK
SEQ ID NO.: 69: human model for 25D8 light chain variable domain
DIVMTQSPLSLPVTPGEPASISCRSSQSLLHSNGYNYLDWYLQKPGQSPQLLIYLGSNRASGVPDRFSG
SGSGTDFTLKISRVEAEDVGVYYCMQALQ
SEQ ID NO.: 70: candidate human model for 25D8 light chain variable
domain
DIVMTQPPLSLPVTPGEPASISCRSSQSLLHSNGYNYLDWYLQKPGQSPQLLIYLGSNRASGVPDRFSG
SGSGTDFTLKISRVEAEDVGVYYCMQALQ
SEQ ID NO.: 71: candidate human model for 25D8 light chain variable
domain
DIVMTQTPLSLSVTPGQPASISCKSSQSLLHSDGKTYLYWYLQKPGQSPQLLIYEVSNRFSGVPDRFSG
SGSGTDFTLKISRVEAEDVGVYYCMQSIQL
SEQ ID NO.: 72: candidate human model for 25D8 light chain variable
domain
DIVMTQSPLSLPVTPGEPASISCRSSQSLLHSDGNNYLNWYLQKPGQSPQLLIYLVSNRASGVPDRFSG
SGSGTDFTLKISRVEAEDVGVYYCMQALQPRXTFGQGTKVEIK
SEQ ID NO.: 73: candidate human model for 25D8 heavy chain variable
domain
QVQLQQSGAEVKKPGSSVKVSCKASGGTFGSYAISWVRQAPGQGLEWMGRIIPILGIATYAQKFQGRVT
ITADKSTSTAYMDLSSLRSEDTAVYYCARGKGEFEGMDVWGQGTTVTVSS
SEQ ID NO.: 74: candidate human model for 25D8 heavy chain variable
domain
QVQLQQSGAEVKKPGSSVKVSCKASGGTFSSYAISWVRQAPGQGLEWMGRIIPILGIANYAQKFQGRVT
ITADKSTSTAYMELSSLRSEDTAVYYCARDTHSWFAFDIWGQGTMVTVSS
SEQ ID NO.: 75: candidate human model for 25D8 heavy chain variable
domain
EVQLVQSGAEMKKPGASVKVSCKASGYSFSIYNIHWVRQAPGQGLEWMGWIHAGTGNRKYSQVFQDRVT
ITRDTSASTSYMELSSLTSEDTAVYYCARDPNFGDFDSWGQGTLVTVSS
SEQ ID NO.: 76: candidate human model for 25D8 heavy chain variable
domain
QVQLVQSGAEVKKPGSSVKVSCKASGGTFSSYAISWVRQAPGQGLEWMGGIIPIFGTANYAQKFQGRVT
ITADESTSTAYMELSSLRSEDTAVYYCARMYNWNFFDYWGQGTLVTVSS
SEQ ID NO.: 77: candidate human model for 25D8 heavy chain variable
domain
QVQLVQSGAEVKKPGASVKVSCKASGYTFTSYYMHWVRQAPGQGLEWMGIINPSGGSTSYAQKFQGRVT
MTRDTSTSTVYMELSSLRSEDTAVYYCAREGDGYIQAFDYWGQGTLVTVSS
SEQ ID NO.: 78: candidate human model for 25D8 heavy chain variable
domain
QVQLVQSGAEVKKPGSSVKVSCKASGGTFSSYAISWVRQAPGQGLEWMGGIIPIFGTANYAQKFQGRVT
ITADKSTSTAYMELSSLRSEDTAVYYCAR
SEQ ID NO.: 79: candidate human model for 25D8 heavy chain variable
domain
QVQLVQSGAEVKKPGASVKVSCKASGYTFTSYYMHWVRQAPGQGLEWMGIINPSGGSTSYAQKFQGRVT
MTRDTSTSTVYMELSSLRSEDTAVYYCAR
SEQ ID NO.: 80: candidate human model for 25D8 heavy chain variable
domain
QVQLVQSGAEVKKPGASVKVSCKASGYTFTSYAISWVRQAPGQGLEWMGWINPGNGDTNYAQKFQGRVT
ITADTSTSTAYMELSSLRSEDTAVYYCARGGRGDYFDYWGQGTLVTVSS
SEQ ID NO.: 81: candidate human model for 25E9 light chain variable
domain
DIVMTQSPLSLPVTPGEPASISCRSSQSLLHSTGNNYLDWYLQKPGQSPQLLIYLGSNRASGVPDRFSG
SGSGTDFTLKISRVEAEDVGVYYCMQFLQTPLTFGGGTKVEIK
SEQ ID NO.: 82: candidate human model for 25E9 light chain variable
domain
DIVMTQTPLSLPVTPGEPASISCRASQSLLHSNGYNYLDWYLQKPGQSPQLLIYLGSNRASGVPDRFSG
SGSGTDFTLRISRVEAEDVGVYYCMQGLQTPLTFGGGTKVEIK
SEQ ID NO.: 83: candidate human model for 25E9 light chain variable
domain
DIVMTQSPLSLPVTPGEPASISCRSSQSLLHSNGYNYLDWYLQKPGQSPQLLIYLGSNRASGVPDRFSG
SGSGTDFTLKISRVEAEDVGVYYCMQALQ
SEQ ID NO.: 84: candidate human model for 25E9 light chain variable
domain
DIVMTQPPLSLPVTPGEPASISCRSSQSLLHSNGYNYLDWYMQKPGQSPQLLIYLGSNRASGVPDRFSG
SGSGTDFTLKISRVEAEDVGVYYCMQALQ
SEQ ID NO.: 85: candidate human model for 25E9 light chain variable
domain
DIVMTQSPLSLPVTPGEPASISCRSSQSLLHSDGNNYLNWYLQKPGQSPQLLIYLVSNRASGVPDRFSG
SGSGTDFTLKISRVEAEDVGVYYCMQALQPRXTFGQGTKVEIK
SEQ ID NO.: 86: candidate human model for 25E9 heavy chain variable
domain
QVQLQQSGAEVKKPGSSVKVSCKASGGTFSTYSISWVRQAPGHGLEWMGRIFPLLGVAKYAQKFQGRVT
ITADKSTSTAYMELSSLRSEDTAVYYCAVPRSSSYWFDPWGQGTLVTVSS 5
SEQ ID NO.: 87: candidate human model for 25E9 heavy chain variable
domain
EVQLVQSGAEVKKPGSSVKVSCKASGGTFSSYAISWVRQAPGQGLEWMGGIIPIFGTANYAQKFQGRVT
ITADESTSTAYMELSSLRSEDTAVYYCARGNYDSSGYDDAFDIWGQGTMVTVSS
SEQ ID NO.: 88: candidate human model for 25E9 heavy chain variable
domain
EVQLVQSGAEVKKPGSSVKLSCKASGDTFSSRPVSWVRQAPGQGLEWMGGIIPIFRTTNYAQKFQGRVT
ITADESMTTAYLELRGLTSDDTAVYYCATTRMKITVFASTFDYWGQGTLVTVSS
NO.: 89: candidate human model for 25E9 heavy chain variable
domain
QVQLVQSGAEVKKPGSSVKVSCKASGGTFSSYAISWVRQAPGQGLEWMGGIIPIFGTANYAQKFQGRVT
ITADKSTSTAYMELSSLRSEDTAVYYC
SEQ ID NO.: 90: candidate human model for 25E9 heavy chain variable
domain
QVQLVQSGAEVKKPGASVKVSCKASGYTFTSYYMHWVRQAPGQGLEWMGIINPSGGSTSYAQKFQGRVT
MTRDTSTSTVYMELSSLRSEDTAVYYC
SEQ ID NO.: 91: candidate human model for 25E9 heavy chain variable
domain
EVQLVQSGAEVKKPGATVKISCKVSGYTFTDYYMHWVQQAPGKGLEWMGLVDPEDGETIYAEKFQGRVT
ITADTSTDTAYMELSSLRSEDTAVYYCAT
SEQ ID NO.: 92: candidate human model for 25E9 heavy chain variable
domain
QVQLVQSGAEVKKPGASVKVSCKASGYTFTSYAISWVRQAPGQGLEWMGWINPGNGDTNYAQKFQGRVT
ITADTSTSTAYMELSSLRSEDTAVYYCARAPGYGSRGDYXFDYWGQGTLVTVSS
SEQ ID NO.: 93 (25B8 mouse light chain)
DIVMTQAAPSVPVTPGESVSISCRSTKSLLHSNGNTYLYWFLQRPGQSPQLLIYRMSNLASGVPDRFSG
SGSGTAFTLRISRVEAEDVGVYYCMQHLEYPFTFGGGTKLEIKVAAPSVFIFPPSDEQLKSGTASVVCL
LNNEYPREAKVQWKVDNALQSGNSQESVTEQDSKDSTYSLSSTLTLSKADYEKHKVYACEVTHQGLSSP
VTKSFNRGEC
SEQ ID NO.: 94 (25B8 mouse heavy chain)
EIQLQQSGVELVRPGASVTLSCKASGYTFTDYDMHWVKQTPVHGLEWIGTIDPETGGTAYNQKFKGKAT
LTADRSSTTAYMELSSLTSEDSAVYYCTTFYYSHYNYDVGFAYWGQGTLVTVSAASTKGPSVFPLAPCS
RSTSESTAALGCLVKDYFPEPVTVSWNSGALTSGVHTFPAVLQSSGLYSLSSVVTVPSSNFGTQTYTCN
VDHKPSNTKVDKTVERKCCVECPPCPAPPVAGPSVFLEPPKPKDTLMISRTPEVTCVVVDVSHEDPEVQ
FNWYVDGVEVHNAKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGSFFLYSKLTV
DKSRWQQGNVFSCSVMHEALHNHYTQKSLSLSPGK
SEQ ID NO.: 95 (25B8 light chain mouse variable domain)
DIVMTQAAPSVPVTPGESVSISCRSTKSLLHSNGNTYLYWFLQRPGQSPQLLIYRMSNLASGVPDRFSG
SGSGTAFTLRISRVEAEDVGVYYCMQHLEYPFTFGGGTKLEIK
SEQ ID NO.: 96 (2588 heavy chain mouse variable domain)
EIQLQQSGVELVRPGASVTLSCKASGYTFTDYDMHWVKQTPVHGLEWIGTIDPETGGTAYNQKFKGKAT
LTADRSSTTAYMELSSLTSEDSAVYYCTTFYYSHYNYDVGFAYWGQGTLVTVSA
SEQ ID NO.: 97 (25B8 CDRL1)
RSTKSLLHSNGNTYLY
SEQ ID NO.: 98 (25B8 CDRL2)
RMSNLAS
SEQ ID NO.: 99 (25B8 CDRL3)
MQHLEYPFT
SEQ ID NO.: 100 (25B8 CDRH1)
GYTFTDYDMH
SEQ ID NO.: 101 (25B8 CDRH2)
TIDPETGGTA
SEQ ID NO.: 102 (2588 CDRH3)
TTFYYSHYNYDVGFAY
SEQ ID NO.: 103 (#32A1 CDRH1)
DYFMN
SEQ ID NO. :104 (#32A1 CDRH2)
QIRNKIYTYATFYAESLEG
SEQ ID NO. :105 (#32A1 CDRH3)
SLTGGDYFDY
SEQ ID NO. :106 (#32A1 CDRL1)
RASQSVTISGYSFIH
SEQ ID NO. :107 (#32A1 CDRL2)
RASNLAS
SEQ ID NO.: 108 (#32A1 CDRL3)
QQSRKSPWT
SEQ ID NO.: 109 (#32A1 CDRH2 v2)
QIRNKIYTYATFYA
SEQ ID NO.: 110 : Rhesus monkey Siglec-15 cDNA:
atggaaagetccatccggctgctggcctgcttggcgtgtgtcctcccgacaggctcatttgtgagaact
aaaatagatactacggagaacttactcaacacagaggtgcacagctcgccagcgcagcgctggtccatg
caggtgccagccgaggtgagcgcggcggcaggcgacgcagcagtgctgccctgcaccttcactcacccg
caccgccactacgacgggccgctgactgccatctggcgcgcgggcgagccctacgcgggcccgcaggtg
ttccgctgcgctgcggcgcggggcagcgagctctgccagacggcgctgagcctgcacggccgcttccgg
ctgctgggcaacccgcgccgcaacgacctctcgctgcgcgtcgagcgcctcgccctggccgacgaccgc
cgctacttctgccgtgtcgagttcgccggcgacgtccacgaccgctacgagagccgccacggcgtccgg
ctgcacgtgaccgccgcgccgcggatcatcaacatctcggtgctgcccggccccgcgcacgccttccgt
gcgctctgcactgccgaaggggagccgccgcccgccctcgcctggtctggcccggccctgggcaatggc
tcggccgccgtgccgagctcgggtcagggtcacggccacctggtgaccgccgaactgcccgcactgaac
cacgacggccgctacacgtgtacggccgccaacagcctgggccgctccgaggccagcgtctacctgttc
cgcttccatggcgccagcggggcctcgacggtcgccctcctgctcggcgcgctcggcctcaaggcgctg
ctgctgctcggggtcctggccgctggcgtcgcccgccaccgcccagagcatctgaacaccccagacact
ccaccacggttccaggcccaggagtccaattatgaaaatttaagccagatgaatccccggagcccacca
gccgccatgtgctcaccgtga
SEQ ID NO.: 111: Rhesus monkey Siglec-15 amino acid
MESSIRLLACLACVLPTGSFVRTKIDTTENLLNTEVHSSPAQRWSMQVPAEVSAAAGDAAVLPCTFTHP
HRHYDGPLTAIWRAGEPYAGPQVFRCAAARGSELCQTALSLHGRFRLLGNPRRNDLSLRVERLALADDR
RYFCRVEFAGDVHDRYESRHGVRLHVTAAPRIINISVLPGPAHAFRALCTAEGEPPPALAWSGPALGNG
SAAVPSSGQGHGHLVTAELPALNHDGRYTCTAANSLGRSEASVYLFRFHGASGASTVALLLGALGLKAL
LLLGVLAAGVARHRPEHLNTPDTPPRFQAQESNYENLSQMNPRSPPAAMCSP

1. A method of treating osteogenesis imperfecta type VI, the method comprising administering an antibody or an antigen-binding fragment thereof capable of specific binding to Siglec-15 to an individual in need.

2. A method of treating a bone disease associated with an imbalance or dis-regulation of PEDF or in the PEDF pathway, the method comprising administering an antibody or an antigen-binding fragment thereof capable of specific binding to Siglec-15 to an individual in need.

3. The method of claim 1, wherein the bone disease is associated with an inactivation, loss of function, loss of expression, loss of secretion or mutation of PEDF.

4. The method of claim 1, wherein the bone disease is associated with an inactivation, loss of function, loss of expression, loss of secretion or mutation in a downstream effector of PEDF.

5. The method of claim 1, wherein the bone disease is associated with a loss of function, loss of expression, loss of secretion or mutation of a regulator that positively affects the expression or secretion of PEDF.

6. The method of claim 1, wherein the bone disease is associated with a gain of function, expression, secretion of a regulator that negatively affects the expression or secretion of PEDF.

7. The method of claim 2, wherein the individual in need suffers or is susceptible of suffering from osteogenesis imperfecta.

8. The method of claim 1, wherein the individual in need is an infant or a child.

9. The method of claim 1, wherein the individual in need has a mutation in the SERPINF1 gene.

10. The method of claim 1, wherein the antibody or antigen-binding fragment thereof is capable of inhibiting osteoclast differentiation, osteoclast formation or osteoclast activity.

11. The method of claim 1, wherein the antibody or antigen-binding fragment thereof is capable of inhibiting osteoclast differentiation, osteoclast formation or osteoclast activity in an in vitro or in vivo assay.

12. The method of claim 1, wherein the antibody or antigen-binding fragment thereof is capable of inhibiting bone resorption.

13. The method of claim 1, wherein the antibody or antigen-binding fragment thereof is a polyclonal antibody, a monoclonal antibody, a chimeric antibody, a human antibody, a humanized antibody, a hybrid antibody or an antigen-binding fragment thereof.

14.-20. (canceled)

21. An anti-Siglec-15 antibody for use in the treatment of ostoegenesis imperfecta type VI.


SEQUENCE LISTING (111 sequences)

SEQ ID NO: 1 - DNA, 987 bp, Homo sapiens
  Human Siglec-15 cDNA
atggaaaagtccatctggctgctggcctgcttggcgtgggttctcccgacaggctcatttgtgagaactaaaatagatactacggagaacttgctcaacacagaggtgcacagctcgccagcgcagcgctggtccatgcaggtgccacccgaggtgagcgcggaggcaggcgacgcggcagtgctgccctgcaccttcacgcacccgcaccgccactacgacgggccgctgacggccatctggcgcgcgggcgagccctatgcgggcccgcaggtgttccgctgcgctgcggcgcggggcagcgagctctgccagacggcgctgagcctgcacggccgcttccggctgctgggcaacccgcgccgcaacgacctctcgctgcgcgtcgagcgcctcgccctggctgacgaccgccgctacttctgccgcgtcgagttcgccggcgacgtccatgaccgctacgagagccgccacggcgtccggctgcacgtgacagccgcgccgcggatcgtcaacatctcggtgctgcccagtccggctcacgccttccgcgcgctctgcactgccgaaggggagccgccgcccgccctcgcctggtccggcccggccctgggcaacagcttggcagccgtgcggagcccgcgtgagggtcacggccacctagtgaccgccgaactgcccgcactgacccatgacggccgctacacgtgtacggccgccaacagcctgggccgctccgaggccagcgtctacctgttccgcttccatggcgccagcggggcctcgacggtcgccctcctgctcggcgctctcggcttcaaggcgctgctgctgctcggggtcctggccgcccgcgctgcccgccgccgcccagagcatctggacaccccggacaccccaccacggtcccaggcccaggagtccaattatgaaaatttgagccagatgaacccccggagcccaccagccaccatgtgctcaccgtga

SEQ ID NO: 2 - protein, 328 aa, Homo sapiens
  Human Siglec-15 Polypeptide (1-328)
MEKSIWLLACLAWVLPTGSFVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGASTVALLLGALGFKALLLLGVLAARAARRRPEHLDTPDTPPRSQAQESNYENLSQMNPRSPPATMCSP

SEQ ID NO: 3 - DNA, 1029 bp, Mus musculus
  Mouse Siglec-15 cDNA
atggaggggtccctccaactcctggcctgcttggcctgtgtgctccagatgggatcccttgtgaaaactagaagagacgcttcgggggatctgctcaacacagaggcgcacagtgccccggcgcagcgctggtccatgcaggtgcccgcggaggtgaacgcggaggctggcgacgcggcggtgctgccctgcaccttcacgcacccgcaccgccactacgacgggccgctgacggccatctggcgctcgggcgagccgtacgcgggcccgcaggtgttccgctgcaccgcggcgccgggcagcgagctgtgccagacggcgctgagcctgcacggccgcttccgcctgctgggcaacccgcgccgcaacgacctgtccctgcgcgtcgagcgcctcgccctggcggacagcggccgctacttctgccgcgtggagttcaccggcgacgcccacgatcgctatgagagtcgccatggggtccgtctgcgcgtgactgcagctgcgccgcggatcgtcaacatctcggtgctgccgggccccgcgcacgccttccgcgcgctctgcaccgccgagggggagcccccgcccgccctcgcctggtcgggtcccgccccaggcaacagctccgctgccctgcagggccagggtcacggctaccaggtgaccgccgagttgcccgcgctgacccgcgacggccgctacacgtgcacggcggccaatagcctgggccgcgccgaggccagcgtctacctgttccgcttccacggcgcccccggaacctcgaccctagcgctcctgctgggcgcgctgggcctcaaggccttgctgctgcttggcattctgggagcgcgtgccacccgacgccgactagatcacctggtcccccaggacacccctccacggtctcaggctcaggagtccaattatgaaaatttgagccagatgagtcctccaggccaccagctgccacgtgtttgctgtgaggaactcctcagccatcaccatctagtcattcaccatgagaaataa

SEQ ID NO: 4 - protein, 342 aa, Mus musculus
  Mouse Siglec-15 Polypeptide
MEGSLQLLACLACVLQMGSLVKTRRDASGDLLNTEAHSAPAQRWSMQVPAEVNAEAGDAAVLPCTFTHPHRHYDGPLTAIWRSGEPYAGPQVFRCTAAPGSELCQTALSLHGRFRLLGNPRRNDLSLRVERLALADSGRYFCRVEFTGDAHDRYESRHGVRLRVTAAAPRIVNISVLPGPAHAFRALCTAEGEPPPALAWSGPAPGNSSAALQGQGHGYQVTAELPALTRDGRYTCTAANSLGRAEASVYLFRFHGAPGTSTLALLLGALGLKALLLLGILGARATRRRLDHLVPQDTPPRSQAQESNYENLSQMSPPGHQLPRVCCEELLSHHHLVIHHEK

SEQ ID NO: 5 - protein, 239 aa, Artificial Sequence
  25E9 Light Chain Chimeric
MVLQTQVFISLLLWISGAYGDIVMTQAAPSVPVTPGESVSISCRSTKSLLHSNGNTYLYWFLQRPGQSPQLLIYRMSNLASGVPDRFSGSGSGTAFTLRISRVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 112 aa, Artificial Sequence
  25E9 Light Chain Mouse Variable Domain
DIVMTQAAPSVPVTPGESVSISCRSTKSLLHSNGNTYLYWFLQRPGQSPQLLIYRMSNLASGVPDRFSGSGSGTAFTLRISRVEAEDVGVYYCMQHLEYPFTFGGGTKLEIK

SEQ ID NO: 7 - protein, 239 aa, Artificial Sequence
  25E9 Light Chain Humanized Variant 1 (L1)
MVLQTQVFISLLLWISGAYGDIVMTQSPLSLPVTPGEPASISCRSTKSLLHSNGNTYLYWYLQKPGQSPQLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 112 aa, Artificial Sequence
  25E9 Light Chain Humanized Variant 1 (VL1) Variable Domain
DIVMTQSPLSLPVTPGEPASISCRSTKSLLHSNGNTYLYWYLQKPGQSPQLLIYRMSNLASGVPDRFSGSGSGTDFTLKISRVEAEDVGVYYCMQHLEYPFTFGGGTKVEIK

SEQ ID NO: 9 - protein, 239 aa, Artificial Sequence
  5E9 Light Chain Humanized Variant 2 (L2)
MVLQTQVFISLLLWISGAYGDIVMTQSPLSLPVTPGEPASISCRSTKSLLHSNGNTYLYWFLQKPGQSPQLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 112 aa, Artificial Sequence
  25E9 Light Chain Humanized Variant 2 Variable Domain (VL2)
DIVMTQSPLSLPVTPGEPASISCRSTKSLLHSNGNTYLYWFLQKPGQSPQLLIYRMSNLASGVPDRFSGSGSGTDFTLKISRVEAEDVGVYYCMQHLEYPFTFGGGTKVEIK

SEQ ID NO: 11 - protein, 472 aa, Artificial Sequence
  25E9 Heavy Chain Chimeric
MDWTWRILFLVAAATGTHAEIQLQQSGVELVRPGASVTLSCKASGYTFTDYDMHWVKQTPVHGLEWIGTIDPETGGTAYNQKFKGKATLTADRSSTTAYMELSSLTSEDSAVYYCTSFYYTYSNYDVGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 123 aa, Artificial Sequence
  25E9 Heavy Chain Mouse Variable Domain
EIQLQQSGVELVRPGASVTLSCKASGYTFTDYDMHWVKQTPVHGLEWIGTIDPETGGTAYNQKFKGKATLTADRSSTTAYMELSSLTSEDSAVYYCTSFYYTYSNYDVGFAYWGQGTLVTVSA

SEQ ID NO: 13 - protein, 472 aa, Artificial Sequence
  25E9 Heavy Chain Humanized Variant 1 (H1)
MDWTWRILFLVAAATGTHAEIQLQQSGAEVKKPGSSVKVSCKASGYTFTDYDMHWVRQAPGQGLEWMGTIDPETGGTAYNQKFKGRVTITADKSTSTAYMELSSLRSEDTAVYYCTSFYYTYSNYDVGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 123 aa, Artificial Sequence
  25E9 Heavy Chain Humanized Variant 1 Variable Domain (VH1)
EIQLQQSGAEVKKPGSSVKVSCKASGYTFTDYDMHWVRQAPGQGLEWMGTIDPETGGTAYNQKFKGRVTITADKSTSTAYMELSSLRSEDTAVYYCTSFYYTYSNYDVGFAYWGQGTLVTVSS

SEQ ID NO: 15 - protein, 472 aa, Artificial Sequence
  25E9 Heavy Chain Humanized Variant 2 (H2)
MDWTWRILFLVAAATGTHAEIQLQQSGAEVKKPGSSVKVSCKASGYTFTDYDMHWVRQAPGQGLEWIGTIDPETGGTAYNQKFKGRATLTADRSTSTAYMELSSLRSEDTAVYYCTSFYYTYSNYDVGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 123 aa, Artificial Sequence
  25E9 Heavy Chain Humanized Variant 2 Variable Domain (VH2)
EIQLQQSGAEVKKPGSSVKVSCKASGYTFTDYDMHWVRQAPGQGLEWIGTIDPETGGTAYNQKFKGRATLTADRSTSTAYMELSSLRSEDTAVYYCTSFYYTYSNYDVGFAYWGQGTLVTVSS

SEQ ID NO: 17 - protein, 472 aa, Artificial Sequence
  25E9 Heavy Chain Humanized Variant 3 (H3)
MDWTWRILFLVAAATGTHAEIQLQQSGAEVKKPGSSVKVSCKASGYTFTDYDMHWVKQAPGQGLEWIGTIDPETGGTAYNQKFKGKATLTADRSTSTAYMELSSLRSEDTAVYYCTSFYYTYSNYDVGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 123 aa, Artificial Sequence
  25E9 Heavy Chain Humanized Variant 3 Variable Domain (VH3)
EIQLQQSGAEVKKPGSSVKVSCKASGYTFTDYDMHWVKQAPGQGLEWIGTIDPETGGTAYNQKFKGKATLTADRSTSTAYMELSSLRSEDTAVYYCTSFYYTYSNYDVGFAYWGQGTLVTVSS

SEQ ID NO: 19 - protein, 472 aa, Artificial Sequence
  25E9 Heavy Chain Humanized Variant 4 (H4)
MDWTWRILFLVAAATGTHAEIQLQQSGAEVKKPGSSVKVSCKASGYTFTDYDMHWVKQAPGHGLEWIGTIDPETGGTAYNQKFKGKATLTADRSTSTAYMELSSLTSEDTAVYYCTSFYYTYSNYDVGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 123 aa, Artificial Sequence
  25E9 Heavy Chain Humanized Variant 4 Variable Domain (VH4)
EIQLQQSGAEVKKPGSSVKVSCKASGYTFTDYDMHWVKQAPGHGLEWIGTIDPETGGTAYNQKFKGKATLTADRSTSTAYMELSSLTSEDTAVYYCTSFYYTYSNYDVGFAYWGQGTLVTVSS

SEQ ID NO: 21 - protein, 239 aa, Artificial Sequence
  25D8 Light Chain Chimeric
MVLQTQVFISLLLWISGAYGDIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 112 aa, Artificial Sequence
  25D8 Light Chain Mouse Variable Domain
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIK

SEQ ID NO: 23 - protein, 239 aa, Artificial Sequence
  25D8 Light Chain Humanized
MVLQTQVFISLLLWISGAYGDIVMTQTPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 112 aa, Artificial Sequence
  25D8 Light Chain Humanized Variable Domain
DIVMTQTPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGGGTKVEIK

SEQ ID NO: 25 - protein, 463 aa, Artificial Sequence
  25D8 Heavy Chain Chimeric
MDWTWRILFLVAAATGTHAQVQVQQPGAELVKPGASVKLSCKASGYTFTSYWMHWVKQRPGQGLEWIGLINPSNARTNYNEKFNTKATLTVDKSSSTAYMQLSSLTSEDSAVYYCARGGDGDYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 118 aa, Artificial Sequence
  25D8 Heavy Chain Mouse Variable Domain
QVQVQQPGAELVKPGASVKLSCKASGYTFTSYWMHWVKQRPGQGLEWIGLINPSNARTNYNEKFNTKATLTVDKSSSTAYMQLSSLTSEDSAVYYCARGGDGDYFDYWGQGTTLTVSS

SEQ ID NO: 27 - protein, 463 aa, Artificial Sequence
  25D8 Heavy Chain Humanized
MDWTWRILFLVAAATGTHAQVQLQQSGAEVKKPGSSVKVSCKASGYTFTSYWMHWVRQAPGQGLEWMGLINPSNARTNYNEKFNTRVTITADKSTSTAYMELSSLRSEDTAVYYCARGGDGDYFDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 118 aa, Artificial Sequence
  25D8 Heavy Chain Humanized Variable Domain
QVQLQQSGAEVKKPGSSVKVSCKASGYTFTSYWMHWVRQAPGQGLEWMGLINPSNARTNYNEKFNTRVTITADKSTSTAYMELSSLRSEDTAVYYCARGGDGDYFDYWGQGTTVTVSS

SEQ ID NO: 29 - protein, 468 aa, Artificial Sequence
  25E9 Heavy Chain Humanized Variant 1
MDWTWRILFLVAAATGTHAEIQLQQSGAEVKKPGSSVKVSCKASGYTFTDYDMHWVRQAPGQGLEWMGTIDPETGGTAYNQKFKGRVTITADKSTSTAYMELSSLRSEDTAVYYCTSFYYTYSNYDVGFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 468 aa, Artificial Sequence
  25E9 Heavy Chain Chimeric
MDWTWRILFLVAAATGTHAEIQLQQSGVELVRPGASVTLSCKASGYTFTDYDMHWVKQTPVHGLEWIGTIDPETGGTAYNQKFKGKATLTADRSSTTAYMELSSLTSEDSAVYYCTSFYYTYSNYDVGFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 330 aa, Homo sapiens [sequence omitted]

SEQ ID NO: 32 - protein, 326 aa, Homo sapiens
  Human IgG2 constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 112 aa, Artificial Sequence
  25E9 Light Chain Variable Domain Generic (Consensus 1)
DIVMTQXXXSXPVTPGEXXSISCRSTKSLLHSNGNTYLYWXLQXPGQSPQLLIYRMSNLASGVPDRFSGSGSGTXFTLXISRVEAEDVGVYYCMQHLEYPFTFGGGTKXEIK

SEQ ID NO: 34 - protein, 112 aa, Artificial Sequence; identical to SEQ ID NO: 33
  25E9 Light Chain Variable Domain Generic (consensus 2)

SEQ ID NO: 35 - protein, 112 aa, Artificial Sequence; identical to SEQ ID NO: 33
  25E9 Light Chain Variable Domain Generic (consensus 3)

SEQ ID NO: 36 - protein, 123 aa, Artificial Sequence
  25E9 Heavy Chain Variable Domain Generic (consensus 1)
EIQLQQSGXEXXXPGXSVXXSCKASGYTFTDYDMHWVXQXPXXGLEWXGTIDPETGGTAYNQKFKGXXTXTADXSXXTAYMELSSLXSEDXAVYYCTSFYYTYSNYDVGFAYWGQGTLVTVSX

SEQ ID NO: 37 - protein, 123 aa, Artificial Sequence; identical to SEQ ID NO: 36
  25E9 Heavy Chain Variable Domain Generic (consensus 2)

SEQ ID NO: 38 - protein, 123 aa, Artificial Sequence; identical to SEQ ID NO: 36
  25E9 heavy chain variable domain generic (consensus 3)

SEQ ID NO: 39 - protein, 112 aa, Artificial Sequence
  25D8 light chain variable domain generic (consensus 1)
DIVMTQXXXSXPVTXGXXASISCRSSKSLLHSNGITYLYWYLQKPGQSPQLLIYQMSNLASGVPDRFSXSGSGTDFTLXISRVEAEDVGVYYCAQNLELPYTFGGGTKXEIK

SEQ ID NO: 40 - protein, 112 aa, Artificial Sequence; identical to SEQ ID NO: 39
  25D8 light chain variable domain generic (consensus 2)

SEQ ID NO: 41 - protein, 112 aa, Artificial Sequence; identical to SEQ ID NO: 39
  25D8 light chain variable domain generic (consensus 3)

SEQ ID NO: 42 - protein, 118 aa, Artificial Sequence
  25D8 heavy chain variable domain generic (consensus 1)
QVQXQQXGAEXXKPGXSVKXSCKASGYTFTSYWMHWVXQXPGQGLEWXGLINPSNARTNYNEKFNTXXTXTXDKSXSTAYMXLSSLXSEDXAVYYCARGGDGDYFDYWGQGTTXTVSS

SEQ ID NO: 43 - protein, 118 aa, Artificial Sequence; identical to SEQ ID NO: 42
  25D8 heavy chain variable domain generic (consensus 2)

SEQ ID NO: 44 - protein, 118 aa, Artificial Sequence; identical to SEQ ID NO: 42
  25D8 heavy chain variable domain generic (consensus 3)

SEQ ID NO: 45 - protein, 467 aa, Artificial Sequence
  25D8 Heavy Chain Chimeric
MDWTWRILFLVAAATGTHAQVQVQQPGAELVKPGASVKLSCKASGYTFTSYWMHWVKQRPGQGLEWIGLINPSNARTNYNEKFNTKATLTVDKSSSTAYMQLSSLTSEDSAVYYCARGGDGDYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 467 aa, Artificial Sequence
  Humanized 25D8 Heavy Chain Humanized
MDWTWRILFLVAAATGTHAQVQLQQSGAEVKKPGSSVKVSCKASGYTFTSYWMHWVRQAPGQGLEWMGLINPSNARTNYNEKFNTRVTITADKSTSTAYMELSSLRSEDTAVYYCARGGDGDYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 16 aa, Artificial Sequence
  25E9 CDRL1
RSTKSLLHSNGNTYLY

SEQ ID NO: 48 - protein, 7 aa, Artificial Sequence
  25E9 CDRL2
RMSNLAS

SEQ ID NO: 49 - protein, 9 aa, Artificial Sequence
  25E9 CDRL3
MQHLEYPFT

SEQ ID NO: 50 - protein, 10 aa, Artificial Sequence
  25E9 CDRH1
GYTFTDYDMH

SEQ ID NO: 51 - protein, 17 aa, Artificial Sequence
  25E9 CDRH2
TIDPETGGTAYNQKFKG

SEQ ID NO: 52 - protein, 14 aa, Artificial Sequence
  25E9 CDRH3
FYYTYSNYDVGFAY

SEQ ID NO: 53 - protein, 16 aa, Artificial Sequence
  25D8 CDRL1
RSSKSLLHSNGITYLY

SEQ ID NO: 54 - protein, 8 aa, Artificial Sequence
  25D8 CDRL2
QMSNLASG

SEQ ID NO: 55 - protein, 9 aa, Artificial Sequence
  25D8 CDRL3
AQNLELPYT

SEQ ID NO: 56 - protein, 10 aa, Artificial Sequence
  25D8 CDRH1
GYTFTSYWMH

SEQ ID NO: 57 - protein, 17 aa, Artificial Sequence
  25D8 CDRH2
LINPSNARTNYNEKFNT

SEQ ID NO: 58 - protein, 9 aa, Artificial Sequence
  25D8 CDRH3
GGDGDYFDY

SEQ ID NO: 59 - protein, 468 aa, Artificial Sequence
  25E9 Heavy Chain Humanized Variant 2 (H2)
MDWTWRILFLVAAATGTHAEIQLQQSGAEVKKPGSSVKVSCKASGYTFTDYDMHWVRQAPGQGLEWIGTIDPETGGTAYNQKFKGRATLTADRSTSTAYMELSSLRSEDTAVYYCTSFYYTYSNYDVGFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 60 - protein, 468 aa, Artificial Sequence
  25E9 Heavy Chain Humanized Variant 3 H3
MDWTWRILFLVAAATGTHAEIQLQQSGAEVKKPGSSVKVSCKASGYTFTDYDMHWVKQAPGQGLEWIGTIDPETGGTAYNQKFKGKATLTADRSTSTAYMELSSLRSEDTAVYYCTSFYYTYSNYDVGFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 61 - protein, 468 aa, Artificial Sequence
  25E9 Heavy Chain Humanized Variant 4 (H4)
MDWTWRILFLVAAATGTHAEIQLQQSGAEVKKPGSSVKVSCKASGYTFTDYDMHWVKQAPGHGLEWIGTIDPETGGTAYNQKFKGKATLTADRSTSTAYMELSSLTSEDTAVYYCTSFYYTYSNYDVGFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 62 - DNA, 336 bp, Artificial Sequence
  25E9 Light Chain mouse variable domain
gatattgtgatgacccaggctgcaccctctgtacctgtcactcctggagagtcagtatccatctcctgcaggtctactaagagtctcctgcatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatcctttcacgttcggaggggggaccaagctggaaataaaa

SEQ ID NO: 63 - DNA, 369 bp, Artificial Sequence
  25E9 Heavy Chain mouse variable domain
gagatccagctgcagcagtctggagttgagctggtgaggcctggggcttcagtgacgctgtcctgcaaggcttcgggctacacatttactgactatgacatgcactgggtgaagcagacacctgttcatggcctggaatggattggaactattgatcctgaaactggtggtactgcctacaatcagaagttcaagggcaaggccacactgactgcggacagatcctccaccacagcctacatggagctcagcagcctgacatctgaggactctgccgtctattactgtacaagtttctactatacttactctaattacgacgtggggtttgcttactggggccaagggactctggtcactgtctctgca

SEQ ID NO: 64 - DNA, 336 bp, Artificial Sequence
  Humanized 25E9 light chain variable domain-variant 1 (no signal sequence)
gacatcgtgatgacccagtcccccctgtccctgcctgtgacacctggcgagcccgcctccatctcctgccggtccaccaagtccctgctgcactccaacggcaacacctacctgtactggtatctgcagaagcccggccagtcccctcagctgctgatctaccggatgtccaacctggcctccggcgtgcccgacagattctccggctctggctccggcaccgacttcaccctgaagatctcccgggtggaagccgaggacgtgggcgtgtactactgcatgcagcacctggaataccccttcaccttcggcggaggcaccaaggtggaaatcaag

SEQ ID NO: 65 - DNA, 369 bp, Artificial Sequence
  humanized 25E9 heavy chain variable domain -variant 1 (no signal sequence)
gagattcagctgcagcagtcaggagccgaagtgaagaaacccggctccagcgtcaaggtgagttgcaaggcctccggatacactttcaccgactatgatatgcactgggtgagacaggcacctgggcagggtctggagtggatggggaccatcgatccagaaaccggcggaacagcctacaaccagaagtttaaaggtcgagtgacaattactgctgacaagtccaccagcacagcatatatggagctgtctagtctgcgttctgaagatacagccgtctactattgcacttctttctactacacctacagtaactacgacgtggggtttgcttactggggccagggaactctggtcaccgtgtcatcc

SEQ ID NO: 66 - protein, 112 aa, Artificial Sequence
  Candidate human model for 25D8 light chain variable domain
DIVMTQTPLSLPVTPGEPASISCRSSQSLLHSNGYNYLDWYLQKPGQSPQLLIYLGSNRASGVPDRFSGSGSGTDFTLKISRVEAEDVGVYYCMQGLQTPLTFGGGTKVEIK

SEQ ID NO: 67 - protein, 112 aa, Artificial Sequence
  Candidate human model for 25D8 light chain variable domain
DIVMTQTPLSLPVTPGEPASISCRASQSLLHSNGYNYLDWYLQKPGQSPQLLIYLGSNRASGVPDRFSGSGSGTDFTLRISRVEAEDVGVYYCMQGLQTPLTFGGGTKVEIK

SEQ ID NO: 68 - protein, 112 aa, Artificial Sequence
  Candidate human model for 25D8 light chain variable domain
DIVMTQSPLSLSVTPGQPASISCKSSQSLLHSDGKTYLYWYLQKPGQPPQLLIYEVSNRFSGVPDRFSGSGSGTDFTLKISRVEAEDVGVYYCMQSIQLPYTFGQGTKLEIK

SEQ ID NO: 69 - protein, 98 aa, Artificial Sequence
  Human model for 25D8 light chain variable domain
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQ

SEQ ID NO: 70 - protein, 98 aa, Artificial Sequence
  Candidate human model for 25D8 light chain variable domain
DIVMTQPPLSLPVTPGEPASISCRSSQSLLHSNGYNYLDWYLQKPGQSPQLLIYLGSNRASGVPDRFSGSGSGTDFTLKISRVEAEDVGVYYCMQALQ

SEQ ID NO: 71 - protein, 99 aa, Artificial Sequence
  Candidate human model for 25D8 light chain variable domain
DIVMTQTPLSLSVTPGQPASISCKSSQSLLHSDGKTYLYWYLQKPGQSPQLLIYEVSNRFSGVPDRFSGSGSGTDFTLKISRVEAEDVGVYYCMQSIQL

SEQ ID NO: 72 - protein, 112 aa, Artificial Sequence
  Candidate human model for 25D8 light chain variable domain
DIVMTQSPLSLPVTPGEPASISCRSSQSLLHSDGNNYLNWYLQKPGQSPQLLIYLVSNRASGVPDRFSGSGSGTDFTLKISRVEAEDVGVYYCMQALQPRXTFGQGTKVEIK

SEQ ID NO: 73 - protein, 119 aa, Artificial Sequence
  Candidate human model for 25D8 heavy chain variable domain
QVQLQQSGAEVKKPGSSVKVSCKASGGTFGSYAISWVRQAPGQGLEWMGRIIPILGIATYAQKFQGRVTITADKSTSTAYMDLSSLRSEDTAVYYCARGKGEFEGMDVWGQGTTVTVSS

SEQ ID NO: 74 - protein, 119 aa, Artificial Sequence
  Candidate human model for 25D8 heavy chain variable domain
QVQLQQSGAEVKKPGSSVKVSCKASGGTFSSYAISWVRQAPGQGLEWMGRIIPILGIANYAQKFQGRVTITADKSTSTAYMELSSLRSEDTAVYYCARDTHSWFAFDIWGQGTMVTVSS

SEQ ID NO: 75 - protein, 118 aa, Artificial Sequence
  Candidate human model for 25D8 heavy chain variable domain
EVQLVQSGAEMKKPGASVKVSCKASGYSFSIYNIHWVRQAPGQGLEWMGWIHAGTGNRKYSQVFQDRVTITRDTSASTSYMELSSLTSEDTAVYYCARDPNFGDFDSWGQGTLVTVSS

SEQ ID NO: 76 - protein, 118 aa, Artificial Sequence
  Candidate human model for 25D8 heavy chain variable domain
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARMYNWNFFDYWGQGTLVTVSS

SEQ ID NO: 77 - protein, 120 aa, Artificial Sequence
  Candidate human model for 25D8 heavy chain variable domain
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGDGYIQAFDYWGQGTLVTVSS

SEQ ID NO: 78 - protein, 98 aa, Artificial Sequence
  Candidate human model for 25D8 heavy chain variable domain
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAR

SEQ ID NO: 79 - protein, 98 aa, Artificial Sequence
  Candidate human model for 25D8 heavy chain variable domain
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 80 - protein, 118 aa, Artificial Sequence
  Candidate human model for 25D8 heavy chain variable domain
QVQLVQSGAEVKKPGASVKVSCKASGYTFTSYAISWVRQAPGQGLEWMGWINPGNGDTNYAQKFQGRVTITADTSTSTAYMELSSLRSEDTAVYYCARGGRGDYFDYWGQGTLVTVSS

SEQ ID NO: 81 - protein, 112 aa, Artificial Sequence
  Candidate human model for 25E9 light chain variable domain
DIVMTQSPLSLPVTPGEPASISCRSSQSLLHSTGNNYLDWYLQKPGQSPQLLIYLGSNRASGVPDRFSGSGSGTDFTLKISRVEAEDVGVYYCMQFLQTPLTFGGGTKVEIK

SEQ ID NO: 82 - protein, 112 aa, Artificial Sequence; identical to SEQ ID NO: 67
  Candidate human model for 25E9 light chain variable domain

SEQ ID NO: 83 - protein, 98 aa, Artificial Sequence; identical to SEQ ID NO: 69
  Candidate human model for 25E9 light chain variable domain

SEQ ID NO: 84 - protein, 98 aa, Artificial Sequence; identical to SEQ ID NO: 70
  Candidate human model for 25E9 light chain variable domain

SEQ ID NO: 85 - protein, 112 aa, Artificial Sequence; identical to SEQ ID NO: 72
  candidate human model for 25E9 light chain variable domain

SEQ ID NO: 86 - protein, 119 aa, Artificial Sequence
  Candidate human model for 25E9 heavy chain variable domain
QVQLQQSGAEVKKPGSSVKVSCKASGGTFSTYSISWVRQAPGHGLEWMGRIFPLLGVAKYAQKFQGRVTITADKSTSTAYMELSSLRSEDTAVYYCAVPRSSSYWFDPWGQGTLVTVSS

SEQ ID NO: 87 - protein, 123 aa, Artificial Sequence
  Candidate human model for 25E9 heavy chain variable domain
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGNYDSSGYDDAFDIWGQGTMVTVSS

SEQ ID NO: 88 - protein, 123 aa, Artificial Sequence
  Candidate human model for 25E9 heavy chain variable domain
EVQLVQSGAEVKKPGSSVKLSCKASGDTFSSRPVSWVRQAPGQGLEWMGGIIPIFRTTNYAQKFQGRVTITADESMTTAYLELRGLTSDDTAVYYCATTRMKITVFASTFDYWGQGTLVTVSS

SEQ ID NO: 89 - protein, 96 aa, Artificial Sequence
  Candidate human model for 25E9 heavy chain variable domain
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYC

SEQ ID NO: 90 - protein, 96 aa, Artificial Sequence
  Candidate human model for 25E9 heavy chain variable domain
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYC

SEQ ID NO: 91 - protein, 98 aa, Artificial Sequence
  Candidate human model for 25E9 heavy chain variable domain
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 92 - protein, 123 aa, Artificial Sequence
  Candidate human model for 25E9 heavy chain variable domain
QVQLVQSGAEVKKPGASVKVSCKASGYTFTSYAISWVRQAPGQGLEWMGWINPGNGDTNYAQKFQGRVTITADTSTSTAYMELSSLRSEDTAVYYCARAPGYGSRGDYXFDYWGQGTLVTVSS

SEQ ID NO: 93 - protein, 217 aa, Artificial Sequence
  25B8 mouse light chain
DIVMTQAAPSVPVTPGESVSISCRSTKSLLHSNGNTYLYWFLQRPGQSPQLLIYRMSNLASGVPDRFSGSGSGTAFTLRISRVEAEDVGVYYCMQHLEYPFTFGGGTKLEIKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 449 aa, Artificial Sequence
  25B8 mouse heavy chain
EIQLQQSGVELVRPGASVTLSCKASGYTFTDYDMHWVKQTPVHGLEWIGTIDPETGGTAYNQKFKGKATLTADRSSTTAYMELSSLTSEDSAVYYCTTFYYSHYNYDVGFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 95 - protein, 112 aa, Artificial Sequence; identical to SEQ ID NO: 6
  25B8 light chain mouse variable domain

SEQ ID NO: 96 - protein, 123 aa, Artificial Sequence
  25B8 heavy chain mouse variable domain
EIQLQQSGVELVRPGASVTLSCKASGYTFTDYDMHWVKQTPVHGLEWIGTIDPETGGTAYNQKFKGKATLTADRSSTTAYMELSSLTSEDSAVYYCTTFYYSHYNYDVGFAYWGQGTLVTVSA

SEQ ID NO: 97 - protein, 16 aa, Artificial Sequence
  25B8 CDRL1
RSTKSLLHSNGNTYLY

SEQ ID NO: 98 - protein, 7 aa, Artificial Sequence
  25B8 CDRL2
RMSNLAS

SEQ ID NO: 99 - protein, 9 aa, Artificial Sequence
  25B8 CDRL3
MQHLEYPFT

SEQ ID NO: 100 - protein, 10 aa, Artificial Sequence
  25B8 CDRH1
GYTFTDYDMH

SEQ ID NO: 101 - protein, 10 aa, Artificial Sequence
  25B8 CDRH2
TIDPETGGTA

SEQ ID NO: 102 - protein, 16 aa, Artificial Sequence
  25B8 CDRH3
TTFYYSHYNYDVGFAY

SEQ ID NO: 103 - protein, 5 aa, Artificial Sequence
  #32A1 CDRH1
DYFMN

SEQ ID NO: 104 - protein, 19 aa, Artificial Sequence
  #32A1 CDRH2
QIRNKIYTYATFYAESLEG

SEQ ID NO: 105 - protein, 10 aa, Artificial Sequence
  #32A1 CDRH3
SLTGGDYFDY

SEQ ID NO: 106 - protein, 15 aa, Artificial Sequence
  #32A1 CDRL1
RASQSVTISGYSFIH

SEQ ID NO: 107 - protein, 7 aa, Artificial Sequence
  #32A1 CDRL2
RASNLAS

SEQ ID NO: 108 - protein, 9 aa, Artificial Sequence
  #32A1 CDRL3
QQSRKSPWT

SEQ ID NO: 109 - protein, 14 aa, Artificial Sequence
  #32A1 CDRH2 v2
QIRNKIYTYATFYA

SEQ ID NO: 110 - DNA, 987 bp, Macaca mulatta
  Rhesus monkey Siglec-15 cDNA
atggaaagctccatccggctgctggcctgcttggcgtgtgtcctcccgacaggctcatttgtgagaactaaaatagatactacggagaacttactcaacacagaggtgcacagctcgccagcgcagcgctggtccatgcaggtgccagccgaggtgagcgcggcggcaggcgacgcagcagtgctgccctgcaccttcactcacccgcaccgccactacgacgggccgctgactgccatctggcgcgcgggcgagccctacgcgggcccgcaggtgttccgctgcgctgcggcgcggggcagcgagctctgccagacggcgctgagcctgcacggccgcttccggctgctgggcaacccgcgccgcaacgacctctcgctgcgcgtcgagcgcctcgccctggccgacgaccgccgctacttctgccgtgtcgagttcgccggcgacgtccacgaccgctacgagagccgccacggcgtccggctgcacgtgaccgccgcgccgcggatcatcaacatctcggtgctgcccggccccgcgcacgccttccgtgcgctctgcactgccgaaggggagccgccgcccgccctcgcctggtctggcccggccctgggcaatggctcggccgccgtgccgagctcgggtcagggtcacggccacctggtgaccgccgaactgcccgcactgaaccacgacggccgctacacgtgtacggccgccaacagcctgggccgctccgaggccagcgtctacctgttccgcttccatggcgccagcggggcctcgacggtcgccctcctgctcggcgcgctcggcctcaaggcgctgctgctgctcggggtcctggccgctggcgtcgcccgccaccgcccagagcatctgaacaccccagacactccaccacggttccaggcccaggagtccaattatgaaaatttaagccagatgaatccccggagcccaccagccgccatgtgctcaccgtga

SEQ ID NO: 111 - protein, 328 aa, Macaca mulatta
  Rhesus monkey Siglec-15 amino acid
MESSIRLLACLACVLPTGSFVRTKIDTTENLLNTEVHSSPAQRWSMQVPAEVSAAAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIINISVLPGPAHAFRALCTAEGEPPPALAWSGPALGNGSAAVPSSGQGHGHLVTAELPALNHDGRYTCTAANSLGRSEASVYLFRFHGASGASTVALLLGALGLKALLLLGVLAAGVARHRPEHLNTPDTPPRFQAQESNYENLSQMNPRSPPAAMCSP